(19) United States
(12) Patent Application Publication
(10) Pub. No.: US 2025/0171482 A1
(43) Pub. Date: May 29, 2025

(54) DEACYLATION OF CARBOHYDRATE COMPOUNDS

(71) Applicants: ELYSIUM HEALTH INC., New York, NY (US); UNIVERSITY OF SOUTH ALABAMA, Mobile, AL (US)

(72) Inventors: Ryan DELLINGER, New York, NY (US); Marie MIGAUD, Mobile, AL (US); Jyothi DHUGURU, Mobile, AL (US)

(21) Appl. No.: 18/844,106

(22) PCT Filed: Mar. 10, 2023
(86) PCT No.: PCT/US2023/064099

§ 371 Date: Sep. 5, 2024

Related U.S. Application Data
(60) Provisional application No. 63/319,307, filed on Mar. 12, 2022.

(51) Int. Cl. C07H 1/06; C07H 3/02

(52) U.S. Cl. CPC C07H 1/06; C07H 3/02

(57) ABSTRACT

Compositions of de-acylated carbohydrate compounds and methods of making them are provided. The methods can be designed to produce the desired products under conditions of moderate temperature, moderate pH, and without the use of harsh solvents.

### BACKGROUND

Nicotinamide riboside (NR) and its reduced form, dihydronicotinamide riboside (NRH), are bioactive molecules under investigation for various biomedical and pharmaceutical uses. However, both materials suffer from some synthetic and stability limitations. In terms of production, existing NR and NRH syntheses use large molar equivalency of protic solvents for which either acidic or basic conditions are applied to assist in the removal of acetate groups present on the nucleoside following glycosylation of the tetraacetate riboside or the chlorotriacetate riboside by nicotinamide (NAM). These conditions complicate the synthesis of NR and NRH, making the production of these compounds costly, generating hazardous byproducts, and requiring technically constraining protocols and safety measures.

The synthesis of several other compounds with acyl-modified carbohydrate moiety suffers from similar problems.

Consequently, there is a need in the art for efficient means to produce compounds with carbohydrate scaffolds under milder and more atom-efficient reaction conditions.

### SUMMARY

The present disclosure describes a novel reaction that addresses the problems described above by producing compounds with carbohydrate groups through de-acylation in the presence of methanol and a basic amino acid. The reaction appears to generate little or no hazardous byproducts and can be performed under mild conditions of pH and temperature. The deacylation is useful in deesterification and deamidation, among other applications.

A method of deacylating an acylated carbohydrate is provided, the method comprising: (a) forming a reaction mixture by mixing the acylated carbohydrate with a basic amino acid and methanol; and (b) allowing the methanol to react with an acyl group of the acylated carbohydrate.

A method of deacylating a heteroaromatic-aryl amide is provided, the method comprising: (a) forming a reaction mixture by mixing the heteroaromatic-aryl amide with a basic amino acid and methanol; and (b) allowing the methanol to react with an amide group of the heteroaromatic-aryl amide.

A method of producing a deacylated carbohydrate from a corresponding acylated carbohydrate is provided, the method comprising: (a) forming a reaction mixture by mixing the acylated carbohydrate with a basic amino acid and methanol; and (b) allowing the methanol to react with the acylated carbohydrate under conditions sufficient to form the deacylated carbohydrate.

A method of producing a deacylated heteroaromatic amine from a corresponding heteroaromatic amide is provided, the method comprising: (a) forming a reaction mixture by mixing the heteroaromatic amide with a basic amino acid and methanol; and (b) allowing the methanol to react with the heteroaromatic amide under conditions sufficient to form the deacylated heteroaromatic amine.

A method of producing a composition of a basic amino acid and a deacylated carbohydrate is provided, the method comprising: (a) forming a reaction mixture by mixing an acylated carbohydrate with a basic amino acid and methanol; (b) allowing the methanol to react with the acylated carbohydrate under conditions sufficient to form the deacylated carbohydrate and a methyl ester; and (c) removing unreacted methanol from the reaction mixture.

A method of producing a composition of a basic amino acid and a deacylated heteroaromatic amine is provided, the method comprising: (a) forming a reaction mixture by mixing a heteroaromatic amide with a basic amino acid and methanol; (b) allowing the methanol to react with the heteroaromatic amide under conditions sufficient to form the deacylated heteroaromatic amine and a methyl ester; and (c) removing unreacted methanol from the reaction mixture.

A reaction mixture is provided, comprising a basic amino acid, an acylated carbohydrate, and methanol.

A reaction mixture is provided, comprising a basic amino acid, a heteroaromatic amide, and methanol.

A composition comprising a basic amino acid and a deacylated carbohydrate is provided that is the product of any one of the processes above.

A composition comprising a basic amino acid and a deacylated heteroaromatic amine is provided that is the product of any one of the processes above.

In any of the foregoing embodiments, the reaction mixture further comprises water. In some embodiments, the reaction mixture comprises methanol and water at a ratio of about 1:10 to about 1:1.

The above presents a simplified summary in order to provide a basic understanding of some aspects of the claimed subject matter. This summary is not an extensive overview. It is not intended to identify key or critical elements or to delineate the scope of the claimed subject matter. Its sole purpose is to present some concepts in a simplified form as a prelude to the more detailed description that is presented later.

### BRIEF DESCRIPTION OF THE DRAWINGS

The present disclosure can be better understood, by way of example only, with reference to the following drawings. The elements of the drawings are not necessarily to scale relative to each other, emphasis instead being placed upon clearly illustrating the principles of the disclosure.

FIG. 1 shows a graphical representation of nuclear magnetic resonance (NMR) spectra of nicotinamide riboside triacetate (NRTA) de-acetylation by several amino acids. Test reactions are shown, from top to bottom, for glycine, glutamic acid, histidine, and arginine.

FIG. 2 shows a schematic representation of a reaction for de-acetylation of NRTA using lysine.

FIG. 3A shows a schematic representation of a reaction for de-acetylation of NRTA using 1 equivalent of lysine.

FIG. 3B shows a schematic representation of a reaction for de-acetylation of NRHTA using 3 equivalents of lysine.

FIG. 4A shows a schematic representation of a reaction for deprotection of cytidine using lysine.

FIG. 4B shows a schematic representation of a reaction for deprotection of adenosine using lysine.

FIG. 4C shows a schematic representation of a reaction for deprotection of guanosine using lysine.

FIG. 5 shows a graphical representation of NMR spectra for cytidine.

FIG. 6 shows a graphical representation of NMR spectra for cytidine.

FIG. 7 shows a graphical representation of NMR spectra for adenosine.

FIG. 8 shows a graphical representation of NMR spectra for adenosine.

FIG. 9 shows a graphical representation of NMR spectra for triacetyl-guanosine.

FIG. 10 shows a graphical representation of NMR spectra for guanosine.

FIG. 11 shows a graphical representation of NMR spectra for guanosine.

FIG. 12 shows a graphical representation of NMR spectra for guanosine.

FIG. 13A shows a schematic representation of a reaction for deprotection of thymidine using lysine.

FIG. 13B shows a schematic representation of a reaction for deprotection of uridine using lysine.

FIG. 14 shows a graphical representation of NMR spectra for thymidine and lysine.

FIG. 15 shows a graphical representation of NMR spectra for uridine and lysine.

FIG. 16 shows a graphical representation of NMR spectra for uridine and lysine, with methyl ester peaks indicated prior to overnight drying.

FIG. 17A shows a schematic representation of a reaction for deprotection of guanosine using lysine.

FIG. 17B shows a schematic representation of a reaction for deprotection of adenosine using lysine.

FIG. 17C shows a schematic representation of a reaction for deprotection of guanosine using lysine.

FIG. 17D shows a schematic representation of a reaction for deprotection of cytidine using lysine.

FIG. 18 shows a graphical representation of NMR spectra for tetraacetyl-guanosine.

FIG. 19 shows a graphical representation of NMR spectra for guanosine.

FIG. 20 shows a graphical representation of NMR spectra for guanosine.

FIG. 21 shows a graphical representation of NMR spectra for 2′,3′-isopropylidene adenosine.

FIG. 22 shows a graphical representation of NMR spectra for 2′,3′-isopropylidene adenosine.

FIG. 23 shows a graphical representation of NMR spectra for lysine isolated from the de-acetylation of an adenosine-acetonide derivative.

FIG. 24 shows a graphical representation of NMR spectra for cytidine.

FIG. 25 shows a graphical representation of NMR spectra for NRTA de-acetylation using 1:1 NRTA:Lysine.

FIG. 26 shows a graphical representation of NMR spectra for NRTA de-acetylation using 1:3 NRTA:Lysine hydrate.

FIG. 27 shows a graphical representation of NMR spectra for NRTA de-acetylation using 1:3 NRTA:Lysine hydrate.

FIG. 28 shows a graphical representation of NMR spectra for NR product formation over time. Top represents NR, middle represents reaction after 4.5 h, and bottom represents reaction overnight. Reaction used 1:1 NRTA:Lysine.

FIG. 29 shows a graphical representation of NMR spectra for NR product formation. Top represents NR, middle represents NRT-Cl substrate, and bottom represents the product after recrystallization. Reaction used 1:1 NRTA:Lysine.

FIG. 30 shows a graphical representation of NMR spectra for NR and lysine after recrystallization.

FIG. 31 shows a graphical representation of NMR spectra for NRHTA de-acetylation. Top represents NRHTA with 3 equivalents of lysine, middle represents NRHTA with 2 equivalents of lysine, and bottom represents NRHTA with 1 equivalent of lysine.

FIG. 32 shows a graphical representation of NMR spectra for NRHTA de-acetylation with 1:3 NRHTA:Lysine in methanol. Top to bottom represents reaction times of 72 h, 48 h, 24 h, and overnight.

FIG. 33 shows a graphical representation of NMR spectra for NRHTA de-acetylation with 1:3 NRHTA:Lysine in methanol for an overnight reaction.

FIG. 34 shows a graphical representation of NMR spectra for NRHTA de-acetylation with 1:3 NRHTA:Lysine in methanol. Top represents NRH, middle represents the reaction after overnight, and the bottom represents the supernatant (methanol:ether).

FIG. 35 shows a graphical representation of NMR spectra for NRHTA de-acetylation with 1:3 NRHTA:Lysine in methanol. NRH is shown.

FIG. 36 shows a graphical representation of NMR spectra for NRHTA de-acetylation. NRH and lysine is shown.

FIG. 37 shows a graphical representation of NMR spectra for NRHTA de-acetylation. NRH and lysine is shown.

FIG. 38 shows a graphical representation of NMR spectra for NRHTA de-acetylation. Top represents NRH, middle represents NRH and lysine in dried solid from the reaction mixture, and the bottom represents NRH and lysine in dried supernatant from the reaction mixture.

FIG. 39 shows a graphical representation of NMR spectra for deprotection of uridine using lysine and methanol. Top to bottom represents 1:3 triacetyl uridine:lysine, 1:3 triacetyl uridine:lysine with 30 minutes ball milling, 1:1 triacetyl uridine:lysine after 24 h, and triacetyl uridine.

FIG. 40 shows a graphical representation of NMR spectra for uridine and lysine.

FIG. 41 shows a graphical representation of NMR spectra for cytidine and lysine.

FIG. 42 shows a graphical representation of NMR spectra for cytidine after recrystallization.

FIG. 43 shows a graphical representation of NMR spectra for adenosine after recrystallization.

FIG. 44 shows a graphical representation of NMR spectra for deprotection of uridine using lysine and methanol. Top represents reaction after overnight vacuum with no 2 ppm peak, while bottom represents reaction before complete drying with a 2 ppm peak.

FIG. 45 shows a graphical representation of NMR spectra for deprotection of NR triacetate to NR.

FIG. 46 shows a graphical representation of NMR spectra for deprotection of NR triacetate to NR. Conditions are 1:1 NRTA-Cl:lysine with methanol (500 μL) at room temperature for 5 h. Lysine catalyzes the removal of all acetates and hydrolysis of NR to NAM occurs approximately 40%.

FIG. 47 shows a graphical representation of NMR spectra for deprotection of NR triacetate to NR. Conditions are 1:1 NRTA-Cl:glycine with methanol (15-20 equivalents) at room temperature after 24 h.

FIG. 48 shows a graphical representation of NMR spectra for deprotection of NR triacetate to NR. Conditions are 1:1 NRTA-Cl:glutamic acid with methanol (15-20 equivalents) at room temperature after 24 h.

FIG. 49 shows a graphical representation of NMR spectra for deprotection of NR triacetate to NR. Conditions are 1:1 NRTA-Cl:histidine with methanol (15-20 equivalents) at room temperature after 24 h.

FIG. 50 shows a graphical representation of NMR spectra for deprotection of NR triacetate to NR. Conditions are 1:1 NRTA-Cl:arginine with methanol (15-20 equivalents) at room temperature after 24 h.

FIG. 51 shows a graphical representation of NMR spectra for deprotection of NR triacetate to NR. From top to bottom, the amino acid used with 15 equivalents of methanol is glycine, glutamic acid, histidine, and arginine.

FIG. 52 shows a graphical representation of NMR spectra for deprotection of triacetyl uridine to uridine. Conditions are 1:3 triacetyl-uridine:lysine with methanol (15-20 equivalents) at room temperature after 24 h. Conversion occurs with lysine. Partial conversion with no decomposition is observed.

FIG. 53 shows a graphical representation of NMR spectra for NRTA de-acetylation. Conditions are 1:1 NRTA:lysine with methanol (3-4 equivalents) with 30 min ball milling time.

FIG. 54 shows a graphical representation of NMR spectra for NRTA de-acetylation. Lysine catalyzes the removal of all acetates in circa 60%; hydrolysis of NR to NAM occurs (approximately 20%) with approximately 20% of monoacetylated mixture still present.

FIG. 55 shows a graphical representation of NMR spectra for deprotection of acetyl groups from NRHTA using lysine in methanol. Oxidation of NRH to NR and hydrolysis to NAM occurs over time, shown at (top to bottom) 96 h, 72 h, 48 h, 24 h, and 0 h.

FIG. 56 shows a schematic representation of NR formation using various amino acids.

FIG. 57 shows a reaction scheme of deacylation of NRTA to form NR with 3 equivalents of lysine in a 1:1 mixture of methanol (MeOH) and water at room temperature.

FIG. 58A shows exemplary NMR spectra of the reaction products of the reaction shown in FIG. 57. FIG. 58B shows an enlarged portion of the spectra in FIG. 58A, showing the NR and NAM peaks.

FIG. 59 shows a reaction scheme of deacylation of NRTA to form NR with 1 equivalent of lysine in a 1:1 mixture of methanol (MeOH) and water at room temperature.

FIG. 60 shows exemplary NMR spectra of the reaction of FIG. 59 after 3 hours, 5 hours, and 48 hours.

FIG. 61 shows a reaction scheme of deacylation of NRTA to form NR with 1 equivalent of lysine in a 1:1 mixture of methanol (MeOH) and water at 4° C.

FIG. 62A shows exemplary NMR spectra of the reaction of FIG. 61 after 3 hours, 5 hours, and 48 hours. FIG. 62B shows an enlarged portion of the 48 hours reaction spectra in FIG. 62A, showing the NR and NAM peaks.

FIG. 63 shows a reaction scheme of deacylation of NRTA to form NR with 3 equivalents of lysine in a 1:1 mixture of methanol (MeOH) and water at 4° C.

FIG. 64A shows exemplary NMR spectra of the reaction of FIG. 63 after 3 hours, 5 hours, and 48 hours. FIG. 64B shows an enlarged portion of the 48 hours reaction spectra in FIG. 64A, showing the NR and NAM peaks.

FIG. 65 shows a reaction scheme of deacylation of NRTA to form NR with 1 equivalent of lysine in a 1:1 mixture of methanol (MeOH) and water at −20° C.

FIG. 66A shows exemplary NMR spectra of the reaction of FIG. 65 after 3 hours, 5 hours, 48 hours, and 72 hours. FIG. 66B shows an enlarged portion of the 48 hours reaction spectra in FIG. 66A, showing the NR and NAM peaks.

FIG. 67 shows a reaction scheme of deacylation of NRTA to form NR with 3 equivalents of lysine in a 1:1 mixture of methanol (MeOH) and water at −20° C.

FIG. 68A shows exemplary NMR spectra of the reaction of FIG. 65 after 3 hours, 5 hours, 48 hours, and 72 hours. FIG. 68B shows an enlarged portion of the 48 hours reaction spectra in FIG. 68A, showing the NR peaks.

FIG. 69 shows a reaction scheme of the deacylation of 2′,3′, 5′-triacetyl adenosine to form adenosine in a 1:1 mixture of water:MeOH at 50° C.

FIG. 70A shows exemplary NMR spectra of the reaction of FIG. 69 after 3.5 hours. FIG. 70B shows exemplary NMR spectra of the reaction of FIG. 69 after 1 hour, 2.5 hours, and 3.5 hours.

FIG. 71 shows a reaction scheme of the deacylation of 5′acetyl-2′,3′-isopropylidene adenosine to form 2′,3′-isopropylidene adenosine in a 1:1 mixture of water:MeOH at 50° C.

FIG. 72 shows exemplary NMR spectra of the reaction of FIG. 71 after 3.5 hours.

FIG. 73 shows a reaction scheme of the deacylation of 3′,5′-diacetyl thymidine to form thymidine in a 1:1 mixture of water:MeOH at 50° C.

FIG. 74 shows exemplary NMR spectra of the reaction of FIG. 73 after 5 hours.

FIG. 75 shows a reaction scheme of the deacylation of N-acetyl-cytidine to form cytidine in a 1:1 mixture of water:MeOH at 50° C.

FIG. 76A shows exemplary NMR spectra of the reaction of FIG. 75, except in MeOH.

FIG. 76B shows exemplary NMR spectra of the reaction of FIG. 75. FIG. 76C shows exemplary NMR spectra of the reaction of FIG. 75, except in water.

FIG. 77 shows a reaction scheme of the deacylation of N-acetyl-2′,3′,5′-triacetyl guanosine to form guanosine in a 1:1 mixture of water:MeOH at 50° C.

FIG. 78A shows exemplary NMR spectra of the reaction of FIG. 77 after 3 hours. FIG. 78B shows exemplary NMR spectra of the reaction of FIG. 77 after 1 hour, 2.5 hours, and 3.5 hours

FIG. 79 shows a reaction scheme of the deacylation of 2′,3′,5′-triacetyl guanosine to form guanosine in a 1:1 mixture of water:MeOH at 50° C.

FIG. 80 shows exemplary NMR spectra of the reaction of FIG. 79 after 3.5 hours.

FIG. 81 shows a reaction scheme of the deacylation of 2′,3′,5′-triacetyl uridine to form uridine in a 1:1 mixture of water:MeOH at 50° C.

FIG. 82 shows exemplary NMR spectra of the reaction of FIG. 81 after 4 hours.

FIG. 83 shows a reaction scheme of the deacylation of N-benzoyl-cytidine to form cytidine in a 1:1 mixture of water:MeOH at 50° C.

FIG. 84 shows exemplary NMR spectra of the reaction of FIG. 83 after 24 hours.

FIG. 85A shows a summary of the deacylation reaction time of various ribosides in MeOH. FIG. 85B shows a summary of the deacylation reaction time of various ribosides in a 1:1 water:MeOH mixture.

FIG. 86 shows a reaction scheme of the deacylation of N-acetyl cytidine to form cytidine in a 1:1 mixture of water:MeOH with microwave irradiation.

FIG. 87A shows exemplary NMR spectra of the reaction of FIG. 86 after 210 seconds.

FIG. 87B shows exemplary NMR spectra of the reaction of FIG. 86 after 60 seconds, 90 seconds, 150 seconds, and 210 seconds.

FIG. 88 shows exemplary NMR spectra of the reaction of FIG. 86, the same reaction conducted in water alone, and the same reaction conducted in MeOH alone.

FIG. 89 shows exemplary NMR spectra of a deacylation reaction of NRTA to NR in a 3:1 mixture of water:MeOH with 1 equivalent of lysine at 20-25° C.

FIG. 90A shows exemplary NMR spectra of a deacylation reaction of NRTA to NR in a 3:1 mixture of water:MeOH with 1 equivalent of lysine at 4° C., after 24 hours. FIG. 90B shows exemplary NMR spectra of the same reaction as FIG. 90A, after 48 hours.

FIG. 91A shows exemplary NMR spectra of a deacylation reaction of NRTA to NR in a 3:1 mixture of water:MeOH with 3 equivalents of lysine at −20° C., after 24 hours. FIG. 91B shows exemplary NMR spectra of the same reaction as FIG. 91A, after 48 hours. FIG. 91C shows exemplary NMR spectra of the same reaction as FIG. 91A, after 72 hours.

FIG. 92 shows exemplary NMR spectra of a deacylation reaction of NRTA to NR with a molar amount of MeOH used as solvent and reagent and an excess of water (approximate ratio of water:MeOH is 24:1) with 1 equivalent of lysine at 20-25° C.

FIG. 93A shows exemplary NMR spectra of a deacylation reaction of NRTA to NR with a molar amount of MeOH used as solvent and reagent and an excess of water with 1 equivalent of lysine at 4° C., after 24 hours. FIG. 93B shows exemplary NMR spectra of the same reaction as FIG. 93A, after 48 hours.

FIG. 94A shows exemplary NMR spectra of a deacylation reaction of NRTA to NR with a molar amount of MeOH used as solvent and reagent and an excess of water with 3 equivalents of lysine at 4° C., after 24 hours. FIG. 94B shows exemplary NMR spectra of the same reaction as FIG. 94A, after 48 hours.

FIG. 95 shows a reaction scheme of the deacylation of N-acetyl-cytidine to form cytidine in MeOH with immobilized morpholine at 80° C.

FIG. 96 shows exemplary NMR spectra of the reaction of FIG. 95, before and after filtration (top and bottom panels, respectively).

FIG. 97 shows exemplary NMR spectra of the reaction of FIG. 95 using recycled immobilized morpholine, before and after filtration (top and bottom panels, respectively).

FIG. 98 shows exemplary NMR spectra comparing the results using a first batch of immobilized morpholine (top panel) vs. a second, recycled batch of immobilized morpholine (bottom panel).

### DETAILED DESCRIPTION

### A. Definitions

Unless otherwise defined, all terms (including technical and scientific terms) used herein have the same meaning as commonly understood by one of ordinary skill in the art of this disclosure. It will be further understood that terms, such as those defined in commonly used dictionaries, should be interpreted as having a meaning that is consistent with their meaning in the context of the specification and should not be interpreted in an idealized or overly formal sense unless expressly so defined herein. Well known functions or constructions may not be described in detail for brevity or clarity.

The terms “about” and “approximately” shall generally mean an acceptable degree of error or variation for the quantity measured given the nature or precision of the measurements. Typical, exemplary degrees of error or variation are within 20 percent (%), preferably within 10%, more preferably within 5%, and still more preferably within 1l % of a given value or range of values. Numerical quantities given in this description are approximate unless stated otherwise, meaning that the term “about” or “approximately” can be inferred when not expressly stated.

The terminology used herein is for the purpose of describing particular embodiments only and is not intended to be limiting. As used herein, the singular forms “a”, “an” and “the” are intended to include the plural forms as well (i.e., “at least one” of what is described), unless the context clearly indicates otherwise. In every case, use of singular articles and pronouns should be interpreted to support claims to at least one of what is described, and to support claims to exactly one of what is described.

The terms “first”, “second”, and the like are used herein to describe various features or elements, but these features or elements should not be limited by these terms. These terms are only used to distinguish one feature or element from another feature or element. Thus, a first feature or element discussed below could be termed a second feature or element, and similarly, a second feature or element discussed below could be termed a first feature or element without departing from the teachings of the present disclosure.

Terms such as “at least one of A and B” should be understood to mean “only A, only B, or both A and B.” The same construction should be applied to longer list (e.g., “at least one of A, B, and C”).

Terms such as “comprising,” “including,” and “having” shall be interpreted inclusively and in an open fashion, so as not to exclude unlisted elements. Stated another way, such terms imply “not limited to” unless the context explicitly states otherwise.

The term “consisting essentially of” means that, in addition to the recited elements, what is claimed may also contain other elements (steps, structures, ingredients, components, etc.) that do not materially affect the basic and novel characteristic(s) of the claimed invention. This term excludes such other elements that materially affect the basic and novel characteristic(s) of what is claimed for its intended purpose as stated in this disclosure, even if such other elements might enhance the operability of what is claimed for some other purpose.

As used herein, the term “alkyl”, whether used alone or as part of a substituent group, includes straight hydrocarbon groups comprising from one to twenty carbon atoms. Thus, the phrase includes straight chain alkyl groups such as methyl, ethyl, propyl, butyl, pentyl, hexyl, heptyl, octyl, nonyl, decyl, undecyl, dodecyl and the like. The phrase also includes branched chain isomers of straight chain alkyl groups, including but not limited to, the following which are provided by way of example: —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)CH(CH₃)₂, —CH(CH₂CH₃)CH(CH₃)CH(CH₃)(CH₂CH₃), and others. The phrase also includes cyclic alkyl groups such as cyclopropyl, cyclobutyl, cyclopentyl, cyclohexyl, cycloheptyl, and cyclooctyl and such rings substituted with straight and branched chain alkyl groups as defined above. The phrase also includes polycyclic alkyl groups such as, but not limited to, adamantyl norbornyl, and bicyclo[2.2.2]octyl and such rings substituted with straight and branched chain alkyl groups as defined above.

As used herein, the term “alkenyl”, whether used alone or as part of a substituent group, includes an alkyl group having at least one double bond between any two adjacent carbon atoms.

As used herein, the term “alkynyl”, whether used alone or as part of a substituent group, includes an alkyl group having at least one triple bond between any two adjacent carbon atoms.

As used herein, the term “unsubstituted alkyl” refers to alkyl groups that do not contain substituents (atom or group other than hydrogen).

As used herein, the term “substituted alkyl” refers to alkyl groups as defined above in which one or more bonds to a carbon(s) or hydrogen(s) are replaced by a bond to a substituent (atom or group other than hydrogen) such as, but not limited to, an oxygen atom in groups such as hydroxy groups, alkoxy groups and aryloxy groups; a sulfur atom in groups such as, alkyl and aryl sulfide groups, sulfone groups, sulfonyl groups, and sulfoxide groups; a silicon atom in groups such as in trialkylsilyl groups, dialkylarylsilyl groups, alkyldiarylsilyl groups, and triarylsilyl groups; other heteroatoms; and other organic moieties.

As used herein, the term “unsubstituted aralkyl” refers to unsubstituted alkyl or alkenyl groups as defined above in which a hydrogen or carbon bond of the unsubstituted or substituted alkyl or alkenyl group is replaced with a bond to a substituted or unsubstituted aryl group as defined above. For example, methyl (CH₃) is an unsubstituted alkyl group. If a hydrogen atom of the methyl group is replaced by a bond to a phenyl group, such as if the carbon of the methyl were bonded to a carbon of benzene, then the compound is an unsubstituted aralkyl group (i.e., a benzyl group).

As used herein, the term “substituted aralkyl” has the same meaning with respect to unsubstituted aralkyl groups that heteroatom-substituted aryl groups had with respect to unsubstituted aryl groups. However, a substituted aralkyl group also includes groups in which a carbon or hydrogen bond of the alkyl part of the group is replaced by a bond to a non-hydrogen atom.

As used herein, the term “unsubstituted heterocyclylalkyl” refers to unsubstituted alkyl or alkenyl groups as defined above in which a hydrogen bond of the unsubstituted alkyl or alkenyl group is replaced with a bond to a substituted or unsubstituted heterocyclyl group. For example, methyl (CH₃) is a heteroatom-unsubstituted alkyl group. If a hydrogen atom of the methyl group is replaced by a bond to a heterocyclyl group, such as if the carbon of the methyl were bonded to carbon 2 of pyridine (one of the carbons bonded to the N of the pyridine) or carbons 3 or 4 of the pyridine, then the compound is an unsubstituted heterocyclylalkyl group.

As used herein, the term “substituted heterocyclylalkyl” has the same meaning with respect to unsubstituted heterocyclylalkyl groups that substituted aryl groups had with respect to unsubstituted aryl groups. However, a substituted heterocyclylalkyl group also includes groups in which a non-hydrogen atom is bonded to a heteroatom in the heterocyclyl group of the heterocyclylalkyl group such as, but not limited to, a nitrogen atom in the piperidine ring of a piperidinylalkyl group.

As used herein, the term “unsubstituted heterocyclyl” refers to both aromatic and nonaromatic ring compounds including monocyclic, bicyclic, and polycyclic ring compounds such as, but not limited to, quinuclidyl, containing 3 or more ring members of which one or more is a heteroatom such as, but not limited to, N, O, and S. Although the phrase “unsubstituted heterocyclyl” includes condensed heterocyclic rings such as benzimidazolyl, it does not include heterocyclyl groups that have other groups such as alkyl or halo groups bonded to one of the ring members, as compounds such as 2-methylbenzimidazolyl are “substituted heterocyclyl” groups as defined below.

As used herein, the term “substituted heterocyclyl” has the same meaning with respect to unsubstituted heterocyclyl groups that substituted alkyl groups had with respect to unsubstituted alkyl groups. However, a substituted heterocyclyl group also includes heterocyclyl groups in which one of the carbons is bonded to one of the non-carbon or non-hydrogen atom, such as, but not limited to, those atoms described above with respect to a substituted alkyl and heteroatom-substituted aryl groups and also includes heterocyclyl groups in which one or more carbons of the heterocyclyl group is bonded to a substituted and/or unsubstituted alkyl, alkenyl or aryl group as defined herein. This includes bonding arrangements in which two carbon atoms of a heterocyclyl group are bonded to two atoms of an alkyl, alkenyl, or alkynyl group to define a fused ring system. Examples, include, but are not limited to, 2-methylbenzimidazolyl, 5-methylbenzimidazolyl, 5-chlorobenzthiazolyl, 1-methyl piperazinyl, and 2-chloropyridyl among others.

As used herein, the term “de-acylation” refers to the removal of an acyl group, and includes de-esterification and de-amidation. Acyl groups include ester and amide groups with regard to de-acylation.

### B. Methods

Methods of deacylating acylated carbohydrates are provided with numerous advantages over existing methods. These advantages include mild reaction temperatures, mild reaction pH, and few or no hazardous byproducts (although not all embodiments of the methods described herein will have all of these advantages, and the disclosure of these advantages is not intended to limit the invention to embodiments with any such advantages). In this context, a “carbohydrate” refers to a compound containing at least one carbohydrate moiety. Thus, carbohydrate compounds include such classes of compound as monosaccharides, disaccharides, polysaccharides, glycosides, glycosamines, nucleosides, nucleoside analogs, nucleotides, and nucleotide analogs.

In some embodiments, the methods provided herein are performed with water as a solvent. Water is a particularly advantageous solvent due to its biocompatibility and minimal adverse environmental impact as compared to organic solvents. However, water was believed to be detrimental to the stability of the reaction products described herein. It was therefore surprisingly discovered that the presence of water in the reaction mixture did not substantially degrade the reaction product and provided the additional benefit of significantly increasing reaction speed. See, e.g., FIG. 85A (exemplary reaction rates in methanol only) and FIG. 85B (exemplary reaction rates in a 1:1 mixture of water and methanol).

A method of deacylating an acylated carbohydrate is provided. In a general embodiment, the method comprising mixing the acylated carbohydrate, such as a carbohydrate ester or an amidated carbohydrate, with a basic amino acid and methanol, which results in the methanol to reacting with the acyl group, such as the ester or the amide. Some such embodiments of this method proceed adequately at a temperature of about room temperature or higher. Thus, a deacylated carbohydrate compound is produced from a corresponding acylated carbohydrate.

Some embodiments of the method can be used to produce a composition of a basic amino acid and a carbohydrate compound, i.e., a deacylated carbohydrate. In such embodiments there will typically be remaining byproducts, such as the basic amino acid (e.g., lysine), methanol, a methyl ester, or any combination of the foregoing. In a further step most of the remaining basic amino acid (e.g., lysine), methanol and methyl ester may be removed. In more specific embodiments, the molar fraction of methyl esters, basic amino acid (e.g., lysine), and/or methanol that is removed is independently chosen from at least 50%, 55%, 60%, 65%, 70%, 75%, 80%, 85%, 90%, 91%, 92%, 93%, 94%, 95%, 96%, 97%, 98%, and 99%. In a specific embodiment at least one of the methanol, basic amino acid (e.g., lysine), and methyl esters are removed such that there is no detectable residue of them in the composition. In some embodiments, the basic amino acid is in the form of a polymer as described herein, e.g., poly-lysine.

In some embodiments of the method, the basic amino acid is lysine, and thus α- or ε—N-lysine-acetamide (α- or ε—N—Ac-lysine) may be produced as a byproduct. However, the level of α- or ε—N—Ac-lysine in many embodiments of the method is relatively low. In some embodiments of the method the composition contains no more than 250, 240, 230, 220, 210, 200, 190, 180, 170, 160, 150, 140, 130, 120, 110, 100, 90, 80, 70, 60, 50, 45, 40, 35, 30, 25, 20, 15, 10, 9, 8, 7, 6, 5, 4, 3, 2, and 1 ppm w/v α- or ε—N—Ac-lysine. A specific embodiment of the method produces less than 25 ppm w/v of α- or ε—N—Ac-lysine. A further specific embodiment of the method produces less than 15 ppm w/v of α- or ε—N—Ac-lysine. A further specific embodiment of the method produces no detectable α- or ε—N—Ac-lysine. Some embodiments of the method result in a level of α- or ε—N—Ac-lysine that is safe for human or animal consumption. In embodiments where α- or ε—N—Ac-lysine is produced, the method may comprise an additional step to remove α- or ε—N—Ac-lysine.

In certain embodiments, the basic amino acid, e.g., lysine, is a catalyst for the deacylation reaction. In such embodiments, α- or ε—N—Ac-lysine is not formed, and the lysine may be recycled for a further reaction in the same reaction mixture, or the lysine may be removed from the reaction mixture and utilized in another reaction, or the lysine may be retained in the resulting product mixture. In some embodiments, the resulting product mixture forms a composition for human or animal consumption, and the basic amino acid, e.g., lysine, serves as a nutritional supplement in the composition. In some embodiments, the basic amino acid is in the form of a polymer as described herein, e.g., poly-lysine, and the polymer may be removed by filtration as further described herein.

In some embodiments of the method, methyl acetate or methyl acylate may be produced as a byproduct. In some embodiments of the method the composition contains no more than 250, 240, 230, 220, 210, 200, 190, 180, 170, 160, 150, 140, 130, 120, 110, 100, 90, 80, 70, 60, 50, 45, 40, 35, 30, 25, 20, 15, 10, 9, 8, 7, 6, 5, 4, 3, 2, and 1 ppm w/v methyl acetate or methyl acylate. In a further specific embodiment of the method, the composition contains no detectable methyl acetate or methyl acylate. Some embodiments of the method result in a level of methyl acetate or methyl acylate that is safe for human or animal consumption. In embodiments where methyl acetate or methyl acylate is produced, the method may comprise an additional step to remove methyl acetate or methyl acylate.

The method disclosed herein produces no significant amount of acetamide (e.g., N-lysine acetamide) as a byproduct, and therefore has the advantage of increased safety for human and animal consumption, due to acetamide's status as a possible carcinogen. In some embodiments of the method the composition contains no more than 250, 240, 230, 220, 210, 200, 190, 180, 170, 160, 150, 140, 130, 120, 110, 100, 90, 80, 70, 60, 50, 45, 40, 35, 30, 25, 20, 15, 10, 9, 8, 7, 6, 5, 4, 3, 2, and 1 ppm w/v acetamide A specific embodiment of the method produces less than 25 ppm w/v of acetamide. A further specific embodiment of the method produces less than 15 ppm w/v of acetamide. A further specific embodiment of the method produces no detectable acetamide. Some embodiments of the method result in a level of acetamide that is safe for human or animal consumption. Such embodiments of the method may comprise an additional step to remove excess acetamide.

The acylated carbohydrate contains an acyl group on the carbohydrate group. The carbohydrate moiety can be a substituted or unsubstituted CₙH₂ₙOₙ aldoses and ketoses such as those, tetrose, pentose, hexose, heptose, octose, or larger carbohydrates, or part of more complex molecules such as glycosides and nucleosides. Examples of possible acylated pentosides are acylated D-arabinoside, D-lyxoside, D-riboside, D-xyloside, L-arabinoside, L-lyxoside, L-riboside, L-xyloside, D-ribuloside, D-xyluloside, L-ribuloside, and L-xyluloside. In some embodiments, the acylated carbohydrate is a pentose polyester. In further embodiments, the acylated carbohydrate is a pentose monoester. In a specific embodiment, the carbohydrate group is a D-riboside moiety.

In some embodiments of the method, the acylated carbohydrate is an amidated carbohydrate and contains a heteroaromatic amide. The heteroaromatic amide can be an acylated form of a nucleobase, or a hetero-arylamine.

In some embodiments of the method, the acylated carbohydrate contains a heteroaromatic ester. The heteroaromatic ester can be an acylated form of a nucleobase, or a hetero-arylamine.

In some embodiments of the method the acyl group is an alkyl ester or amide. In further embodiments of the method the acyl group is a heteroatom-substituted alkyl, heteroatom-substituted alkenyl, substituted alkynyl, substituted aralkyl, substituted heterocyclylalkyl, substituted heterocyclyl, heteroatom-unsubstituted alkyl, heteroatom-unsubstituted alkenyl, unsubstituted alkynyl, unsubstituted aralkyl, unsubstituted heterocyclylalkyl, and unsubstituted heterocyclyl. In some embodiments the ester a C₁-C₁₀ ester. In more specific embodiments the acyl group is an ethyl substituted ester. In more specific embodiments of the methods the acylated carbohydrate is an acetyl ester or amide. Such acetyl esters or amides may have varying degrees of acetylation, such as mono-acetylated, di-acetylated, or tri-acetylated. Such mono-, di-, and tri-acetylated acylated carbohydrates are in some embodiments, e.g., where the carbohydrate comprises at least 5 carbons, acetylated at one or more of the 2, 3′, and 5′ carbons.

The method can be applied to various types of acylated carbohydrates, such as esters and amides. These include acyls of glycosamines, nucleosides, nucleoside analogues and derivatives, nucleotides, nucleotide analogues, pyridinium nucleosides, pyrimidine nucleosides, purine nucleosides, nicotinamide glycosides, fructosides, galactosides, glucosides, glucuronides, rhamnosides, ribosides, ribofuranoses, D-ribofuranoses, and R-ribofuranose. In a specific embodiment of the method the acylated carbohydrate is an acyl of NR; this embodiment has the advantage of producing NR, which is a commercially valuable product. In another specific embodiment of the method, the acylated carbohydrate is an acyl of NRH, which is also a commercial valuable product. In such embodiments various acyl groups may be used, such as acetyl esters of NR and NRH. In a more specific embodiment of the method the acyl group is a triacetyl ester of NR or NRH.

In a specific embodiment of the method the acylated carbohydrate is an N-acylated purine nucleoside. In another embodiment of the method the acylated carbohydrate is an N-acylated pyrimidine nucleoside.

The molar concentration of methanol relative to the molar concentration of the acylated carbohydrate will influence the rate and yield of the reaction. In some embodiments of the method the molar concentration of methanol is advantageously at least the molar concentration of acyl groups in the acylated carbohydrate; such embodiments are expected to have the advantage of providing enough methanol groups to fully react with all of the acyl groups. In further embodiments of the method the molar concentration of methanol exceeds the molar concentration of acyl groups in the acylated carbohydrate. If the acylated carbohydrate compound contains additional acyl groups bound to parts of the molecule other than the glycosyl group, then the molar concentration of methanol may be equal to or greater than the molar concentration of only the acyl groups bound to the glycosyl group.

The reaction may be conducted at a temperature suitable to accomplish the desired reaction rate and to provide the desired level of stability to the reaction products. Some embodiments of the reaction can be conducted at relatively high temperatures. In some embodiments of the method, the reaction is allowed to take place at a temperature greater than or about 4° C. In further embodiments of the method the reaction is allowed to take place at a temperature greater than or about 20° C. In further embodiments of the method the reaction is allowed to take place at a temperature up to 50° C. In a more specific embodiment of the method the reaction is allowed to take place at 40-50, or 45-50° C. In further embodiments of the method the reaction is allowed to take place at about room temperature or higher. In further embodiments of the method the reaction is allowed to take place at about room temperature. In still further embodiments of the method the reaction is allowed to take place at a temperature greater than or about 40° C. These temperatures have the advantage of fast reaction rates and acceptable levels of stability.

In some embodiments, the method is performed at a temperature that allows the reaction to proceed to completion in a desirable timeframe while preventing degradation of the reaction products. As discussed herein, reaction speed is significantly increased when the method is performed with water as solvent (or a component of a solvent), as compared to an organic solvent. Without being bound by theory, the presence of water may contribute to the increased reaction speed by stabilizing the carbonyl intermediate and/or solubilizing the product, thereby favoring the reaction towards product formation. The increased reaction speed advantageously allows the reaction temperature to be decreased, such that the reaction still proceeds to completion while minimizing degradation. Accordingly, in certain embodiments, the method is performed at a temperature of −30° C. to about 60° C. In a further embodiment, the method is performed at a temperature of about −20° C. to about 50° C. In still further embodiments, the method is performed at a temperature of about 0° C. to about 30° C. In still further embodiments, the method is performed at a temperature of about 4° C. to about 25° C. In a specific embodiment, the method is performed at a temperature of about −20° C.

The basic amino acid has a side chain with a pKₐ of greater than 7.0. In some embodiments of the method the basic amino acid has a side chain with a pKₐ of greater than 7.5, 8.0, 8.5, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12.0, 12.1, 12.2, 12.3, 12.4, 12.5, 12.6, 12.7, 12.8, 12.9, or 13.0. In a specific embodiment the basic amino acid has a side chain with a pKₐ of greater than 10.5, such as lysine. In another specific embodiment the basic amino acid has a side chain with a pKₐ of greater than 12.4, such as arginine.

In some cases, the basic amino acid has a side chain with a high enough pKₐ to cause the pI of the amino acid to be greater than 7.0. In some cases, the basic amino acid has a side chain with a high enough pKₐ to cause the pI of the amino acid to be greater than 8.0, 8.5, 9.0, 9.1, 9.2, 9.3, 9.4, 9.5, 9.6, 9.7, 9.8, 9.9, 10.0, 10.1, 10.2, 10.3, 10.4, 10.5, 10.6, 10.7, 10.8, 10.9, or 11.0. In another specific embodiment the basic amino acid has a side chain with a high enough pKₐ to cause the pI of the amino acid to be greater than 8.0. In another specific embodiment, the basic amino acid has a side chain with a high enough pKₐ to cause the pI of the amino acid to be greater than 9.4, such as lysine. In another specific embodiment, the basic amino acid has a side chain with a high enough pKₐ to cause the pI of the amino acid to be greater than 10.7, such as arginine. The basic amino acid may be any with the properties described above, including the 20 “standard” proteogenic amino acids and nonstandard amino acids. In a specific embodiment of the method, the basic amino acid is selected from lysine and arginine. In a further embodiment, the basic amino acid is poly-lysine.

In some embodiments, the basic amino acid is in the form of a polymer, e.g., comprising monomeric basic amino acid residues. In further embodiments, the polymeric basic amino acid monomeric is formed by coupling or crosslinking basic amino acid residues to a polymer, e.g., a resin such as agarose or a copolymer compound such as divinylbenzene. In embodiments, the basic amino acid is poly-lysine. In some embodiments, the poly-lysine comprises a polymer of lysine residues, which may be polymerized at the α-carbon or the ε-carbon to form α-polylysine or ε-polylysine, respectively. α-polylysine may be comprised of L-lysine or D-lysine residues to form poly-L-lysine or poly-D-lysine. In one embodiment, the ε—NH₂ group of lysine catalyzes the deacylation reaction, and thus, the lysine polymer is α-polylysine. In further embodiments, the poly-lysine comprises lysine residues coupled or crosslinked to a polymer, e.g., a resin such as agarose or a copolymer compound such as divinylbenzene. Any form of poly-lysine may be used in the methods described herein, so long as an amine group (e.g., the ε—NH₂ group) is capable of catalyzing the deacylation reaction at the specified conditions (e.g., in methanol or a mixture of water and methanol). A polymeric basic amino acid, e.g., poly-lysine, is generally sufficiently large that it can be removed by filtration, which simplifies the purification of the deacylated carbohydrate produced by the method described herein. Accordingly, in embodiments, the method provided herein comprises (a) forming a reaction mixture by mixing an acylated carbohydrate with poly-lysine and methanol; (b) allowing the methanol to react with the acylated carbohydrate; and (c) removing the poly-lysine from the reaction mixture, e.g., by filtration. In a further embodiment, the poly-lysine removed during step (c) is recyclable and may be used in a further reaction, thereby reducing the amount of materials required.

The molar concentration of the basic amino acid will be sufficient to effectively catalyze the reaction. Without wishing to be bound by any hypothesis, it is believed that the basic amino acid catalyzes the transesterification of the acyl group by methanol. If that model is correct, then the method would require that the molar concentration of the basic amino acid will be sufficient to catalyze the methylation of at least some of the acyl groups by methanol. In some embodiments of the method, the basic amino acid is present at a molar concentration greater than a molar concentration of the acylated carbohydrate. In further embodiments of the method, the basic amino acid is present at a molar concentration greater than three times a molar concentration of the acylated carbohydrate. In further embodiments, the basic amino acid is present at a molar concentration greater than a molar concentration of acyl groups in the acylated carbohydrate. In embodiments where the basic amino acid is in polymer form, the molar number of monomeric units in the basic amino acid polymer is greater than a molar concentration of acyl groups in the acylated carbohydrate.

Other reaction conditions should be controlled to prevent degradation of the reaction products and maintenance of a sufficiently high reaction rate. For example, in some embodiments of the method it is advantageous to exclude oxygen in solvents to reduce the degradation of NRH to NR and use anhydrous conditions to reduce the degradation of NR to NAM. Accordingly, some embodiments of the method are conducted under conditions of low oxygen. In some such embodiments, the partial pressure of 02 is below 0.20, 0.19, 0.18, 0.17, 0.16, 0.15, 0.14, 0.13, 0.12, 0.11, 0.10, 0.09, 0.08, 0.07, 0.06, 0.07, 0.06, 0.05, 0.04, 0.03, 0.02, or 0.01 atm. In further embodiment reaction conditions are anoxic. In some embodiments of the method, it is advantageous to exclude water vapor to reduce the degradation of NR to NAM. Accordingly, some embodiments of the method are conducted under conditions of low moisture. In some such embodiments, moisture content is below 20, 19, 18, 17, 16, 15, 14, 13, 12, 11, 10, 9, 8, 7, 6, 5, 4, 3, 2, or 1 ppm w/w. In a further embodiment, reaction conditions are anhydrous. In a specific embodiment of the method, the reaction is conducted under anhydrous and anoxic conditions.

In a further embodiment of the method, the reaction mixture comprises water. As discussed herein, water was surprisingly discovered to be a suitable solvent for the reaction with minimal product degradation. In embodiments, the reaction mixture comprises methanol and water at a ratio of about 1:30 to about 1:1, or about 1:25 to about 1:1, or about 1:20 to about 1:1, or about 1:15 to about 1:1, or about 1:10 to about 1:1, or about 1:5 to about 1:1, or about 1:4 to about 1:1, or about 1:3 to about 1:1, or about 1:2 to about 1:1. In a specific embodiment, the reaction mixture comprises methanol and water at a ratio of about 1:1.

In some embodiments, the reaction mixture comprises water, e.g., methanol and water at a ratio of about 1:30 to about 1:1, and the method is performed at a temperature of about −30° C. to about 60° C. In a further embodiment, the method is performed at a temperature of about −20° C. to about 50° C. In still further embodiments, the method is performed at a temperature of about 0° C. to about 30° C. In still further embodiments, the method is performed at a temperature of about 4° C. to about 25° C. In an embodiment, the method is performed at a temperature of about −20° C. In a specific embodiment, the reaction mixture comprises methanol and water at a ratio of about 1:1, and the method is performed at a temperature of about −20° C. In embodiments, the reaction is performed in a 1:1 mixture of methanol and water at −20° C., and proceeds to completion (i.e., substantially complete deacylation of the acylated carbohydrate) in less than 5 days, e.g., in 72 hours. As mentioned herein, the reaction proceeds significantly faster in water as compared to organic solvent, which allows the reaction to be conducted at a sufficiently low temperature to avoid product degradation. In embodiments, the reaction in an organic solvent (e.g., 100% methanol) requires at least or about 5 days to complete at room temperature.

In some embodiments, the method produces at least 70%, at least 75%, at least 80%, at least 85%, at least 90%, at least 95%, or at least 99% yield of the deacylated carbohydrate. In a specific embodiment, the reaction mixture comprises methanol and water at a ratio of about 1:1, and the method is performed at a temperature of about −20° C., and the method produces at least 90% or at least 95% yield of the deacylated carbohydrate.

In a further embodiment, the disclosure provides a method of deacylating an acylated carbohydrate, comprising forming a reaction mixture by mixing the acylated carbohydrate, such as a carbohydrate ester or an amidated carbohydrate, with an amine base, such as morpholine, and methanol, which results in the methanol to reacting with the acyl group, such as the ester or the amide. In an embodiment, the amine base, e.g., morpholine, is immobilized, e.g., on a solid support, a resin such as agarose, or a copolymer such as divinylbenzene. In one embodiment, the immobilized morpholine comprises an N-vinylbenzylmorpholine-divinylbenzene copolymer. In a further embodiment, the amine base, e.g., morpholine, is in the form of a polymer, e.g., poly-morpholine. In some embodiments, immobilization of the amine base and/or the amine base being in a polymer form facilitates its removal from the reaction mixture upon reaction completion as described herein.

In some embodiments the reaction mixture consists of water and methanol at the recited ratios as solvents. In other embodiments, the reaction mixture consists essentially of water and methanol as solvents, such that other solvents can also be included, so long as the reaction proceeds to completion at the desired temperature and during the desired time period.

In some embodiments of the method, the basic amino acid and the acylated carbohydrate are actively mixed. Useful mixing methods include stirring, bead-beating, ball-milling, planetary milling, and co-extrusion.

### C. Compositions

Reaction mixtures are provided that are useful for the methods described above. The reaction mixtures comprise the acylated carbohydrate, basic amino acid, and methanol. The forms of the acylated carbohydrate and amino acid may be any that are described above as useful in the method. The concentrations of acylated carbohydrate, basic amino acid, and methanol may likewise be any that are described above as useful in the method. In a further embodiment, the reaction mixture further comprises water as described herein. In embodiments, the reaction mixture comprises methanol and water at a ratio of about 1:30 to about 1:1, or about 1:25 to about 1:1, or about 1:20 to about 1:1, or about 1:15 to about 1:1, or about 1:10 to about 1:1, or about 1:5 to about 1:1, or about 1:4 to about 1:1, or about 1:3 to about 1:1, or about 1:2 to about 1:1. In a specific embodiment, the reaction mixture comprises methanol and water at a ratio of about 1:1. The reaction mixture can also consist of water and methanol as solvents, or consist essentially of water and methanol as the solvents, as described herein.

A composition containing a carbohydrate compound is provided that is the product of any of the methods described above. The carbohydrate compound is the deacylated version of any of the acylated carbohydrates described as being useful in the method above. The composition may also comprise any basic amino acid described above as suitable for use in the method. In such versions of the composition less than all the basic amino acid has been removed from the reaction products; these embodiments have the advantage of providing a possible nutritional supplement in the form of the amino acid in combination with the carbohydrate compound. In some embodiments of the composition, all or substantially all of the methanol has been removed; these embodiments have the advantage of reducing or eliminating the toxic properties of methanol from the composition. In some embodiments of the composition, all or substantially all of the methyl ester has been removed; these embodiments have the advantage of reducing or eliminating the unwanted properties of methyl ester from the composition. Some embodiments of the composition have a level of acetamide, e.g., N-lysine acetamide, that is safe for consumption. Some embodiments of the composition contain no more than 250, 240, 230, 220, 210, 200, 190, 180, 170, 160, 150, 140, 130, 120, 110, 100, 90, 80, 70, 60, 50, 45, 40, 35, 30, 25, 20, 15, 10, 9, 8, 7, 6, 5, 4, 3, 2, and 1 ppm w/v of acetamide, e.g., N-lysine acetamide. A specific embodiment of the composition contains less than 25 ppm w/v of acetamide, e.g., N-lysine acetamide. A further specific embodiment of the composition contains less than 15 ppm w/v of acetamide, e.g., N-lysine acetamide. A further specific embodiment of the composition contains no detectable acetamide, e.g., N-lysine acetamide. Some embodiments of the composition contain a level of acetamide, e.g., N-lysine acetamide, that is safe for human or animal consumption. Such embodiments of the composition may result from an additional step to remove excess acetamide, e.g., N-lysine acetamide. As discussed above, the composition will in some embodiments have a level of methyl acetate or methyl acylate that is safe for consumption. Some embodiments of the composition contain no more than 250, 240, 230, 220, 210, 200, 190, 180, 170, 160, 150, 140, 130, 120, 110, 100, 90, 80, 70, 60, 50, 45, 40, 35, 30, 25, 20, 15, 10, 9, 8, 7, 6, 5, 4, 3, 2, and 1 ppm w/v methyl acetate or methyl acylate. A specific embodiment of the composition contains less than 25 ppm w/v of methyl acetate or methyl acylate. A further specific embodiment of the composition contains less than 15 ppm w/v of methyl acetate or methyl acylate. A further specific embodiment of the composition contains no detectable methyl acetate or methyl acylate. Some embodiments of the composition contain a level of methyl acetate or methyl acylate that is safe for human or animal consumption. Such embodiments of the composition may result from an additional step to remove excess methyl acetate or methyl acylate. As discussed above, the composition will in some embodiments have no significant amount of acetamide or a level of acetamide that is safe for consumption. In some embodiments of the composition contains no more than 250, 240, 230, 220, 210, 200, 190, 180, 170, 160, 150, 140, 130, 120, 110, 100, 90, 80, 70, 60, 50, 45, 40, 35, 30, 25, 20, 15, 10, 9, 8, 7, 6, 5, 4, 3, 2, and 1 ppm w/v acetamide. A specific embodiment of the composition contains less than 25 ppm w/v of acetamide. A further specific embodiment of the composition contains less than 15 ppm w/v of acetamide. A further specific embodiment of the composition contains no detectable acetamide. Low concentrations of acetamide have the advantage of increased safety for human and animal consumption, due to its status as a possible carcinogen. Some embodiments of the composition contain a level of acetamide that is safe for human or animal consumption. Such embodiments of the composition may result from an additional step to remove excess acetamide.

### D. Exemplary Embodiments

Embodiment 1 provides a method of deacylating an acylated carbohydrate, the method comprising: (a) forming a reaction mixture by mixing the acylated carbohydrate with a basic amino acid and methanol; and (b) allowing the methanol to react with an acyl group of the acylated carbohydrate.
Embodiment 2 provides a method of producing a deacylated carbohydrate from a corresponding acylated carbohydrate, the method comprising: (a) forming a reaction mixture by mixing the acylated carbohydrate with a basic amino acid and methanol; and (b) allowing the methanol to react with the acylated carbohydrate under conditions sufficient to form the deacylated carbohydrate.
Embodiment 3 provides a method of producing a composition comprising a basic amino acid and a deacylated carbohydrate, the method comprising: (a) forming a reaction mixture by mixing an acylated carbohydrate with a basic amino acid and methanol; (b) allowing the methanol to react with the acylated carbohydrate to form the deacylated carbohydrate and a methyl ester; and (c) removing unreacted methanol from the reaction mixture.
Embodiment 4 includes the method of embodiment 3, further comprising removing the methyl ester from the reaction mixture.
Embodiment 5 includes the method of any one of embodiments 1-4, wherein the method produces less than 25 ppm of methyl ester.
Embodiment 6 includes the method of any one of embodiments 1-5, wherein the method produces no detectable acetamide.
Embodiment 7 includes the method of any one of embodiments 1-6, further comprising removing the basic amino acid from the reaction mixture following step (b).
Embodiment 8 includes the method of any one of embodiments 1-7, wherein the acylated carbohydrate is a pentose poly- or mono-ester.
Embodiment 9 includes the method of any one of embodiments 1-7, wherein the acylated carbohydrate is mono-acetylated, di-acetylated, or tri-acetylated.
Embodiment 10 includes the method of any one of embodiments 1-9, wherein the acylated carbohydrate comprises at least 5 carbons and is acetylated at one or more of its 2′, 3′, and 5′ carbons.
Embodiment 11 includes the method of any one of embodiments 1-10, wherein the acylated carbohydrate is a substituted ester.
Embodiment 12 includes the method of embodiment 11, wherein the acylated carbohydrate is a methyl substituted ester.
Embodiment 13 includes the method of embodiment 11, wherein the acylated carbohydrate is a C1-C10 ester.
Embodiment 14 includes the method of any one of embodiments 1-10, wherein the acylated carbohydrate is an ester of nicotinamide riboside.
Embodiment 15 includes the method of embodiment 14, wherein the acylated carbohydrate is an ester of reduced nicotinamide riboside.
Embodiment 16 includes the method of any one of embodiments 1-10, wherein the acylated carbohydrate is acetylated nicotinamide riboside.
Embodiment 17 includes the method of embodiment 16, wherein the acylated carbohydrate is triacetyl nicotinamide riboside.
Embodiment 18 includes the method of any one of embodiments 1-10, wherein the carbohydrate is a riboside.
Embodiment 19 includes the method of any one of embodiments 1-10, wherein the acylated carbohydrate is a pentose heteroarylamide.
Embodiment 20 includes the method of any one of embodiments 1-10, wherein the carbohydrate is a ribofuranoside compound.
Embodiment 21 includes the method of embodiment 20, wherein the carbohydrate is a D-ribofuranoside compound.
Embodiment 22 includes the method of any one of embodiments 1-10, wherein the carbohydrate is a nucleoside.
Embodiment 23 includes the method of any one of embodiments 1-10, wherein the carbohydrate is nicotinamide riboside.
Embodiment 24 includes the method of embodiment 23, wherein the carbohydrate is reduced nicotinamide riboside.
Embodiment 25 includes the method of any one of embodiments 1-10, wherein the acylated carbohydrate is a carbohydrate ester.
Embodiment 26 includes the method of any one of embodiments 1-7, wherein the acylated carbohydrate is a carbohydrate amide.
Embodiment 27 includes the method of any one of embodiments 1-7, wherein the acylated carbohydrate is a heteroaryl acetamide compound.
Embodiment 28 includes the method of any one of embodiments 1-7, wherein the acylated carbohydrate is a benzamide compound.
Embodiment 29 includes the method of any one of embodiments 1-10, wherein the acylated carbohydrate is a furanoside, and wherein the furanoside is acetylated at one or more of its 2′, 3′, and 5′ carbons.
Embodiment 30 includes the method of any one of embodiments 1-29, wherein a molar concentration of the methanol is at least the molar concentration of acyl groups in the acylated carbohydrate.
Embodiment 31 includes the method of any one of embodiments 1-30, wherein the method is performed at a temperature greater than or equal to 20° C.
Embodiment 32 includes the method of any one of embodiments 1-31, wherein the method is performed at about 20° C. to about 25° C.
Embodiment 33 includes the method of any one of embodiments 1-30, wherein the method is performed at a temperature greater than or equal to 40° C.
Embodiment 34 includes the method of any one of embodiments 1-33, wherein the basic amino acid is lysine.
Embodiment 35 includes the method of any one of embodiments 1-33, wherein the basic amino acid is arginine.
Embodiment 36 includes the method of any one of embodiments 1-35, wherein the basic amino acid is present at a molar concentration greater than or equal to a molar volume of the acylated carbohydrate.
Embodiment 37 includes the method of embodiment 36, wherein the basic amino acid is present at a molar concentration greater than or equal to three times the molar volume of the acylated carbohydrate.
Embodiment 38 includes the method of any one of embodiments 1-37, wherein the reaction is conducted under anhydrous and anoxic conditions.
Embodiment 39 includes the method of any one of embodiments 1-37, wherein the reaction mixture further comprises water.
Embodiment 40 includes the method of embodiment 39, wherein the reaction mixture comprises methanol and water at a ratio of about 1:30 to about 1:1.
Embodiment 41 includes the method of embodiment 39 or 40, wherein the method is performed at a temperature of about −30° C. to about 60° C.
Embodiment 42 includes the method of any one of embodiments 39-41, wherein the method is performed at a temperature of about −20° C. to about 50° C.
Embodiment 43 includes the method of any one of embodiments 39-42, wherein the method is performed at a temperature of about 0° C. to about 30° C.
Embodiment 44 includes the method of any one of embodiments 39-43, wherein the method is performed at a temperature of about 4° C. to about 25° C.
Embodiment 45 includes the method of embodiment 39 or 40, wherein the reaction mixture comprises methanol and water at a ratio of about 1:1, and wherein the method is performed at about −20° C.
Embodiment 46 includes the method of any one of embodiments 39-45, wherein the method produces at least 90% yield of the deacylated carbohydrate.
Embodiment 47 includes the method of any one of embodiments 39-46, wherein the method produces at least 95% yield of the deacylated carbohydrate.
Embodiment 48 includes the method of any one of embodiments 1-47, wherein the basic amino acid catalyzes methylation of the acyl group by methanol.
Embodiment 49 includes the method of any one of embodiments 1-48, comprising mixing the acylated carbohydrate with a basic amino acid and methanol by bead-beating.
Embodiment 50 includes the method of any one of embodiments 1-48, comprising mixing the acylated carbohydrate with a basic amino acid and methanol by stirring.
Embodiment 51 includes the method of any one of embodiments 1-48, comprising mixing the acylated carbohydrate with a basic amino acid and methanol by ball-milling.
Embodiment 52 includes the method of any one of embodiments 1-48, comprising mixing the acylated carbohydrate with a basic amino acid and methanol by co-extrusion.
Embodiment 53 provides a reaction mixture for deacylation of a carbohydrate, the reaction mixture comprising: a basic amino acid, the acylated carbohydrate, and methanol.
Embodiment 54 includes the reaction mixture of embodiment 53, further comprising water.
Embodiment 55 includes the reaction mixture of embodiment 54, comprising methanol and water at a ratio of about 1:10 to about 1:1.
Embodiment 56 provides a composition comprising a basic amino acid and a deacylated carbohydrate that is the product of a process comprising: (a) forming a reaction mixture by mixing an acylated carbohydrate with a basic amino acid and methanol; (b) allowing the methanol to react with the acylated carbohydrate to form the deacylated carbohydrate and a methyl ester; and (c) removing unreacted methanol from the reaction mixture.
Embodiment 57 includes the composition of embodiment 56, wherein the process further comprises removing the methyl ester from the reaction mixture.
Embodiment 58 includes the composition of embodiment 56 or 57, comprising less than 25 ppm of acetamide.
Embodiment 59 includes the composition of any one of embodiments 56-58, comprising no detectable acetamide.
Embodiment 60 includes the composition of any one of embodiments 56-59, comprising no detectable methanol.
Embodiment 61 includes the composition of any one of embodiments 56-60, comprising no detectable methyl ester.
Embodiment 62 includes the composition of any one of embodiments 56-61, wherein the reaction mixture further comprises water.
Embodiment 63 includes the composition of embodiment 62, wherein the reaction mixture comprises methanol and water at a ratio of about 1:10 to about 1:1.
Embodiment 64 includes the composition of any one of embodiments 56-63, wherein the reaction mixture comprises methanol and water at a ratio of about 1:1, and wherein the process is performed at about −20° C.
Embodiment 65 provides a composition formed by the method of any one of embodiments 1-52.

### E. Examples

### I. Working Example I

### Application of Lysine/Methanol Mixture for the Deprotection of Other Nucleosides

A mixture of uridine-triacetate (1 equiv.) in methanol and lysine (3 equiv.) were stirred at room temperature. After the completion of the reaction, the reaction mixture was distilled off to collect the mixture of deacetylated uridine and lysine.

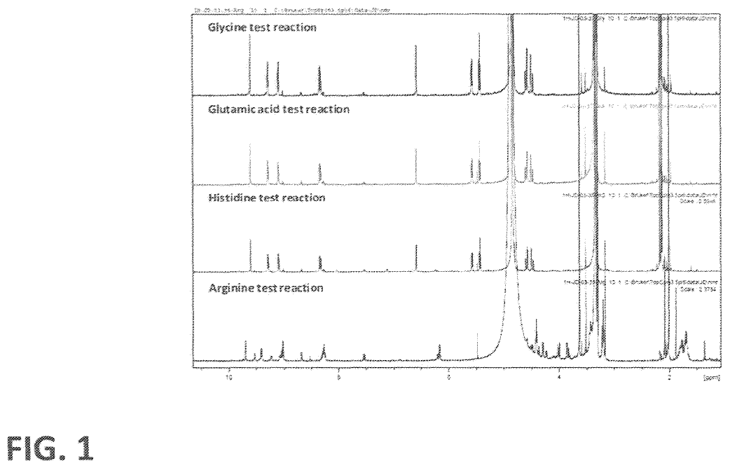

Nicotinamide riboside triacetate (NRTA) deacetylation was tested with different amino acids such as glycine, histidine, glutamic acid, and arginine in addition to lysine. While the attempts of deacetylation using glycine, glutamic acid, and histidine failed completely, arginine was found to induce partial deacetylation under the same conditions. On the other hand, lysine proved to be more effective for the deacetylation of NRTA. Given the efficiency of deacetylation, lysine was finally selected for the optimization experiments. Experiments were monitored by nuclear magnetic resonance (NMR), and the results are shown in FIG. 1.

### Deprotection/Deacetylation of NRTA to NR by Lysine

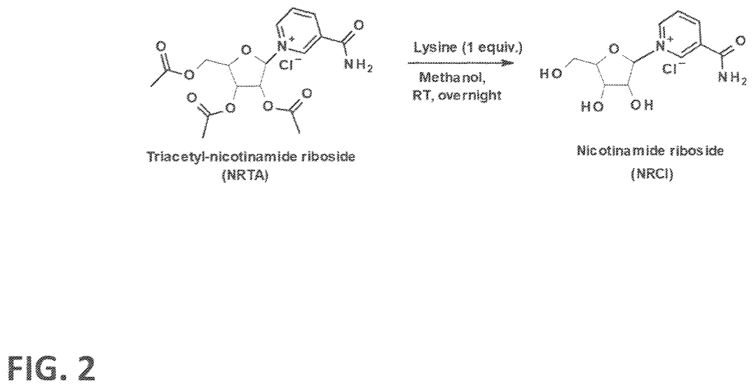

From early optimization studies, it was found that molar equivalency of lysine is sufficient to carry out the NRTA deacetylation in approximately 15-16 hours at room temperature in polar protic solvents, as shown in FIG. 2. Although in 6-7 hours, deacetylation appeared to be complete by NMR, the experiment was continued overnight at room temperature to ensure complete consumption of the starting material to NR. As any moisture can potentially be detrimental to the product formation (NR), precautions were taken to minimize or eliminate any traces of moisture by thoroughly drying the reagents and solvents (methanol) before the reaction. During the optimization studies for this Example, it was noticed that any trace amount of moisture can result in and/or enhance the degradation of NR to NAM. Considering the solubility of the reaction substrates (NRCl and lysine), methanol was selected as an ideal solvent.

### Deacetylation of NRTA Using Different Amino Acids

NRTA deacetylation was tested with different amino acids such as glycine, histidine, glutamic acid, and arginine in addition to lysine. These experiments showed lysine to be more effective for the deacetylation of NRTA, with arginine being mildly effective in carrying out the complete deacetylation of NRTA. Experiments were monitored by NMR, and the results are shown in FIG. 1.

### Procedure for the Deprotection of NRHTA Deacetylation Using Different Amounts/Equivalents of Lysine

To begin with, experiments were conducted to determine the amount of lysine required for NRH triacetate (NRHTA) deacetylation. The studies revealed that a quantity of 3 equivalents of lysine is required for the complete deacetylation of NRHTA. If the reaction were to be conducted at room temperature using 3 equivalents of lysine, then the deacetylation of NRHTA would be completed in 3 days (72 hours). However, further studies at elevated temperatures found that deacetylation can be effectively conducted at 45-50° C. without compromising the quality of the product or resulting in the formation of undesired side-products. Methanol was selected as an ideal solvent after considering the solubility of NRHTA and lysine in various solvents.

Keeping in consideration of all the above results, it was concluded that optimum conditions for NRHTA deacetylation are approximately 3 equivalents of lysine and a temperature of 45-50° C. in dry degassed methanol as a solvent.

During the studies, the efficiency of methanolysis catalyzed by lysine to carry out the debenzoylation was evaluated. It was noticed that N-benzoyl-cytidine was debenzoylated very effectively using 3 equivalents of lysine. Debenzoylation was also carried out using 1 equivalent of lysine to check the atom efficiency. Regardless of reaction (whether it is deacetylation or debenzoylation), it was noticed that use of stoichiometric amounts of lysine was inevitable and any less would only enhance the reaction times. Use of excess lysine always had a positive impact on the rate of the reaction, as increases in the lysine equivalents enhanced the rate of the reaction. A table of compounds from the study is shown in Table 1.

TABLE 1
Compounds and products for working example 1.

| Compound | Substrate | Product |
|---|---|---|
| 1 | 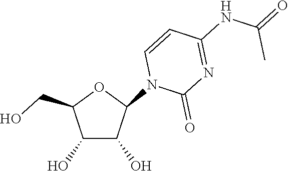 [SMILES: CC(=O)Nc1ccn([C@@H]2O[C@H](CO)[C@@H](O)[C@H]2O)c(=O)n1] N-acetyl-cytidine | 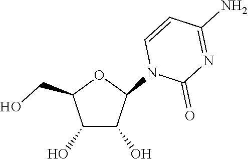 [SMILES: Nc1ccn([C@@H]2O[C@H](CO)[C@@H](O)[C@H]2O)c(=O)n1] Cytidine |
| 2 | 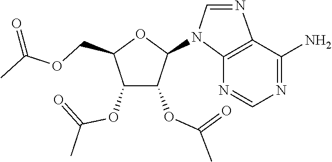 [SMILES: CC(=O)OC[C@H]1O[C@@H](n2cnc3c(N)ncnc32)[C@H](OC(C)=O)[C@@H]1OC(C)=O] | 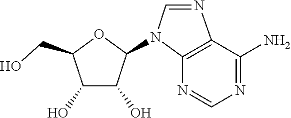 [SMILES: Nc1ncnc2c1ncn2[C@@H]1O[C@H](CO)[C@@H](O)[C@H]1O] Adenosine |
| | Triacetyl-adenosine | |
| 3 | 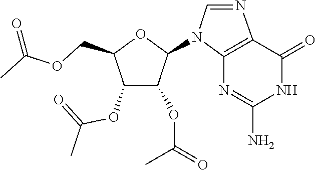 [SMILES: CC(=O)OC[C@H]1O[C@@H](n2cnc3c(=O)[nH]c(N)nc32)[C@H](OC(C)=O)[C@@H]1OC(C)=O] | 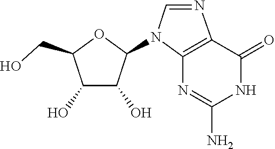 [SMILES: Nc1nc2c(ncn2[C@@H]2O[C@H](CO)[C@@H](O)[C@H]2O)c(=O)[nH]1] Guanosine |
| | Triacetyl-guanosine | |
| 4 | 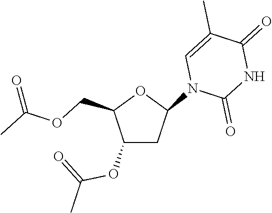 [SMILES: CC(=O)OC[C@H]1O[C@@H](n2cc(C)c(=O)[nH]c2=O)C[C@@H]1OC(C)=O] | 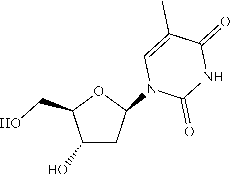 [SMILES: Cc1cn([C@H]2C[C@H](O)[C@@H](CO)O2)c(=O)[nH]c1=O] Thymidine |
| | 3′,5′-diacetyl-thymidine | |
| 5 | 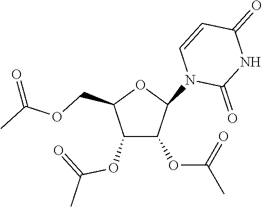 [SMILES: CC(=O)OC[C@H]1O[C@@H](n2ccc(=O)[nH]c2=O)[C@H](OC(C)=O)[C@@H]1OC(C)=O] | 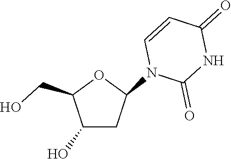 [SMILES: O=c1ccn([C@H]2C[C@H](O)[C@@H](CO)O2)c(=O)[nH]1] Uridine |
| | Triacetyl-uridine | |
| 6 | 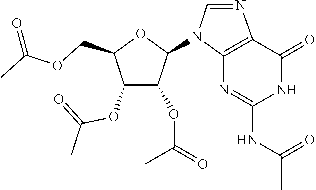 [SMILES: CC(=O)Nc1nc2c(ncn2[C@@H]2O[C@H](COC(C)=O)[C@@H](OC(C)=O)[C@H]2OC(C)=O)c(=O)[nH]1] | 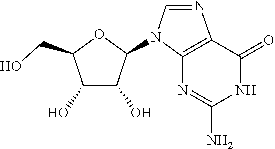 [SMILES: Nc1nc2c(ncn2[C@@H]2O[C@H](CO)[C@@H](O)[C@H]2O)c(=O)[nH]1] Guanosine |
| | N-acetyl-2′,3′,5′-tri-O-acetyl-guanosine | |
| 7 | 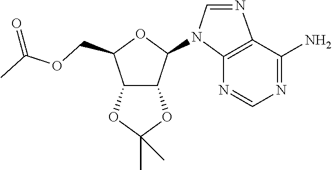 [SMILES: CC(=O)OC[C@H]1O[C@@H](n2cnc3c(N)ncnc32)[C@@H]2OC(C)(C)O[C@@H]21] | 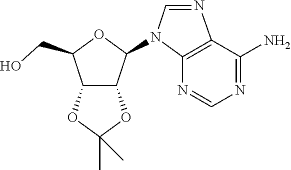 [SMILES: CC1(C)O[C@@H]2[C@H](O1)[C@@H](CO)O[C@H]2n1cnc2c(N)ncnc21] |
| | 5′-Acetyl-2′,3′-isopropylidene-adenosine | 2′,3′-isopropylidene-adenosine |
| 8 | 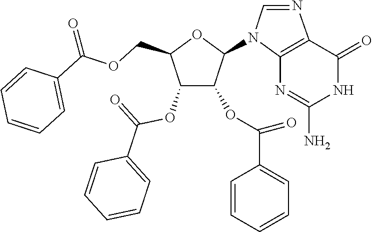 [SMILES: Nc1nc2c(ncn2[C@@H]2O[C@H](COC(=O)c3ccccc3)[C@@H](OC(=O)c3ccccc3)[C@H]2OC(=O)c2ccccc2)c(=O)[nH]1] | 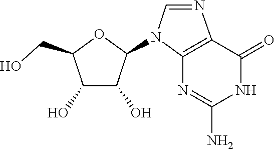 [SMILES: Nc1nc2c(ncn2[C@@H]2O[C@H](CO)[C@@H](O)[C@H]2O)c(=O)[nH]1] Guanosine |
| | 2′,3′,5′-tri-O-benzoyl-guanosine | |
| 9 | 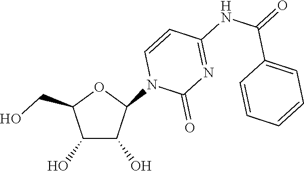 [SMILES: O=C(Nc1ccn([C@@H]2O[C@H](CO)[C@@H](O)[C@H]2O)c(=O)n1)c1ccccc1] N-benzoyl-cytidine | 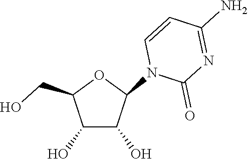 [SMILES: Nc1ccn([C@@H]2O[C@H](CO)[C@@H](O)[C@H]2O)c(=O)n1] Cytidine |

### General Procedure for the Deacetylation of Nucleosides

The above methodology was extended to other ribose derivatives and a variety of purine and pyrimidine derivatives were deacetylated to render free nucleosides by mere use of lysine and methanol. To a stirred solution of the corresponding acetylated nucleoside (1 equiv.) was added lysine (1-3 equiv.), followed by the addition of anhydrous methanol. The resulting mixture was stirred at 50-70° C. until the complete conversion/deacetylation of the starting material. After the reaction was complete, desired compound was obtained by recrystallization using a mixture of ether and methanol. Solid obtained was dried thoroughly to remove traces of methyl acetate obtained as a side-product and the residual solvent traces. The resulting dry compound was analyzed by ¹HNMR, ¹³CNMR and ESI mass spectrometry. For cytidine: ¹H NMR (400 MHz, D₂O) ppm: 3.67-3.71 (dd, 2H, J=4.4 Hz, 2.8 Hz), 3.99-4.02 (in, 1H), 4.07-4.10 (t, 1H, J=5.8 Hz), 4.17-4.20 (m, 1H), 5.77 (d, 1H, J=4.0 Hz), 5.93 (d, 1H, 7.5 Hz), 7.72 (d, 1H, J=7.5 Hz). ¹³CNMR (100 MHz, D₂O) ppm: 60.67, 69.2, 73.8, 83.7, 90.2, 96.1, 141.6, 157.6, 166.1. For triacetyl guanosine: ¹H NMR (400 MHz, CDCl₃) ppm: ¹³CNMR (100 MHz, CDCl₃) ppm: 20.6, 20.8, 20.9, 63.5, 70.7, 72.4, 79.9, 84.8, 117.2, 136.0, 151.5, 154.3, 157.0, 169.7, 169.8, 170.5; Calculated Mass: 409.12; Observed Mass: 409.87; Molecular Formula: C₁₆H₁₉N₅O₈; (See, for example: Journal of the American Chemical Society (2005), 127(51), 18133-18142, “A Quadruply Hydrogen Bonded Hetero-Complex Displaying High-Fidelity Recognition”, which is incorporated by reference in its entirety herein). For tetra-acetyl guanosine: ¹H NMR (400 MHz, D₂O) ppm: ¹³CNMR (100 MHz, CDCl₃) ppm: 20.3, 20.5, 20.8, 24.2, 63.1, 70.9, 72.7, 80.0, 87.4, 122.5, 138.4, 147.50, 147.53, 155.4, 169.4, 169.6, 171.4, 172.1; Calculated Mass (M+1): 452.14; Observed Mass: 452.01; Molecular Formula: C₁₈H₂₂N₅O₉. For guanosine: ¹H NMR (400 MHz, DMSO) ppm: ¹³CNMR (100 MHz, DMSO) ppm: 61.8, 70.8, 74.09, 85.62, 86.79, 117.16, 135.90, 151.74, 154.33, 157.

### Selection of Lysine Over Other Amino Acids for Deacetylation

NRTA deacetylation was tested with different amino acids such as glycine, histidine, glutamic acid, and arginine in addition to lysine. While the attempts of deacetylation using glycine, glutamic acid and histidine failed completely, arginine was found to induce partial deacetylation under the same conditions. Lysine on the other hand, proved to be more effective for the deacetylation of NRTA. Given the efficiency of deacetylation, lysine was finally selected for the optimization experiments. Experiments were monitored by NMR and the results are shown in FIG. 1.

For some of the nucleosides (e.g. acetylated uridine), deacetylation was found to be accomplished at room temperature, but the conversion was lengthy, compared to those that were conducted at 50-60° C. In some cases, the expected product did not form at all when the reaction was conducted at room temperature.

### Deprotection/Deacetylation of NRTA to NR by Lysine

From the optimization studies, it was found that 1 equivalent of lysine is sufficient to carry out the NRTA deacetylation in approximately 15-16 h at room temperature in methanol. Although in 6-7 hours, deacetylation appeared to be complete by NMR, the experiment was continued overnight at room temperature to ensure complete consumption of the starting material to NR. As any moisture can potentially be detrimental to the product formation (NR), precautions were taken to minimize or eliminate any traces of moisture by thoroughly drying the reagents and solvents (methanol) before the reaction. During optimization studies for this Example, it was noticed that any trace amount of moisture can result in and/or enhance the degradation of NR to NAM. Considering the solubility of the reaction substrates (NRCl and lysine), methanol was selected as an ideal solvent.

### II. Working Example II

### Methods and Reagents

Nuclear magnetic resonance (NMR) spectra were recorded on a Bruker Avance III HD 400 spectrometer (¹H, 400.11 and ¹³C, 100.62 MHz) using residual proton signal (¹H) and that of carbon atom (¹³C) of a deuterated solvent as an internal standard relative to TMS. Column chromatography was performed on silica gel columns using medium pressure liquid chromatography systems (Teledyne) with UV monitoring of eluted fractions (at 280 nm and 350 nm). Analytical TLCs were performed with Merck silica gel 60 F254 plates; visualization of TLCs was accomplished by UV light. High-resolution mass spectrometry (HRMS) spectra were obtained on a LTQ Orbitrap XL Mass Spectrometer (Heated Electrospray (HESI) source, positive polarity, capillary temp 200° C., source voltage 3.0 kV). Amberlite IRA-410, Cl-form, ion exchange resin was purchased from Acros Organics.

All commercial reagents and solvents were purchased from VWR and Oakwood chemicals and used without further purification. Anhydrous dichloromethane (DCM) was obtained leaving DCM over calcinated molecular sieves for more than 72 hours.

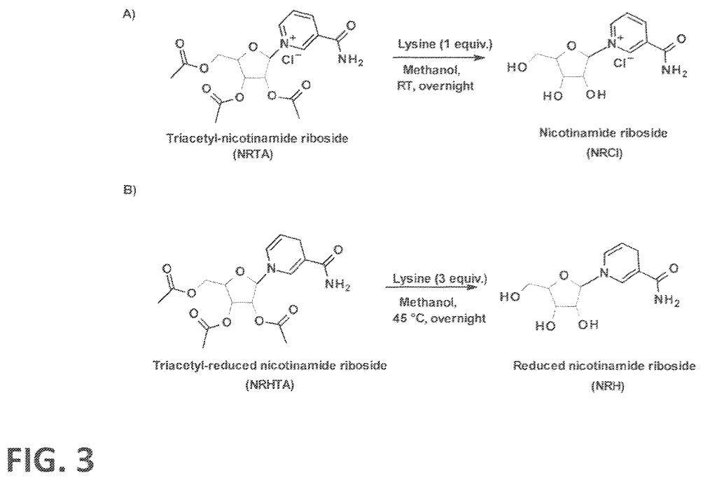

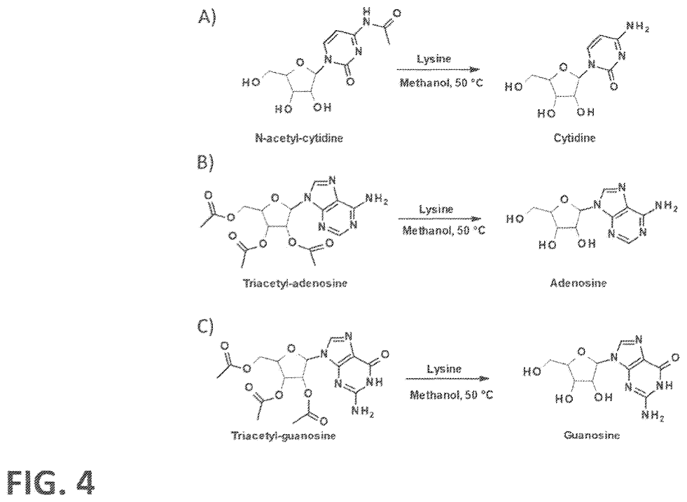

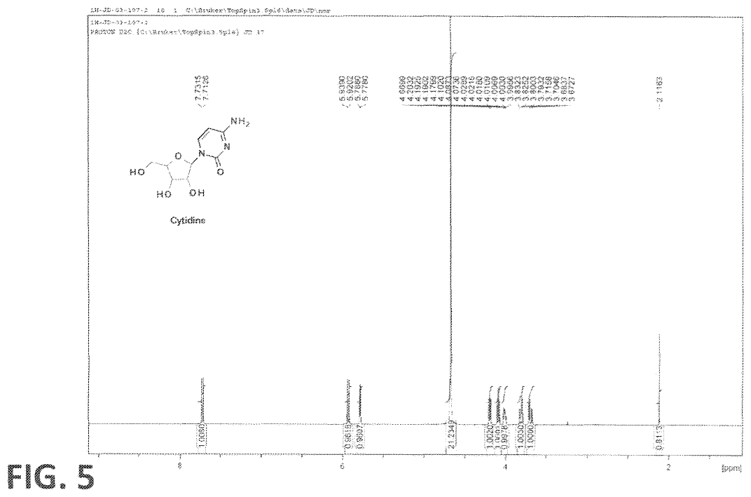

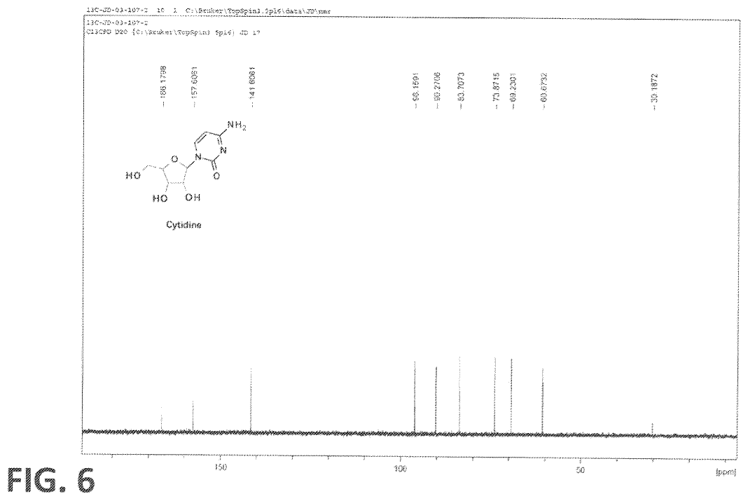

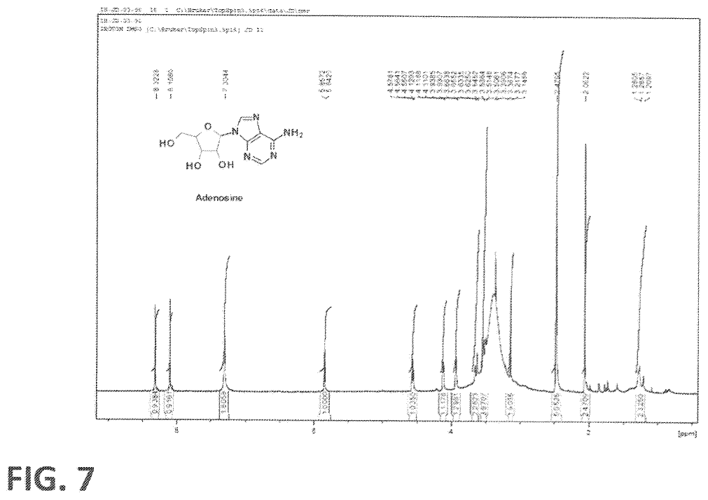

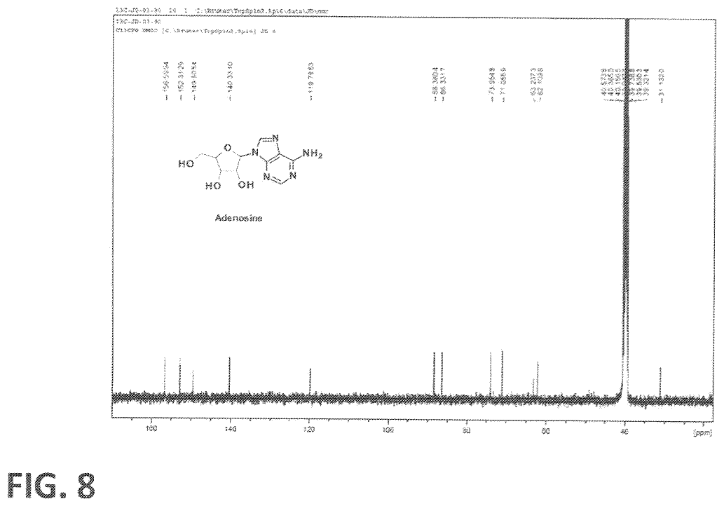

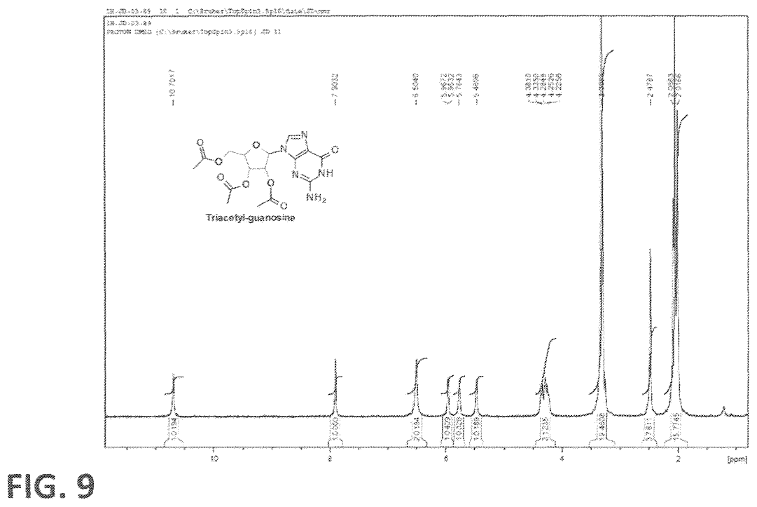

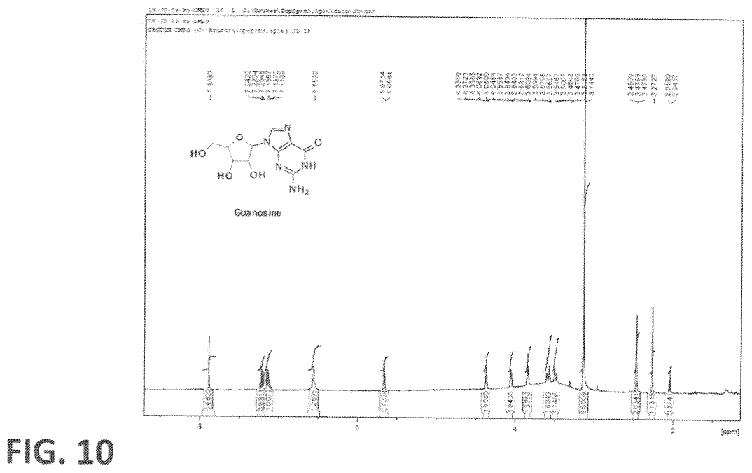

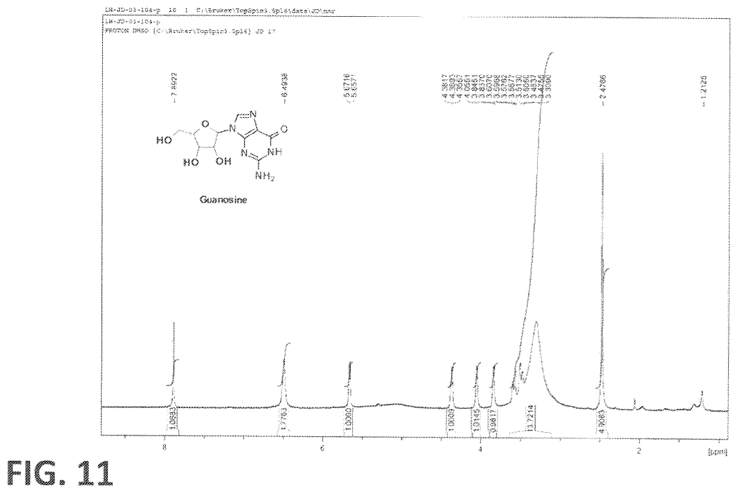

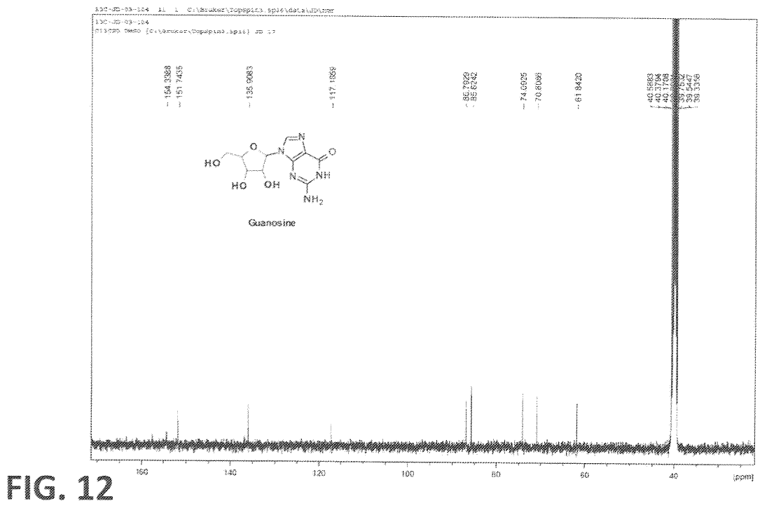

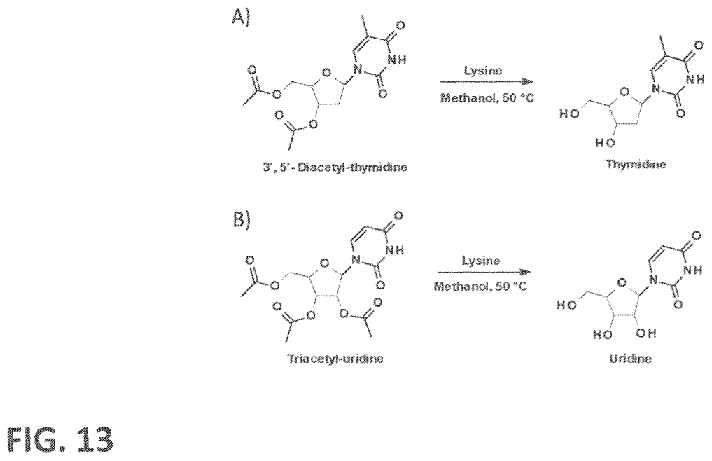

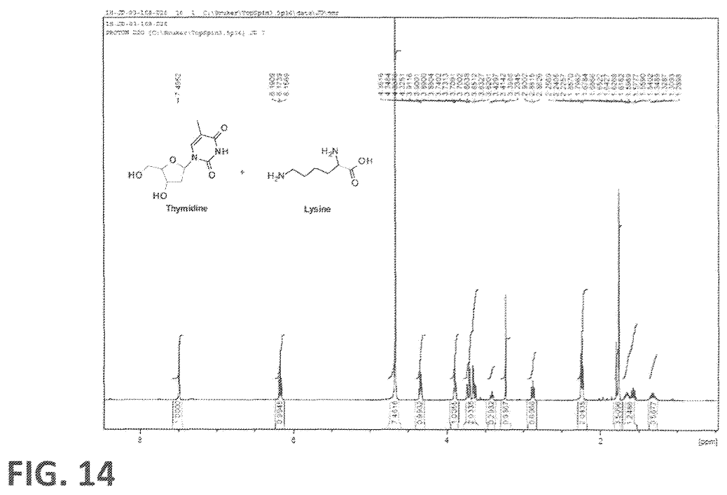

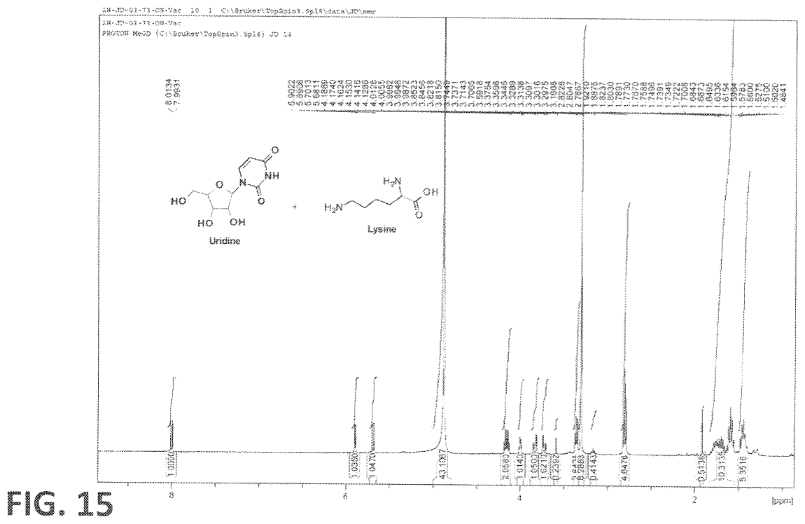

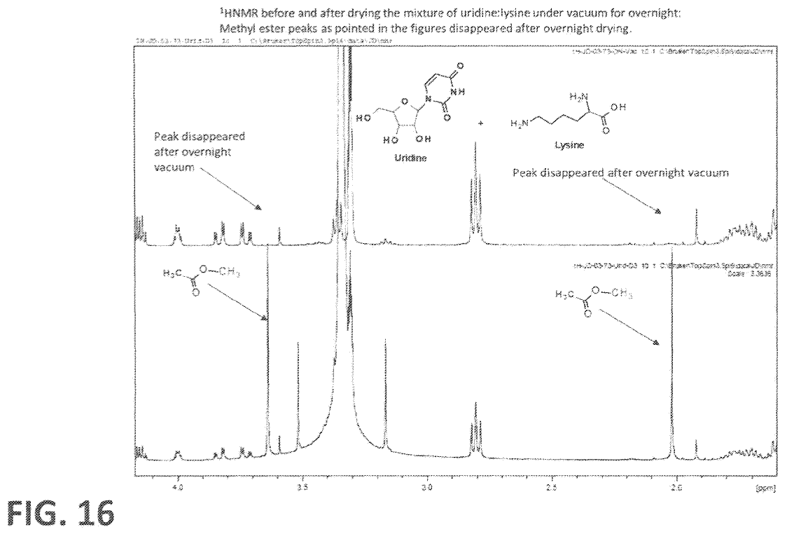

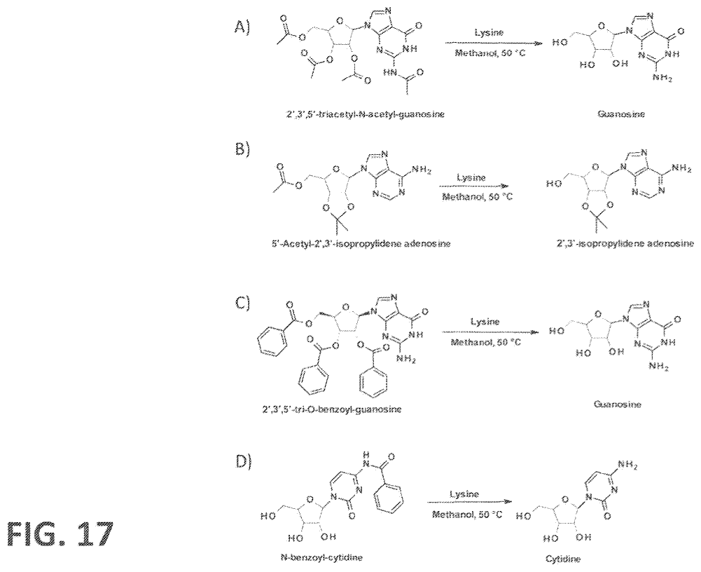

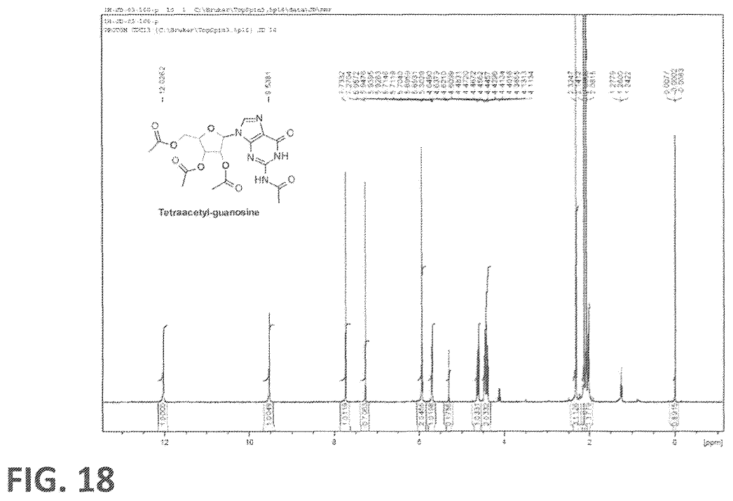

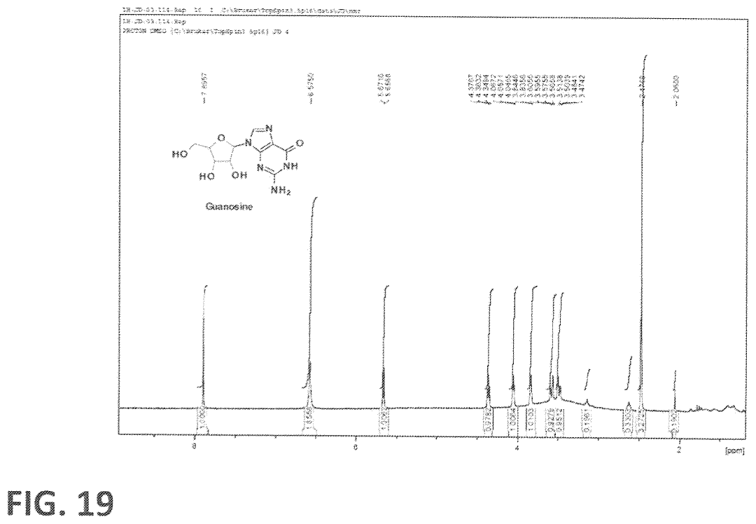

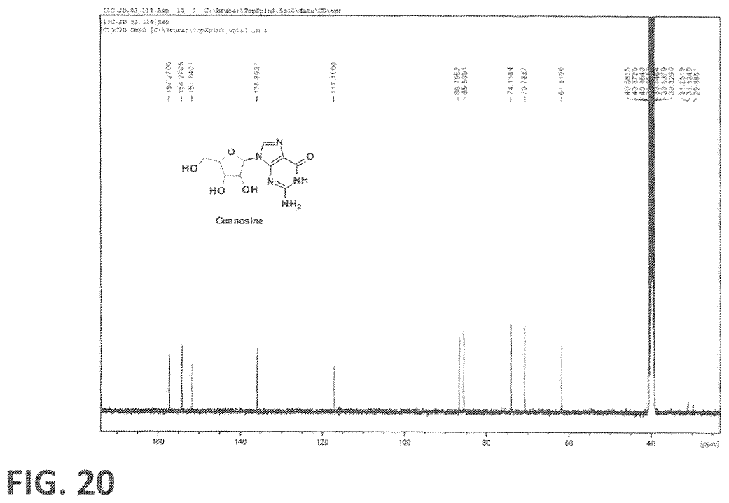

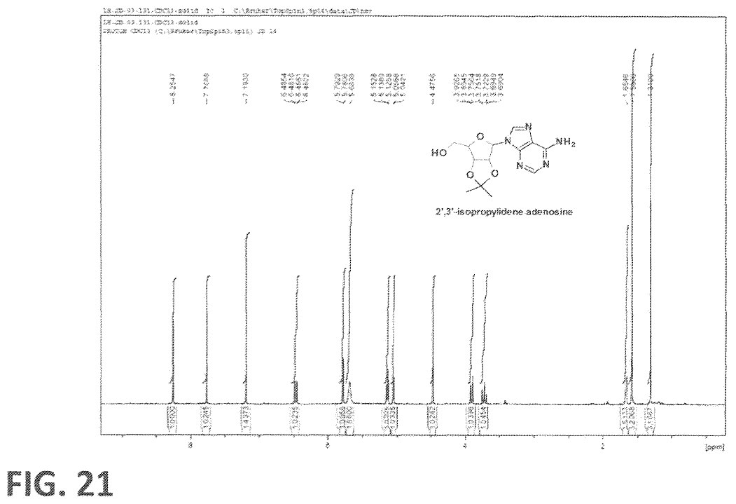

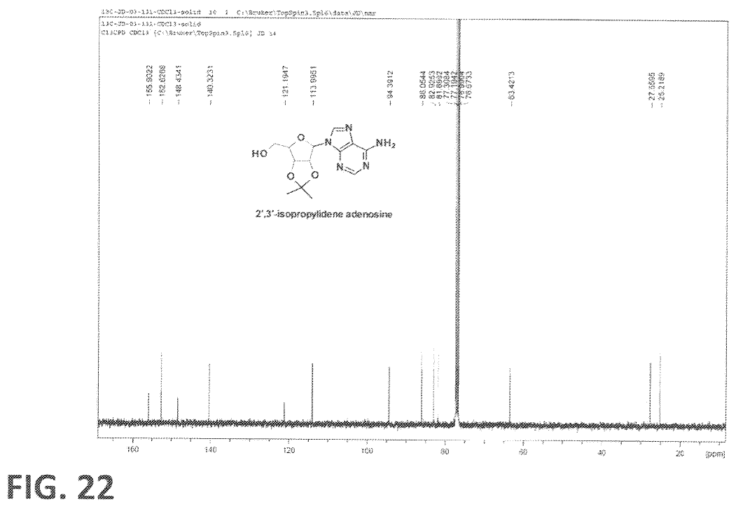

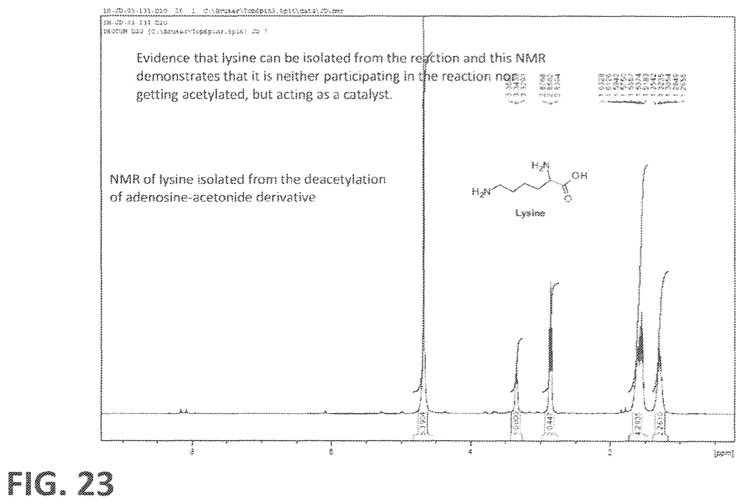

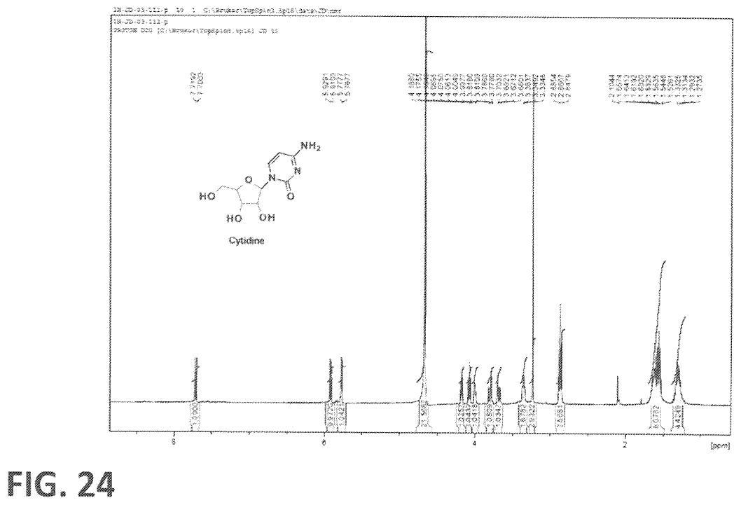

Schematics for the deacetylation of nicotinamide riboside triacetate (NRTA) are shown in FIG. 3A, and for the deacetylation of reduced nicotinamide riboside triacetate (NRHTA) are shown in FIG. 3B. Schematics and NMR spectra for the facile deprotection of other nucleosides using lysine are shown in FIG. 4A, FIG. 4B, FIG. 4C, FIG. 5, FIG. 6, FIG. 7, FIG. 8, FIG. 9, FIG. 10, FIG. 11, FIG. 12, FIG. 13A, FIG. 13B, FIG. 14, FIG. 15, FIG. 16, FIG. 17A, FIG. 178, FIG. 17C, FIG. 17D, FIG. 18, FIG. 19, FIG. 20, FIG. 21, FIG. 22, FIG. 23, and FIG. 24.

### Selection of Lysine Over Other Amino Acids for Deacetylation

NRTA deacetylation was tested with different amino acids such as glycine, histidine, glutamic acid and arginine in addition to lysine. While the attempts of deacetylation using glycine, glutamic acid and histidine failed completely, arginine was found to induce partial deacetylation under the same conditions. Lysine on the other hand, proved to be more effective for the deacetylation of NRTA. Given the facile reactivity of lysine, it became the preferred option for the deacetylation experiments. Experiments were monitored by NMR and the results are shown in FIG. 1.

### Optimization of Reaction Conditions for the NRTA Deacetylation

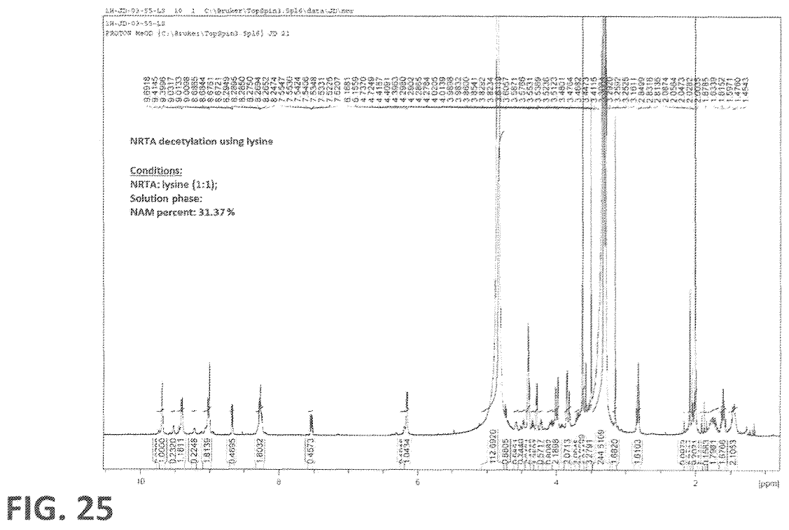

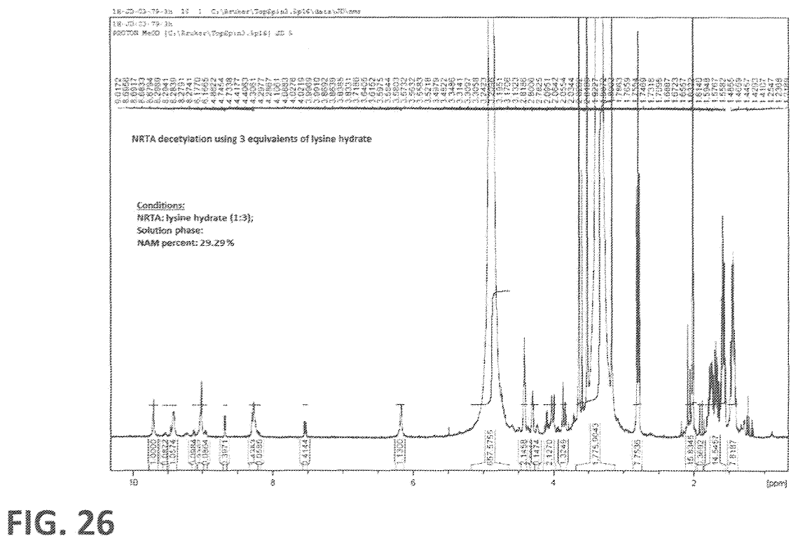

Initial studies on NRTA deacetylation were conducted using lysine hydrate. Since the presence of moisture was believed to be detrimental to the product formation, lysine (97% purity from Oakwood) was used. It is to be noted that there was no significant difference in the deacetylation studies conducted using lysine hydrate and lysine (97% purity). NRTA deacetylation using lysine is shown in FIG. 25, while NRTA deacetylation using 3 equivalents of lysine hydrate is shown in FIG. 26.

During the optimization studies, it was found that 1 equivalent of lysine is sufficient to carry out the NRTA deacetylation in approximately 15-16 hours at room temperature. Although in 6-7 hours, deacetylation appeared to be complete by NMR, the experiment was continued overnight at room temperature to ensure complete consumption of the starting material to NR.

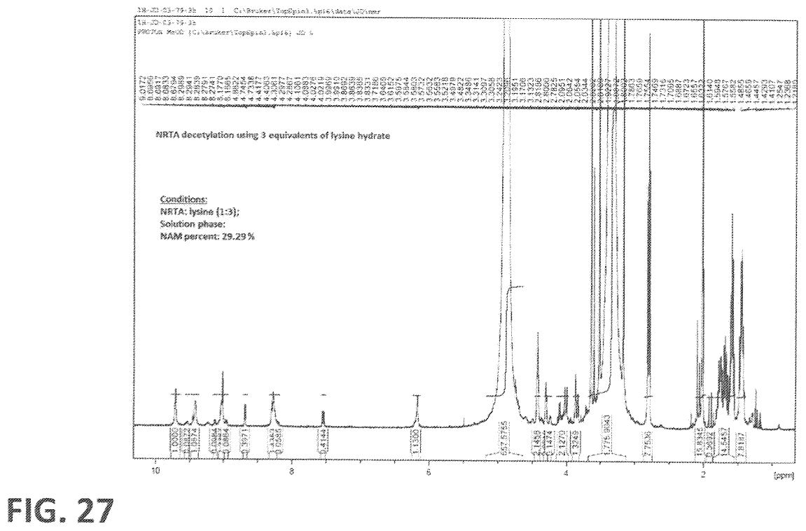

To enhance the rate of the reaction, experiments were conducted using 3 equivalents of lysine in lieu of 1 equivalent. It was found that further increasing the amount of lysine did not affect the rate of the reaction, so 1 or 3 equivalent of lysine was used for the deacetylation experiments. NRTA deacetylation using 3 equivalents of lysine hydrate is shown in FIG. 27.

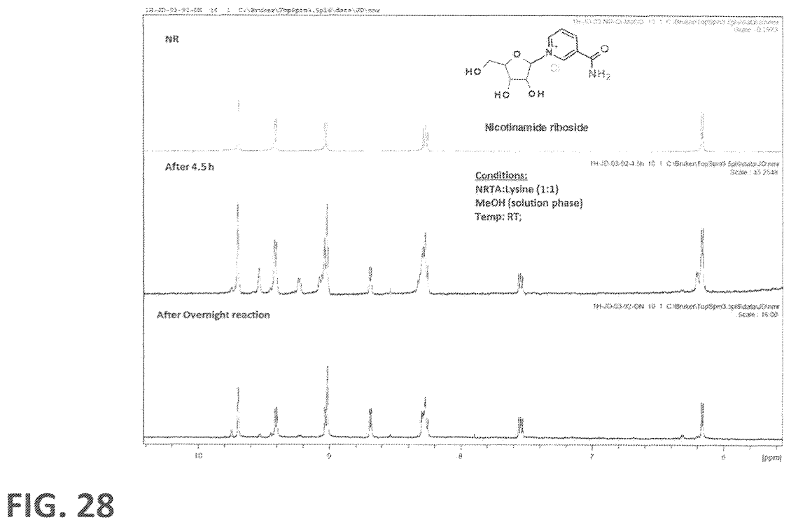

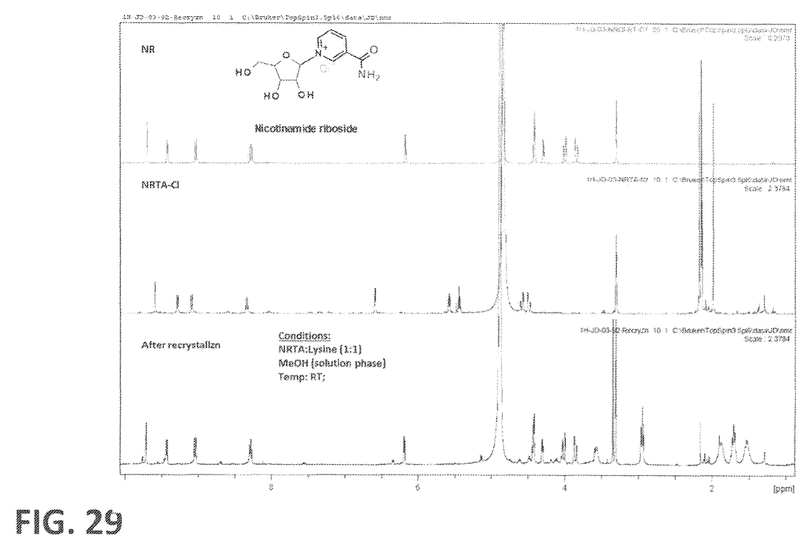

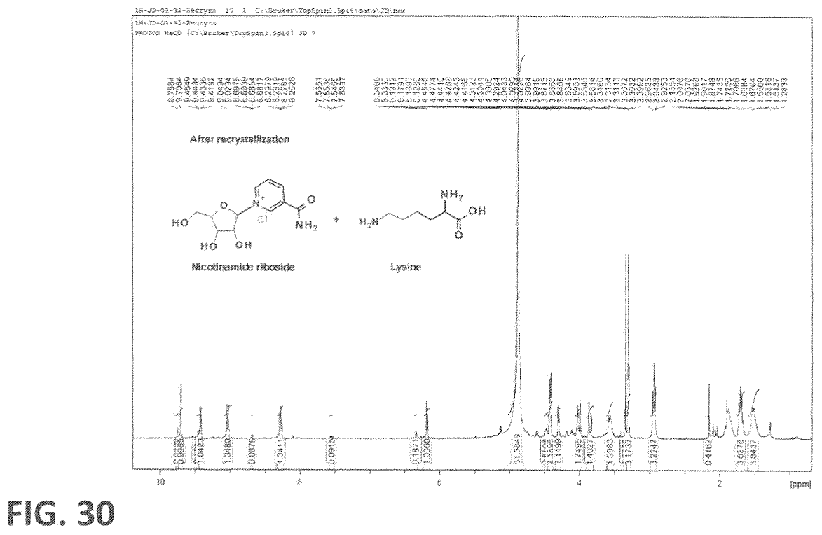

As moisture can potentially be detrimental to the product formation (NR), precautions were taken to minimize or eliminate any traces of moisture by thoroughly drying the reagents and solvents (methanol) before the reaction. During optimization studies for this Example, it was deemed that any trace amount of moisture could result in and/or enhance the degradation of NR to NAM. Considering the solubility of the reaction substrates (NRCl and lysine), methanol was selected as an ideal solvent. Exemplary studies using methanol as a solvent are shown in FIG. 28, FIG. 29, and FIG. 30.

### Optimization of Reaction Conditions for NRHTA Deacetylation

Keeping in mind the reaction conditions required for the deacetylation of NRTA, initial attempts were made to deacetylate NRHTA using 1 equivalent of lysine. However, use of stoichiometric amount of lysine always led to partial deacetylation, and so the ratio of lysine to NRHTA was increased to push the reaction to completion. The studies revealed that a quantity of 3 equivalents of lysine is required for the complete deacetylation of NRHTA. If the reaction were to be conducted at room temperature using 3 equivalents of lysine, then the complete deacetylation would require 3 days (72 hours). The effect of temperature on the rate of reaction was evaluated. In further investigations at elevated temperatures, it was determined that there was a significant difference in the rate of reaction with increased temperature. Results showed that deacetylation could be effectively conducted at 45-50° C. without compromising the quality of the product or resulting in the formation of any undesired side-products as opposed to NR. Methanol was selected as an ideal solvent after considering the solubility of NRHTA and lysine in various solvents.

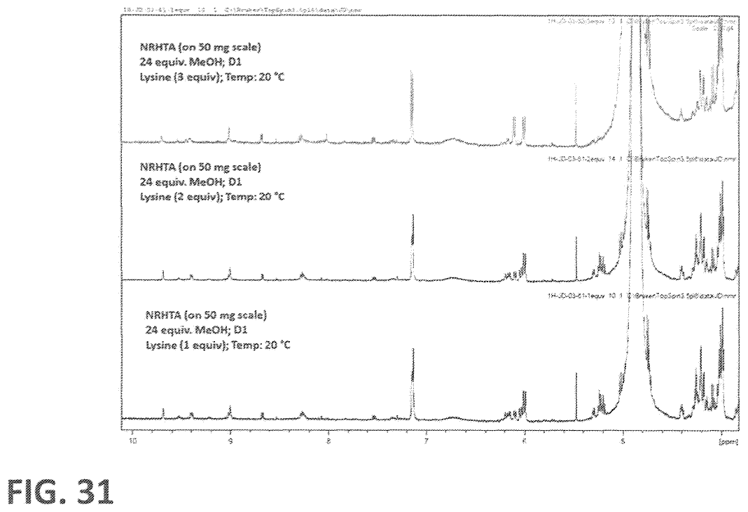

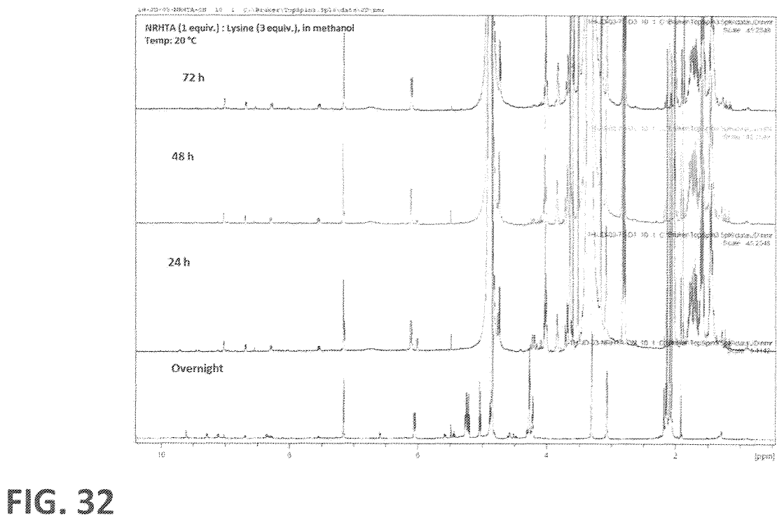

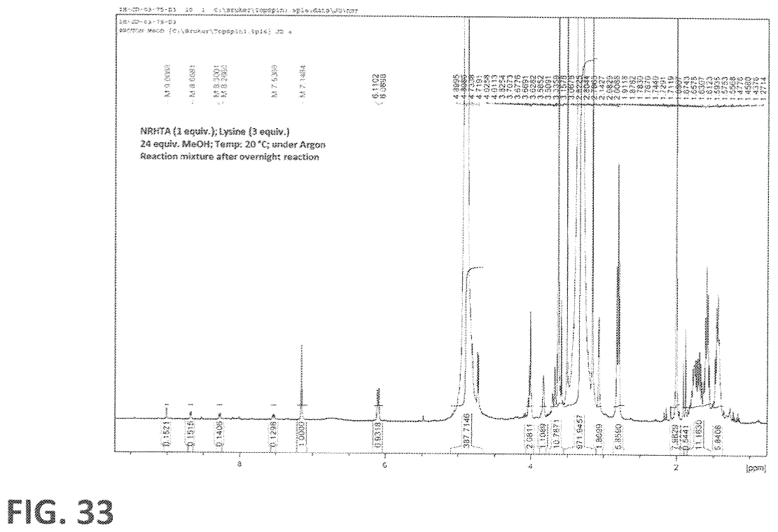

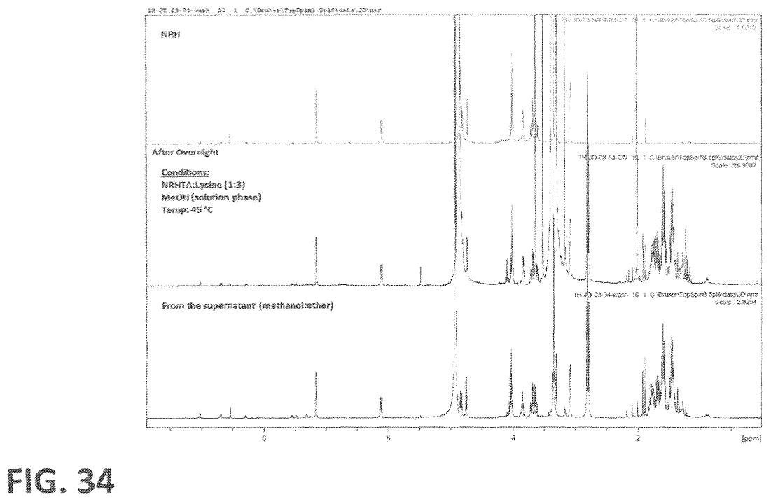

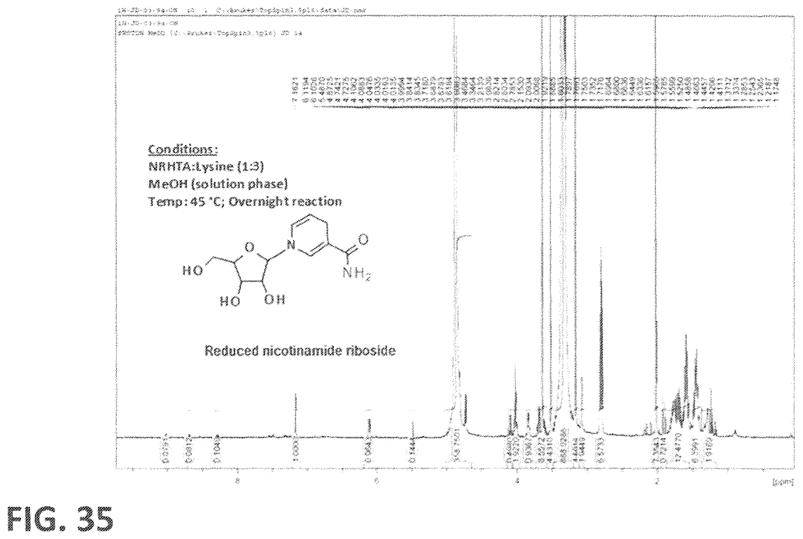

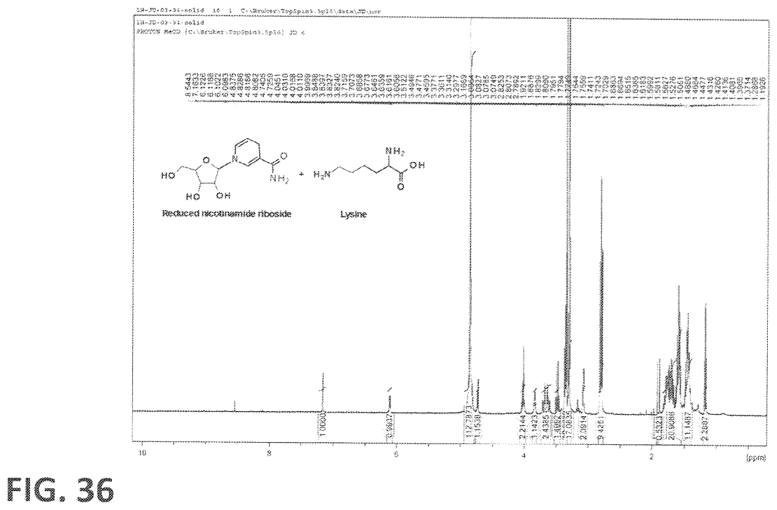

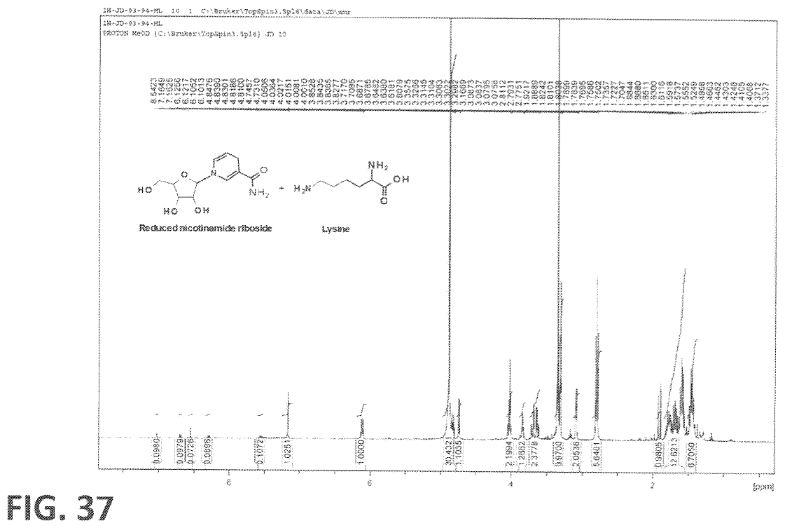

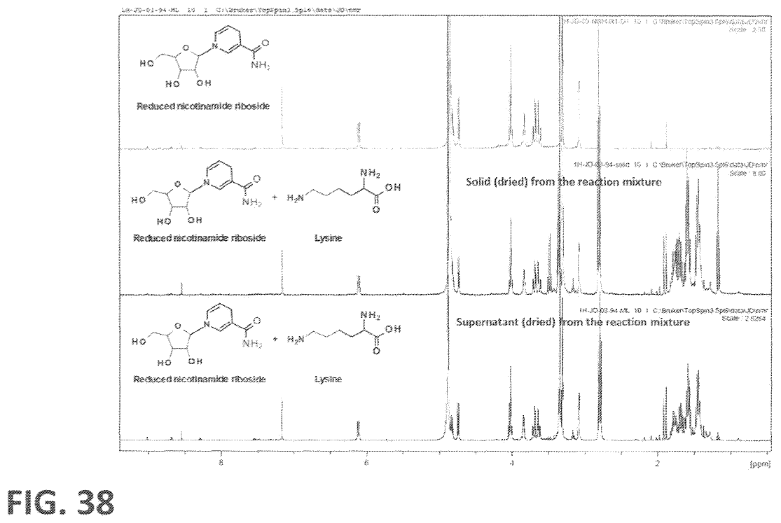

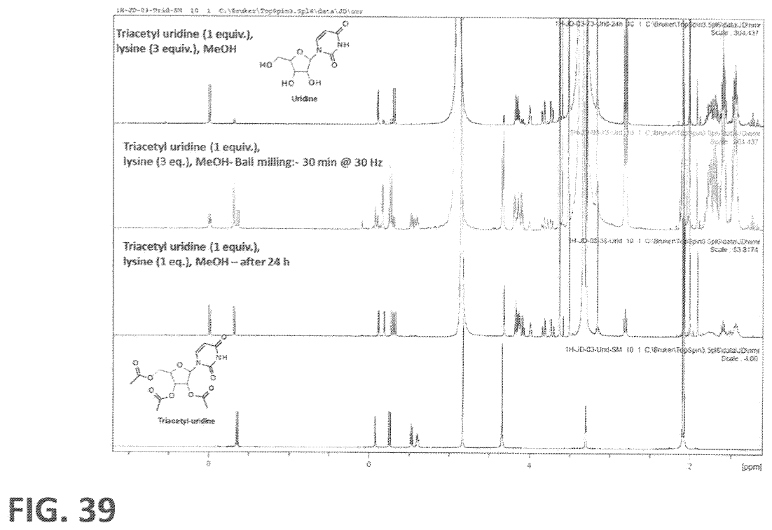

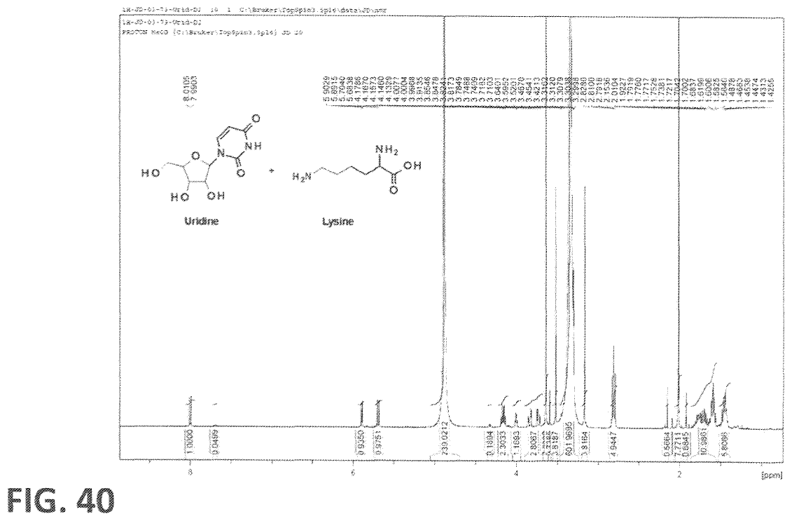

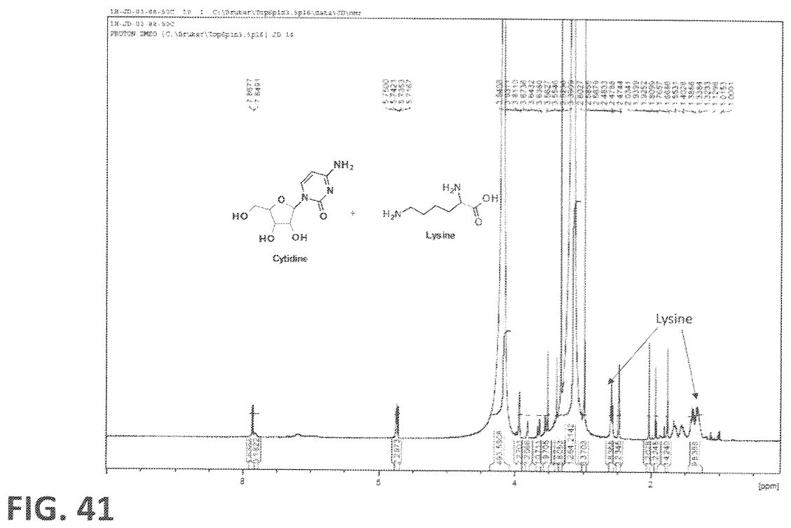

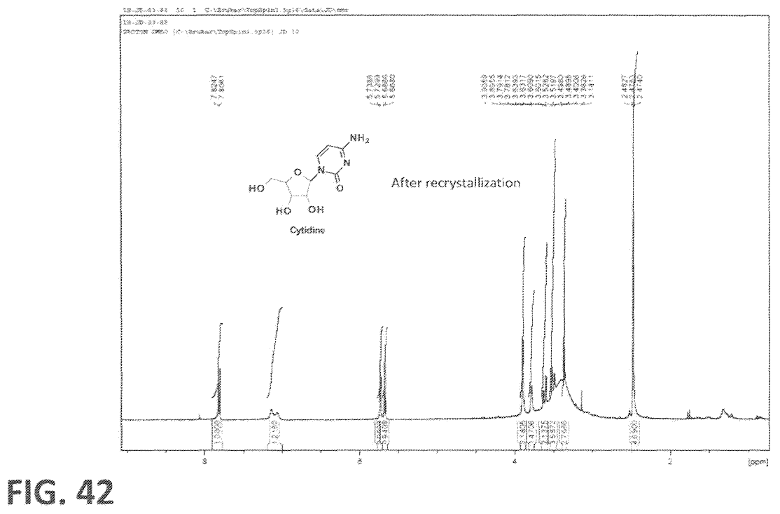

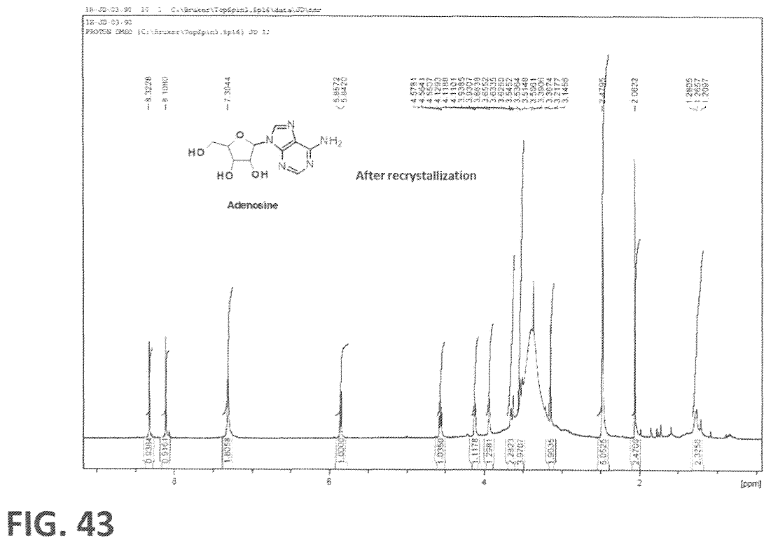

Keeping in consideration of all the above results, it was concluded that optimum conditions for NRHTA deacetylation require approximately 3 equivalents of lysine, a temperature of 45-50° C., and methanol (degassed) as a solvent. With these optimum conditions in hand, NRHTA deacetylation was conducted, and the reaction was successfully completed overnight. Results are shown in FIG. 31, FIG. 32, FIG. 33, FIG. 34, FIG. 35, FIG. 36, FIG. 37, FIG. 38, FIG. 39, FIG. 40, FIG. 41, FIG. 42, and FIG. 43. Compounds and products for the above studies are shown in Table 1.

Insights from the NMR Studies on the Mechanism of Lysine Assisted Deacetylation

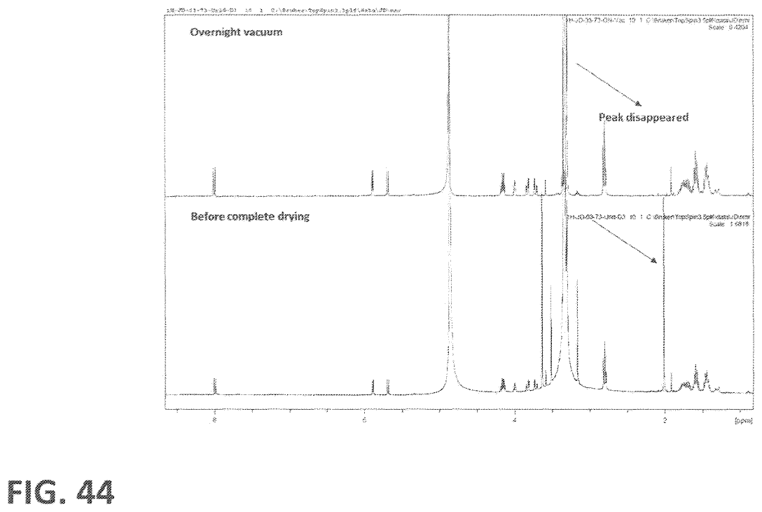

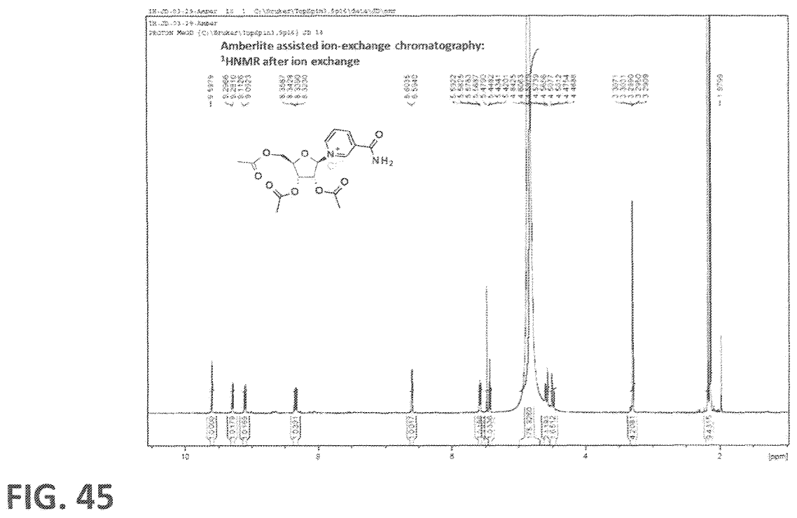

After the deacetylation reaction of 2′, 3′, 5′-O-triacetyl uridine with lysine, two intense peaks were noticed around 2.01 ppm and 3.64 ppm corresponding to an acetyl peak and a methoxy peak. These peaks were not linked to either uridine or lysine and suggested the presence of methyl acetate as an impurity resulting from the nucleophilic attack of methanol on the acetyl group catalyzed by the amino acid acting as a base. This is confirmed from studies known in the art and the from the ¹HNMR which showed the disappearance of peaks exclusively at 2.01 and 3.44 ppm (with other peaks intact) after the samples had been placed overnight under vacuum. This confirmed the formation of the volatile impurity methyl acetate, as shown in FIG. 44. Also, this confirms that lysine is neither consumed in the reaction nor converted to the side-product, α- or/or ε—N—Ac-lysine, but rather acts as a proton shuttle between the substrates. The structural integrity of lysine from the product NMR highlights the significance of the current technique to generate the mixture of desired nucleoside and lysine to be formulated as a potential nutraceutical given the nutritional benefits of lysine.

### III. Working Example III

### Procedure for the Deprotection of NRTA and NRHTA

The procedure for the deprotection/deacetylation of NRTA to NR by lysine is as follows: A clean, dry round bottom flask was degassed and charged with nicotinamide riboside triacetate (1 equiv.) followed by dry methanol and lysine (1 equiv.) at room temperature and the resulting mixture was stirred for 4-5 h until the disappearance of starting material. Reaction progress was monitored by ¹HNMR. After the reaction was complete, reaction mixture was evaporated to remove methanol and washed with ether, and dried to afford a mixture of NR and lysine.

The procedure for the deprotection of triacetate-NRH (NRHTA) by amino acids is as follows: A clean, dry round bottom flask was flushed with argon (at least 2 times), followed by the addition of reduced nicotinamide riboside triacetate (NRHTA, 1 equiv.) followed by the addition of dried and degassed methanol and lysine (3 equiv.) under argon. The resulting mixture was stirred under argon at 45-50° C. overnight, and reaction progress was monitored by ¹HNMR. After the reaction was complete, supernatant was separated from the solid. Both the solid and supernatant were washed with degassed ether to provide a mixture of NRH and lysine.

The procedure for the deprotection of nucleosides by lysine and methanol is as follows: A clean, dry round bottom flask was flushed with argon (at least 2 times), followed by the addition of the corresponding nucleoside (1 equiv.), lysine (3 equiv.) and anhydrous methanol. The resulting mixture was heated to 50° C. and stirred at the same temperature until the reaction completion. After the reaction was complete, the desired product was collected by recrystallization, dried, and analyzed by NMR.

### IV. Working Example IV

### Reaction

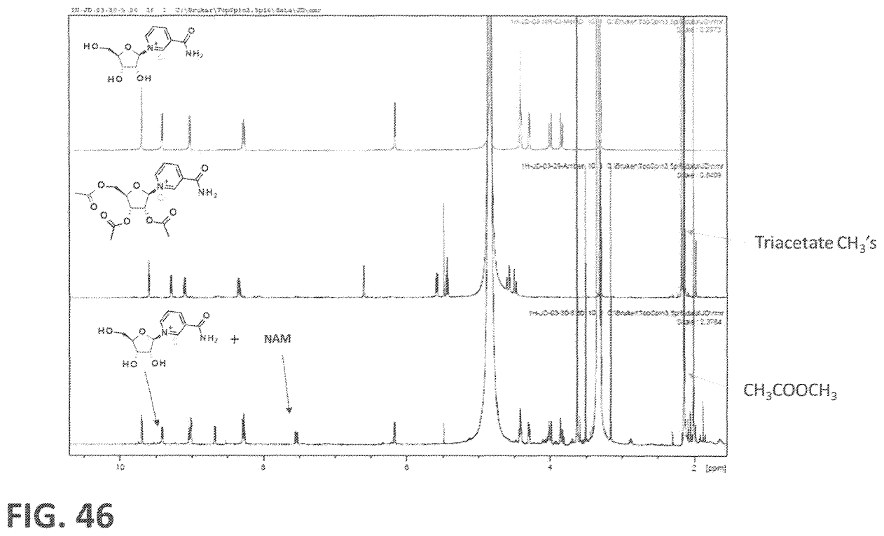

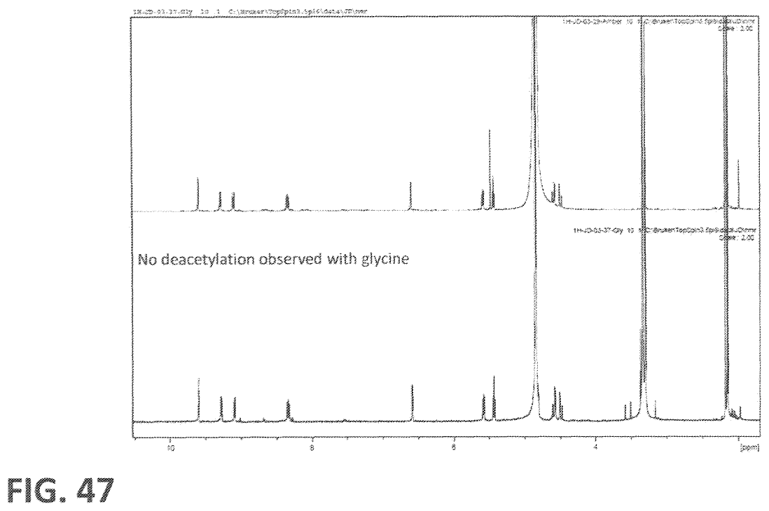

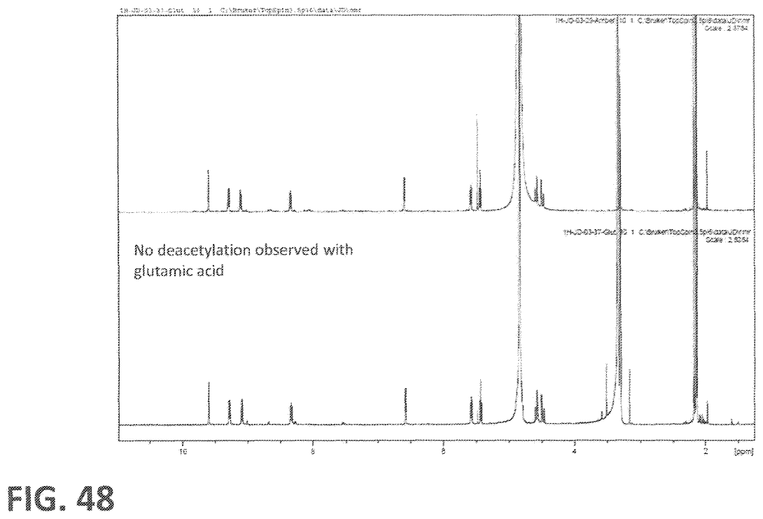

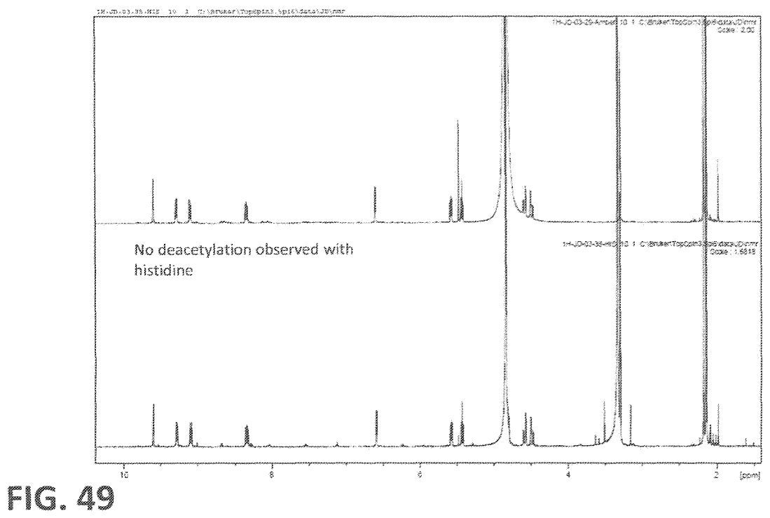

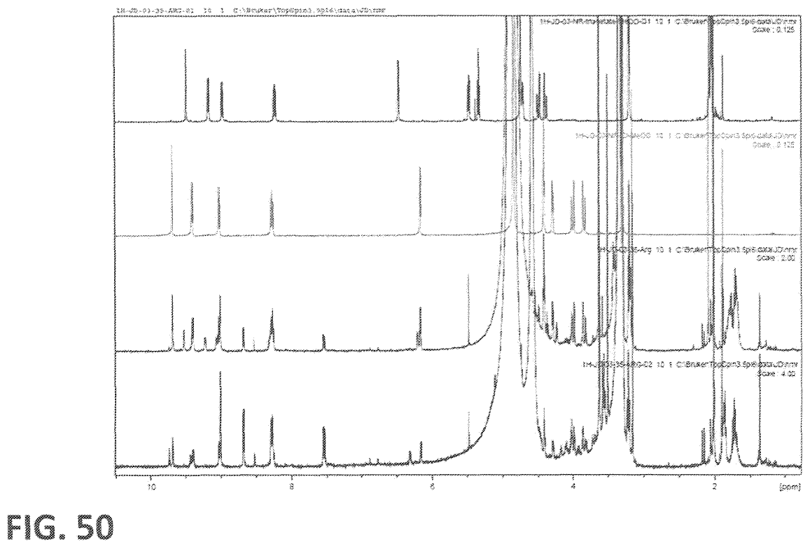

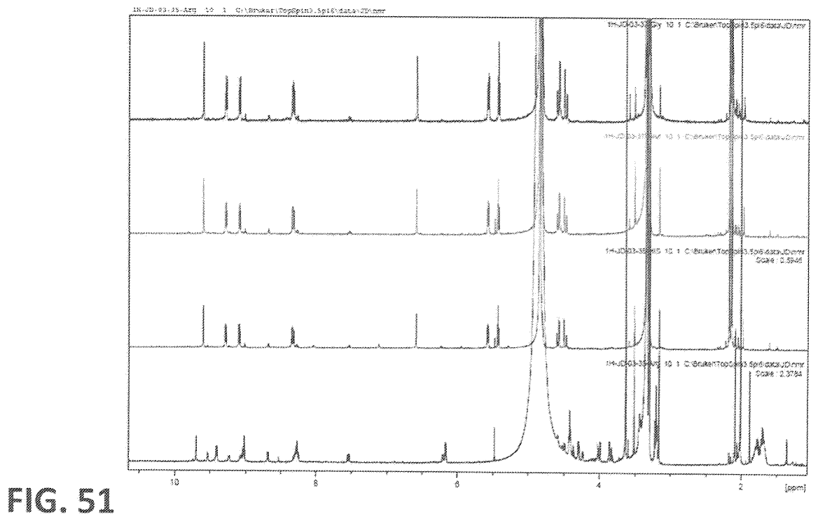

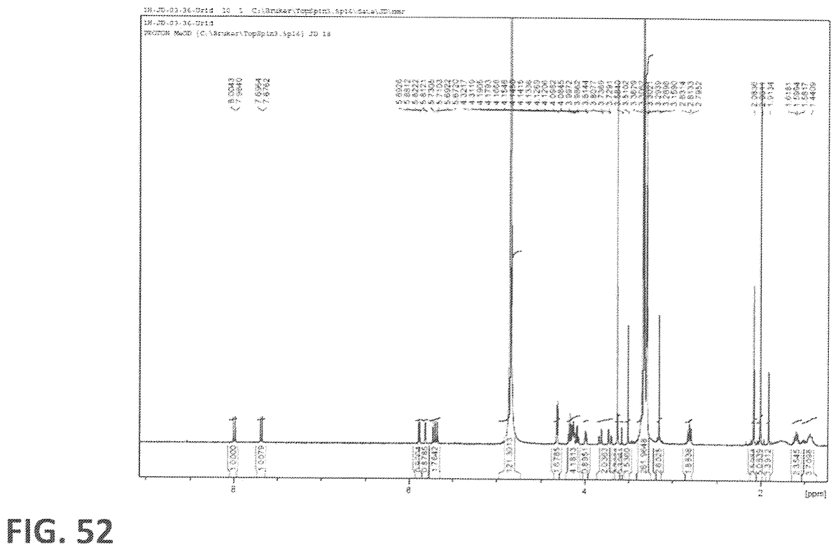

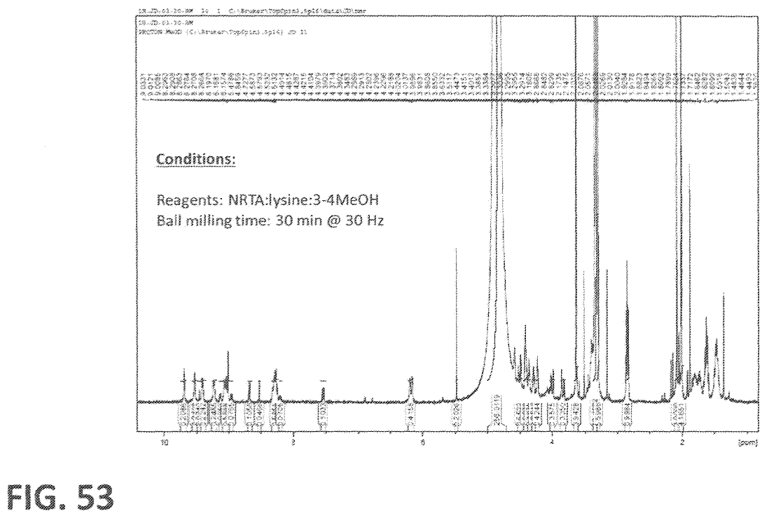

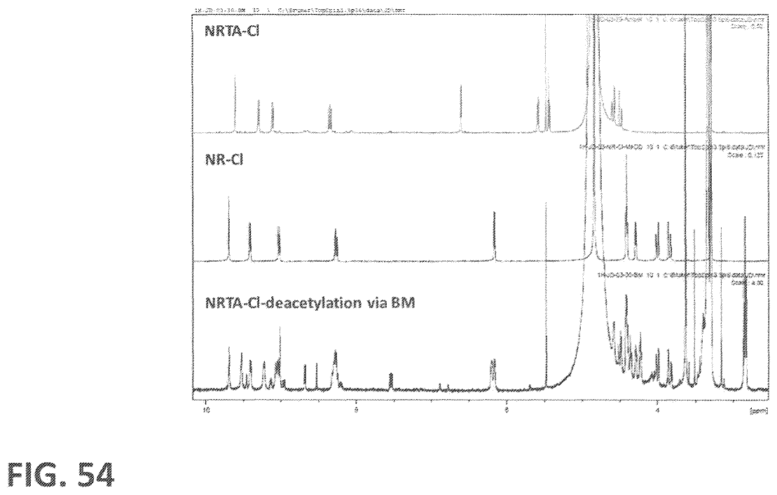

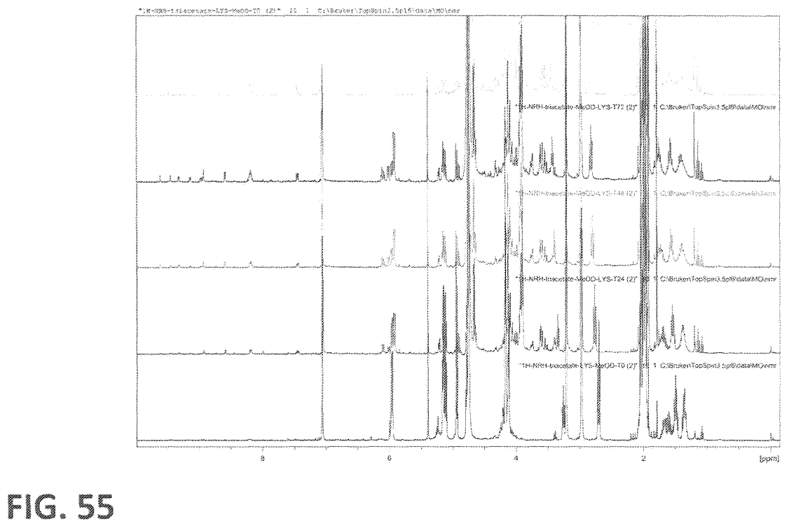

The reaction involves deprotection of NR triacetate to NR by amino acids in methanol at room temperature, in solution and by milling. Results are shown in FIG. 45, FIG. 46, FIG. 47, FIG. 48, FIG. 49, FIG. 50, FIG. 51, FIG. 52, FIG. 53, and FIG. 54. The reaction involves deprotection of NRH triacetate to NRH by amino acids in methanol at room temperature, in solution and by milling. Results are shown in FIG. 55.

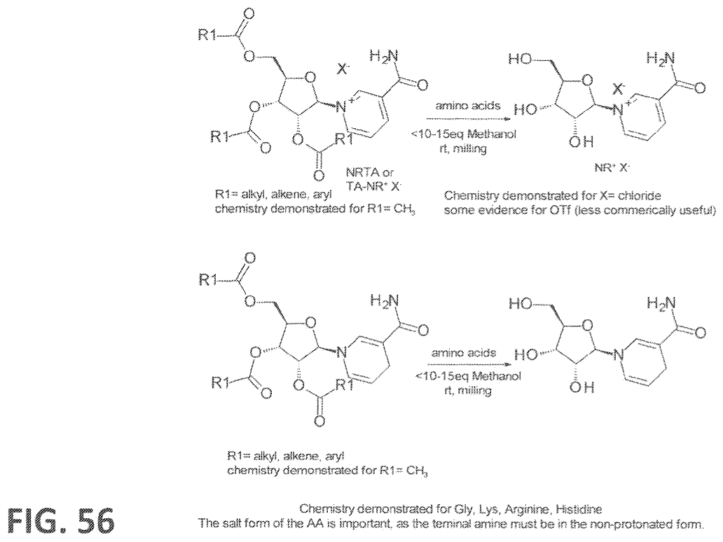

NR-triacetate (triflate or chloride) and reduced NR (NRH) triacetate can be deprotected in methanol (<10-15 equivalents) in presence of basic amino acids (shown in FIG. 56), by milling. The rates of NR hydrolysis to NAM (unwanted product) is less than that of methanolic ammonia conditions currently used in the manufacturing of NR—Cl and can be conducted at room temperature, unlike the ammonia conditions. Crucially, these compete well with the K₂CO₃/methanolic conditions used in BM for the deprotection of NRHTA to NRH. Crucially, lysine is the most efficient, cleanest catalyst (1 molar equivalent) (arginine is close 2nd) and conditions are being improved upon. The by-product of the reaction is a volatile ester, methyl acetate (3 equivalents formed during the process); this triester could be potentially trapped and re-used in other processes. The excess methanol is removed under reduced pressure. The chemistry whereby methanol (3-5 equivalents) is applied with lysine under ball-milling conditions are being optimized. The water content in the NRCl triacetate composition remains the limiting factor in the mole-to-mole conversion of the triacetate to NRCl.

### V. Example V

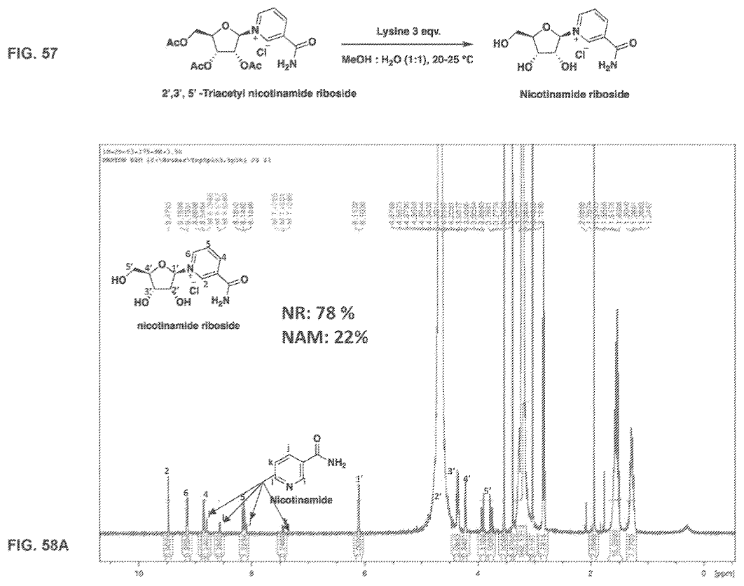

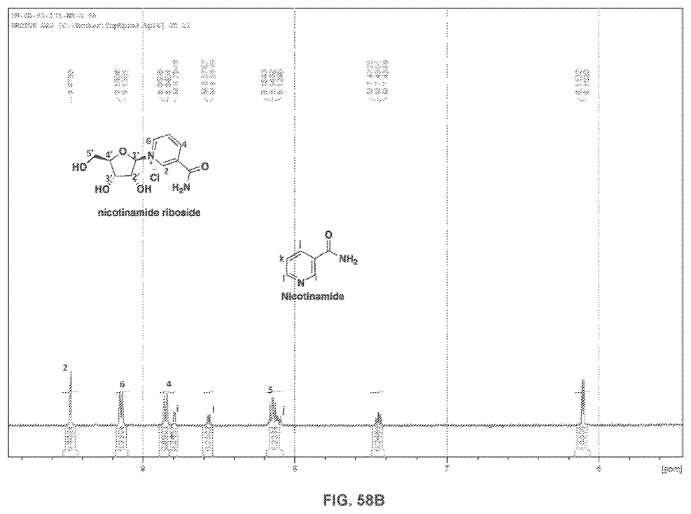

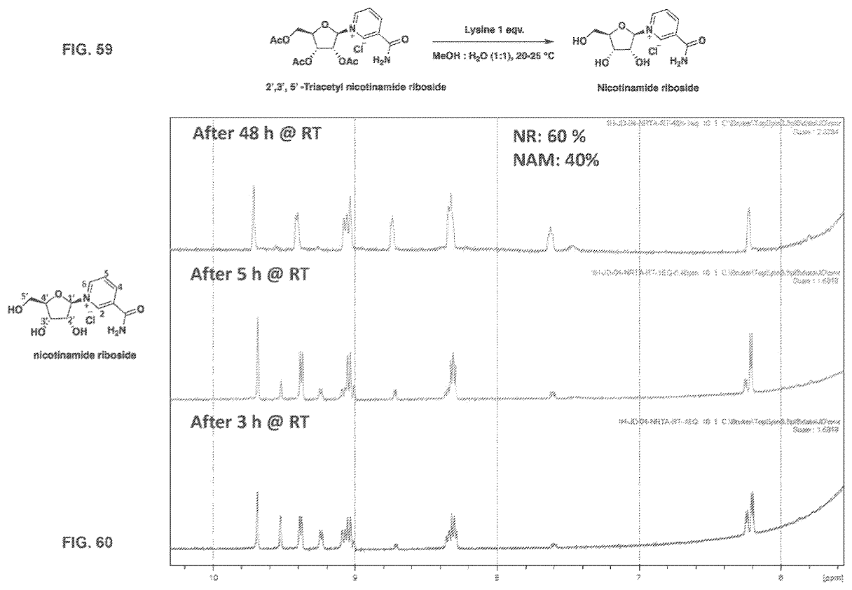

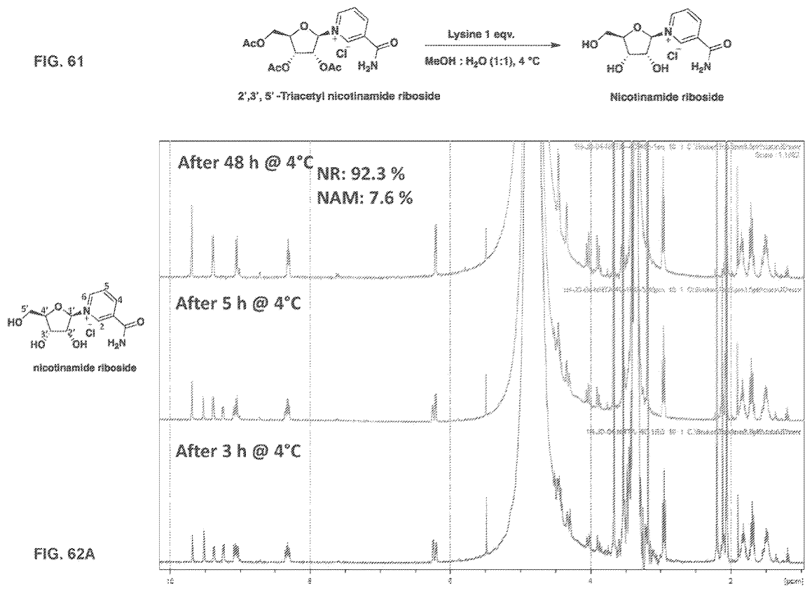

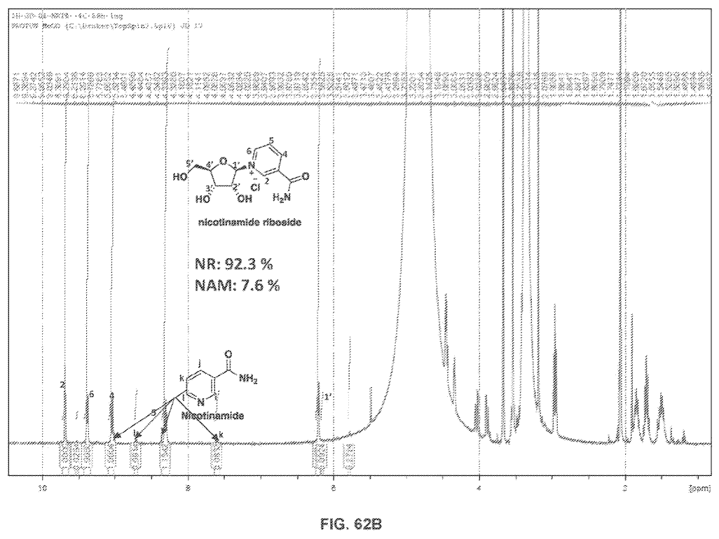

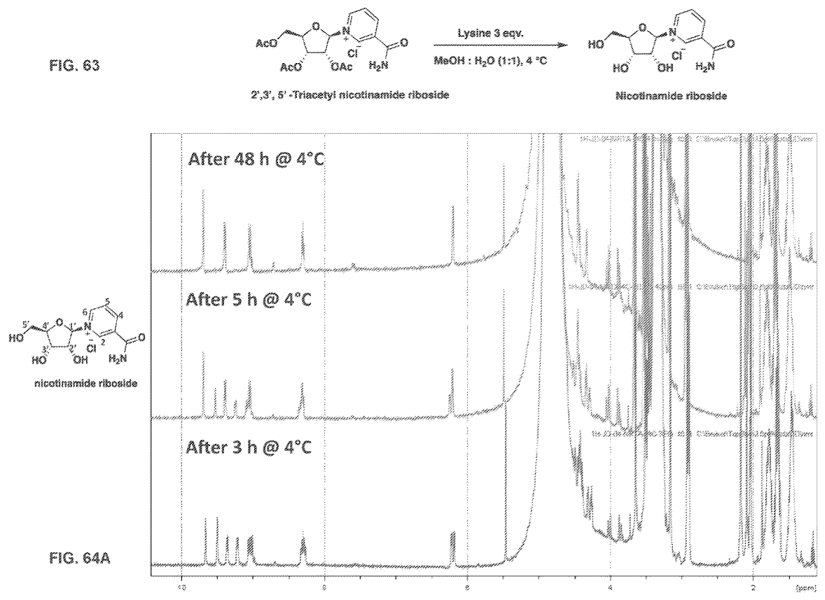

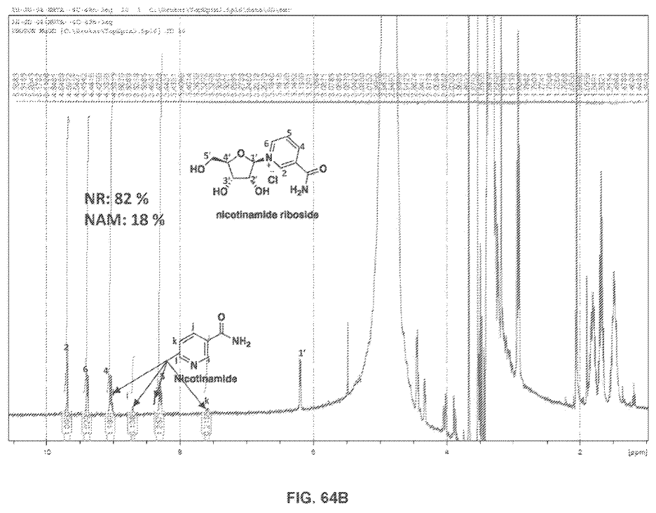

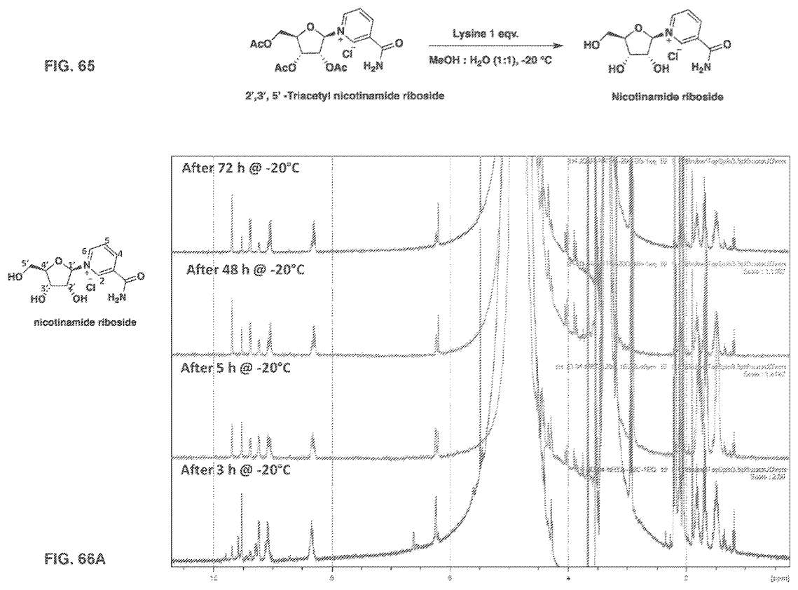

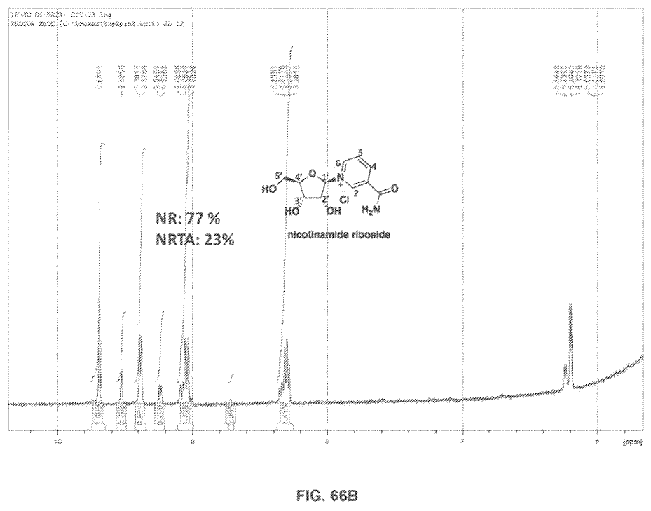

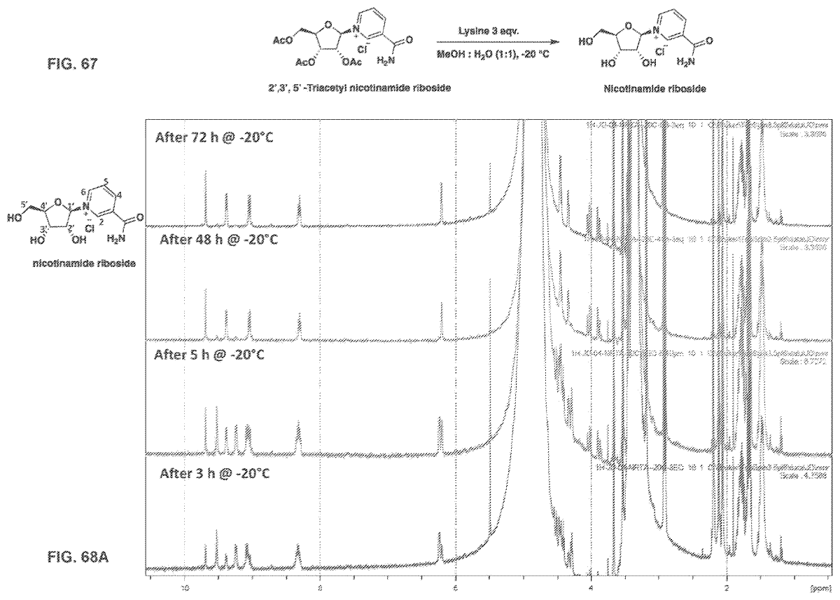

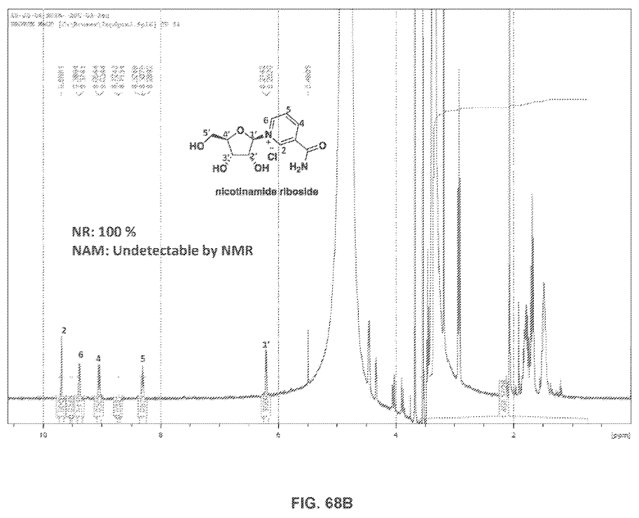

Deacylation of NRTA was tested in a 1:1 mixture of water and MeOH with 1 or 3 equivalents of lysine and at room temperature (see FIGS. 57-60), 4° C. (see FIGS. 61-64), or −20° C. (see FIGS. 65-68). NR deacylation was observed with all reaction conditions, with 72 hours at −20° C. and 3 equivalents of lysine yielding 100% conversion of NR with no NAM degradation. A summary of the results is provided in Table 1.

TABLE 1
NRTA Deacylation in 1:1 Water:MeOH

| Time | Temperature | Lysine (eq.) | NR | NAM | NRTA (SM) |
|---|---|---|---|---|---|
| 3.5 h | Room temp | 1 | 78% | 22% | 0 |
| 48 h | Room temp | 3 | 60% | 40% | 0 |
| 48 h | 4° C. | 1 | 92.3% | 7.6% | 0 |
| 48 h | 4° C. | 3 | 82% | 18% | 0 |
| 72 h | −20° C. | 1 | 77% | 0 | 23% |
| 72 h | −20° C. | 3 | 100% | 0 | 0 |

### VI. Example VI

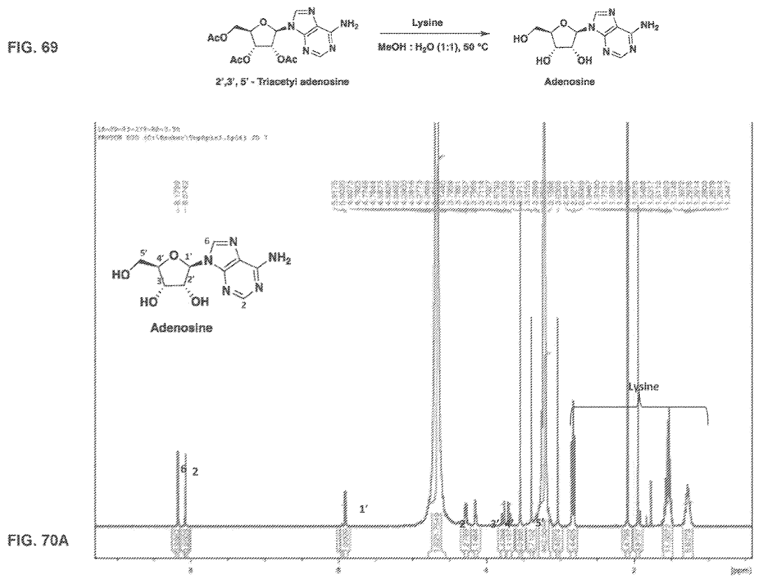

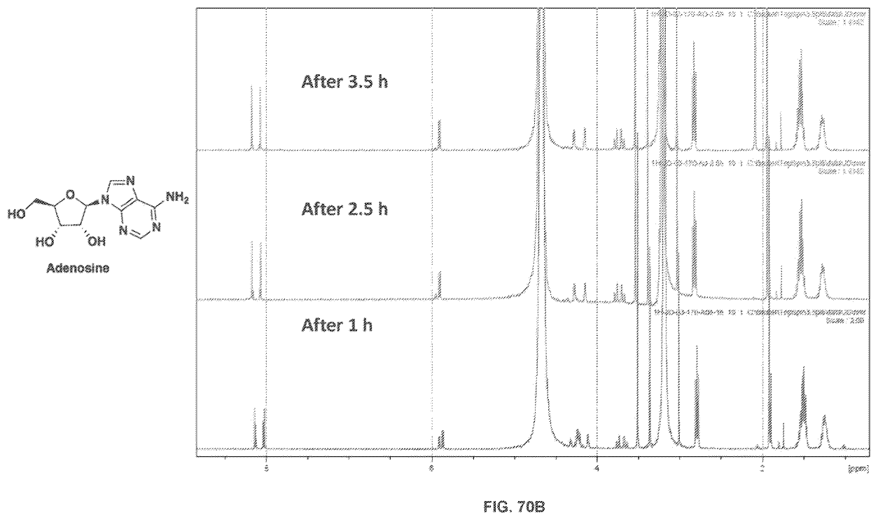

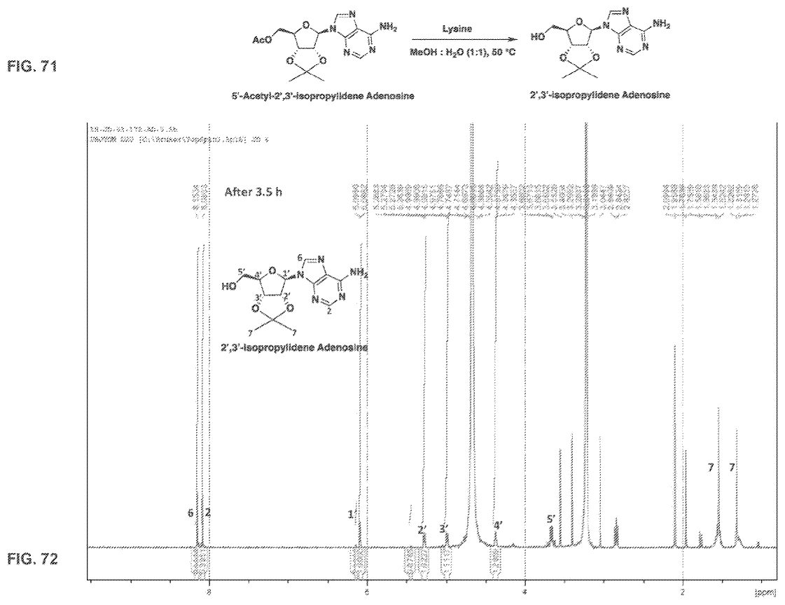

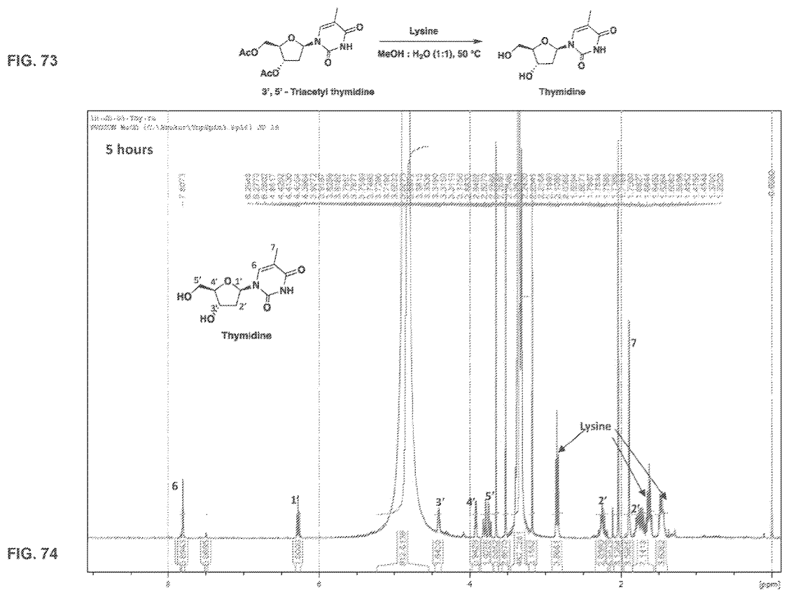

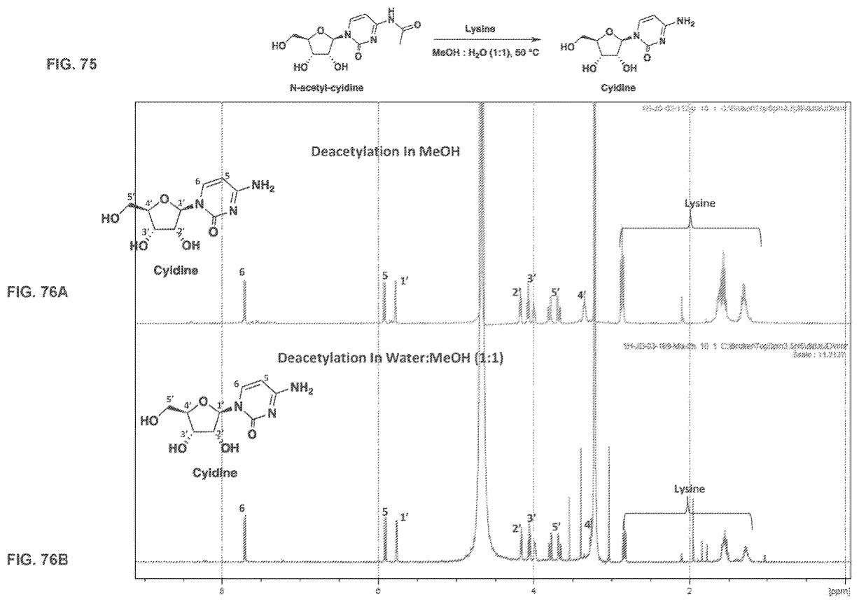

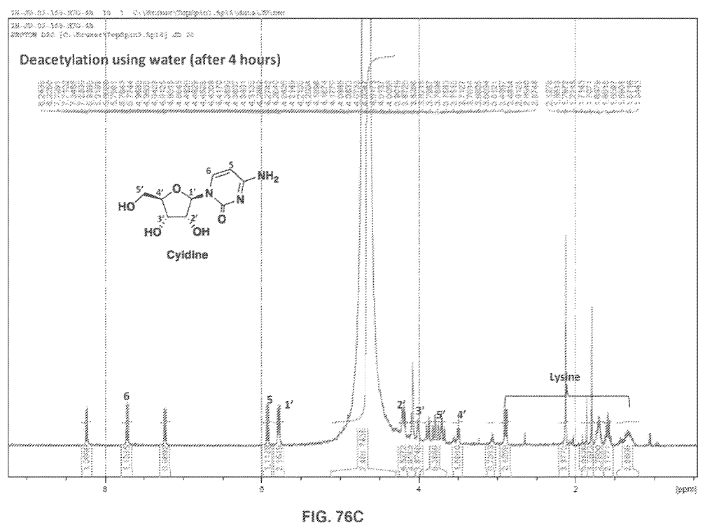

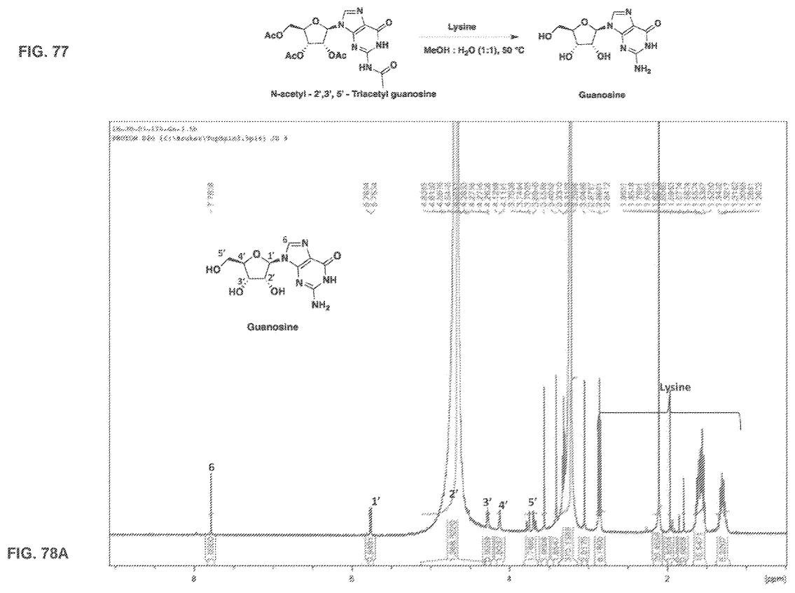

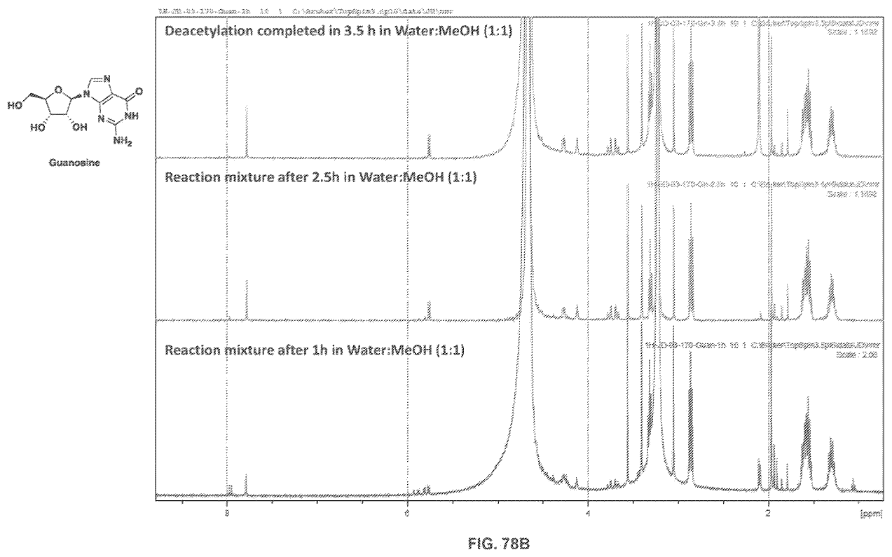

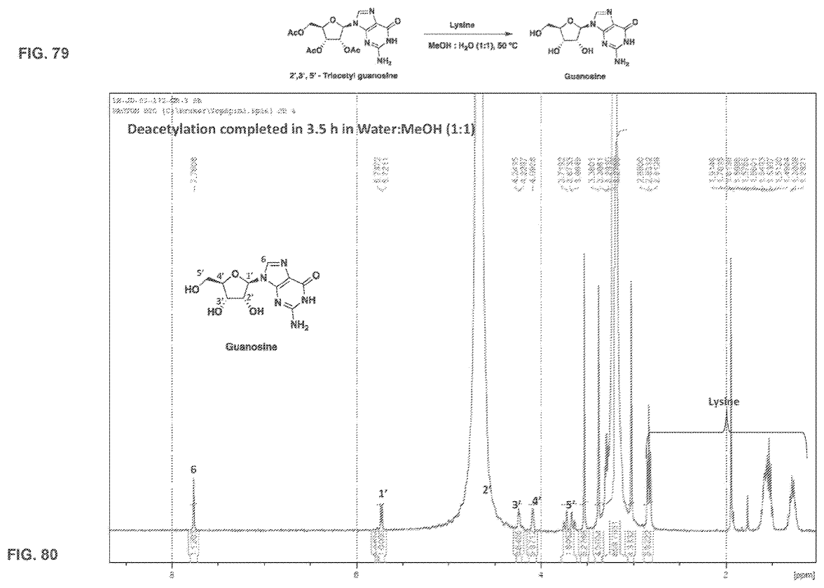

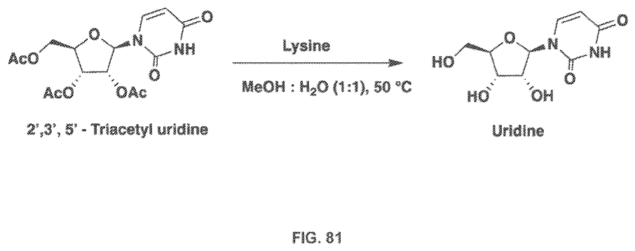

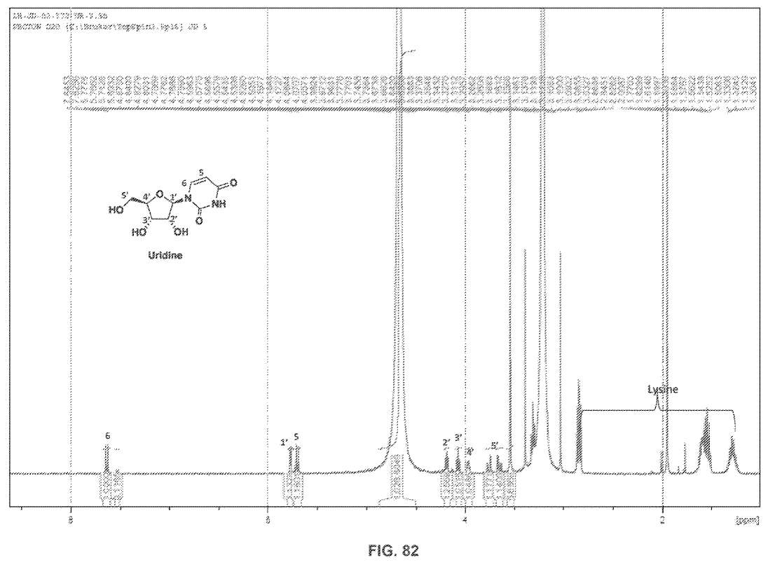

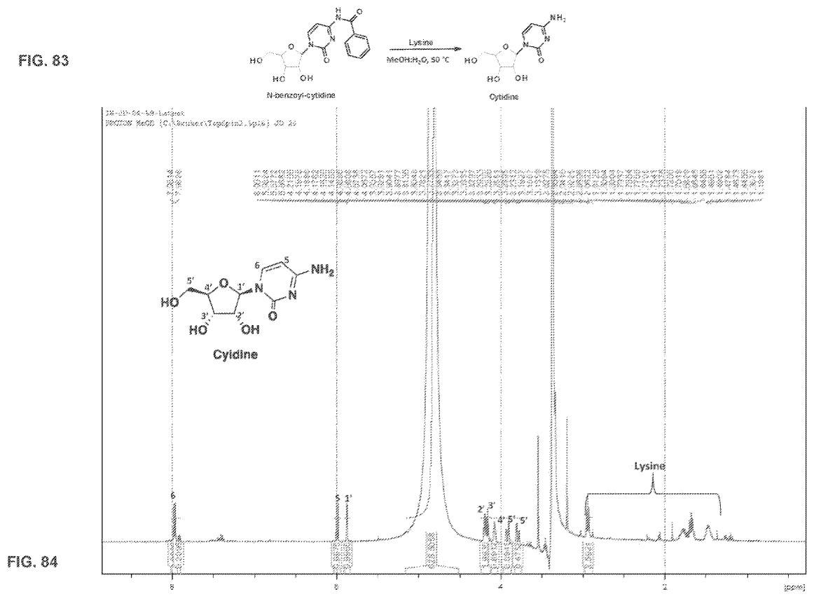

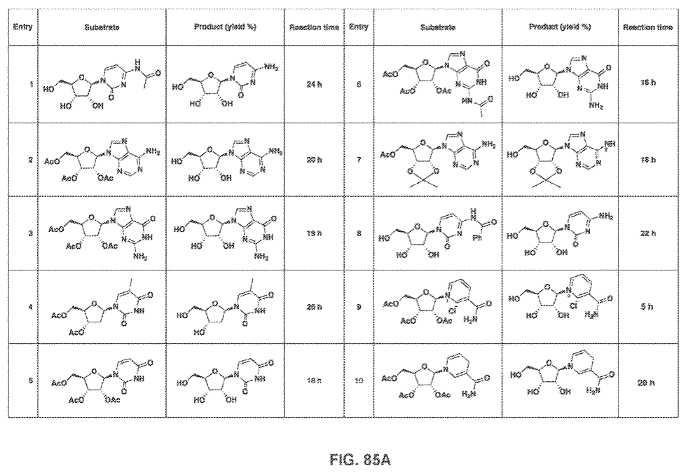

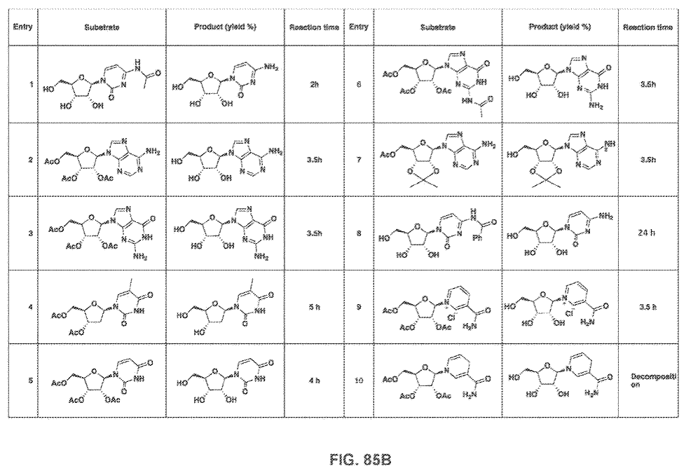

Deacylation of various acylated ribosides was tested in a 1:1 mixture of water and methanol (MeOH), as shown in FIGS. 69-70B show the conversion of 2′,3′, 5′-triacetyl adenosine to adenosine in 3.5 hours. FIGS. 71-72 show the conversion of 5′acetyl-2′,3′-isopropylidene adenosine to 2′,3′-isopropylidene adenosine in 3.5 hours. FIGS. 73-74 show the conversion of 3′,5′-diacetyl thymidine to thymidine in 5 hours. FIGS. 75-76C shows the conversion of N-acetyl-cytidine to cytidine. FIGS. 76A, 76B, and 76C show the reaction as performed in MeOH alone, a 1:1 water:MeOH mixture, and water alone, respectively. FIGS. 77-78B show the conversion of N-acetyl-2′,3′,5′-triacetyl guanosine to guanosine in 3 hours. FIGS. 79-80 show the conversion of 2′,3′,5′-triacetyl guanosine to guanosine in 3.5 hours. FIGS. 81-82 show the conversion of 2′,3′,5′-triacetyl uridine to uridine in 4 hours. FIGS. 83-84 show the conversion of N-benzoyl-cytidine to cytidine in 24 hours.

FIGS. 85A and 85B show the required conversion time of various acylated ribosides into their corresponding deacylated ribosides in MeOH (FIG. 85A) or in a 1:1 mixture of water:MeOH (FIG. 85B). The 1:1 water:MeOH mixture afforded significantly faster reaction rates.

### VII. Example VII

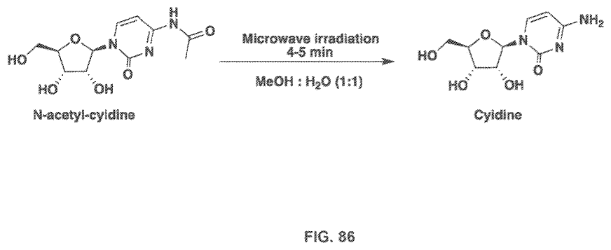

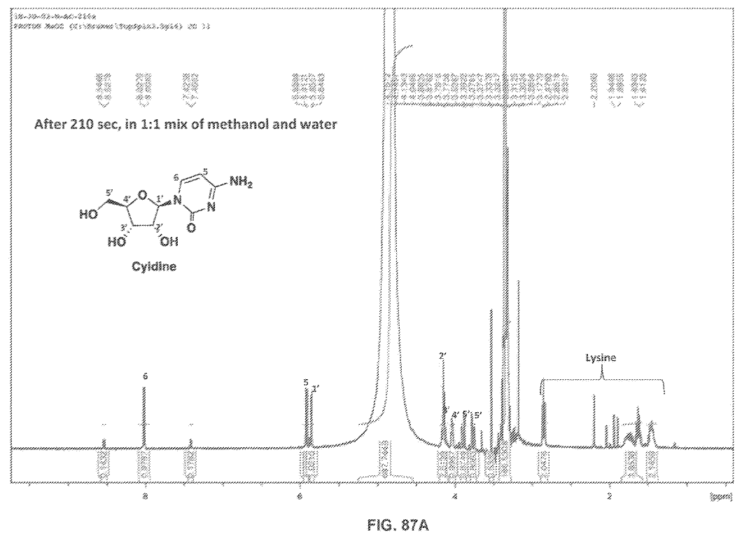

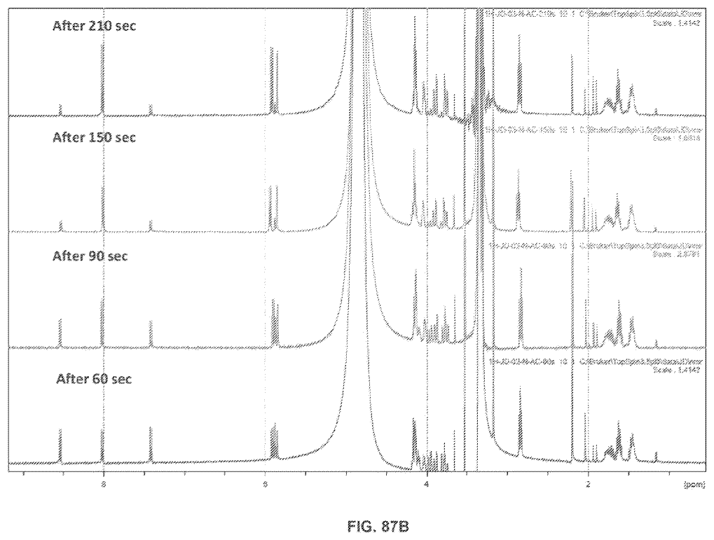

Microwave irradiation was utilized for the conversion of N-acetyl cytidine to cytidine (FIG. 86). A mixture of N-acetyl cytidine (52 mg, 0.17 mmoles), lysine (25 mg, 0.17 mmoles) in a mixture of methanol and water (1:1; 0.5 mL each) was subjected to microwave irradiation, and NMR was recorded for every 30-60 sec of microwave irradiation (FIG. 87B) Reaction mixture showed gradual deacetylation to afford the desired product of cytidine. At the end of 210 sec, reaction mixture showed 83% of cytidine (FIG. 87A).

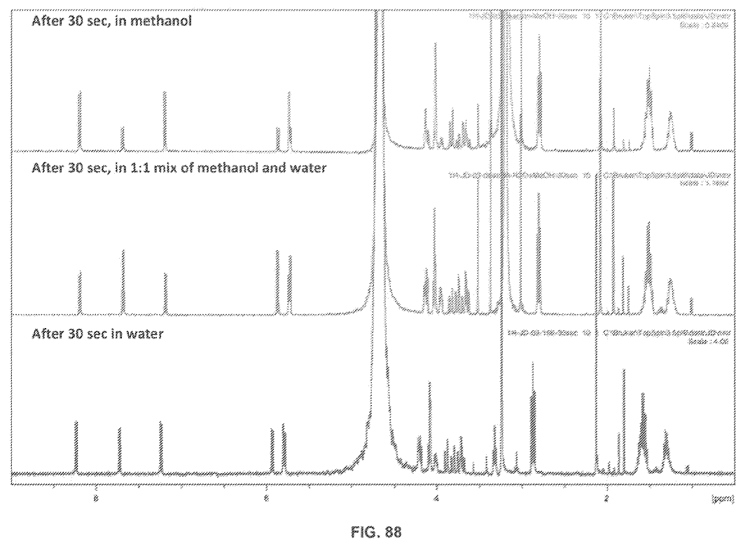

The microwave reaction was also performed in water and methanol separately, and the results at 30 seconds are shown in FIG. 88. The 1:1 water:MeOH mixture afforded the fastest reaction rate.

### VIII. Example VIII

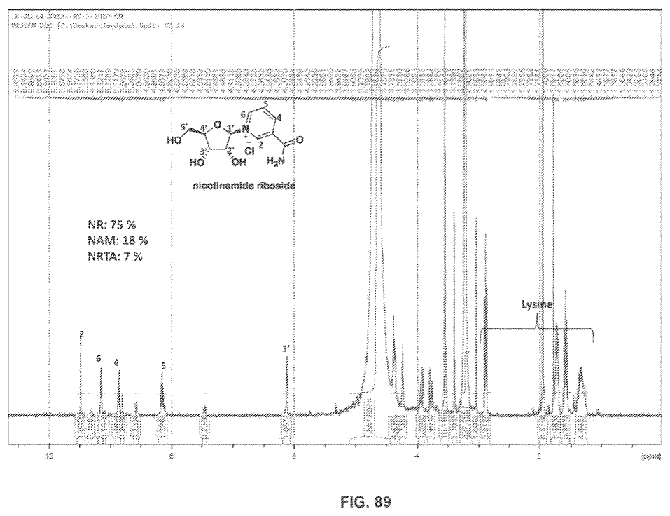

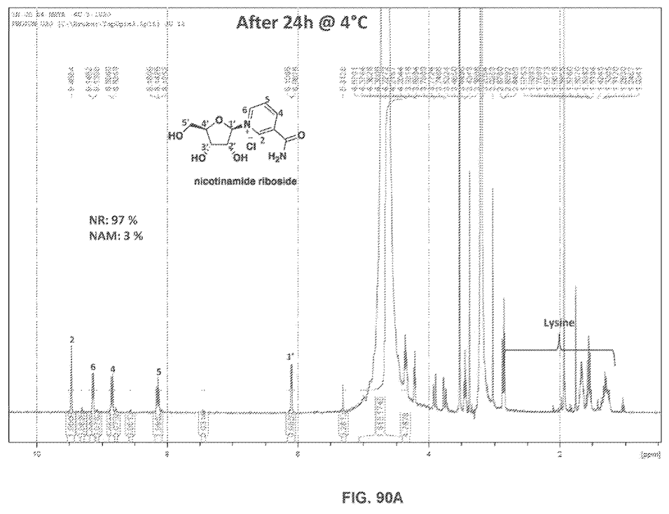

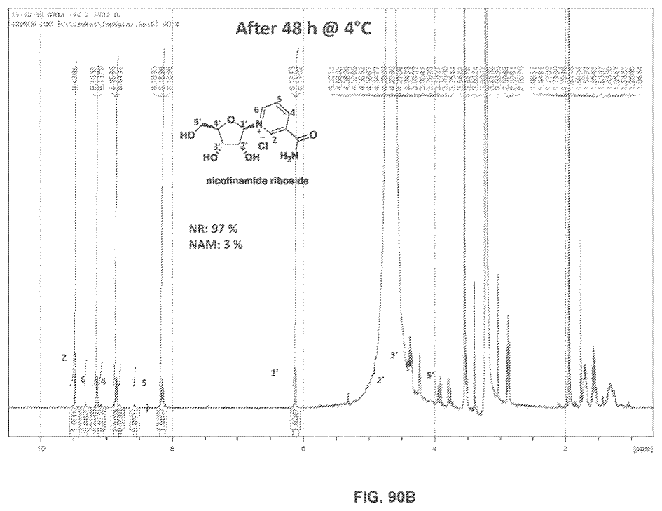

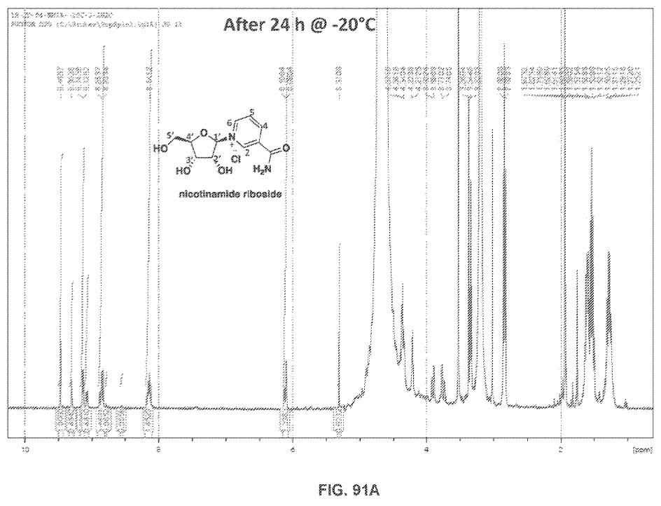

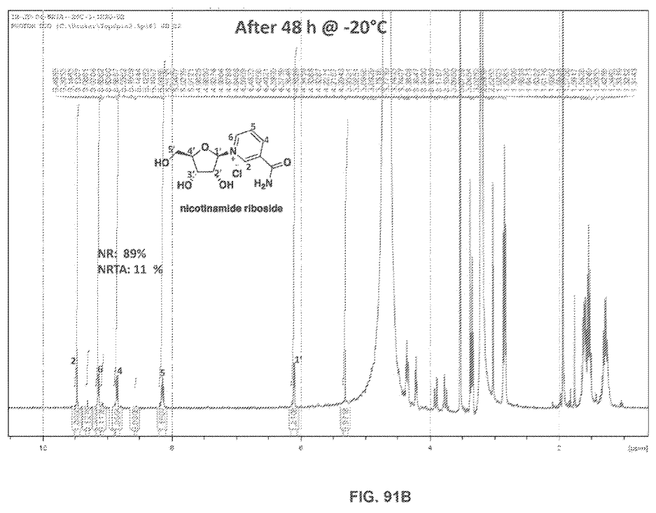

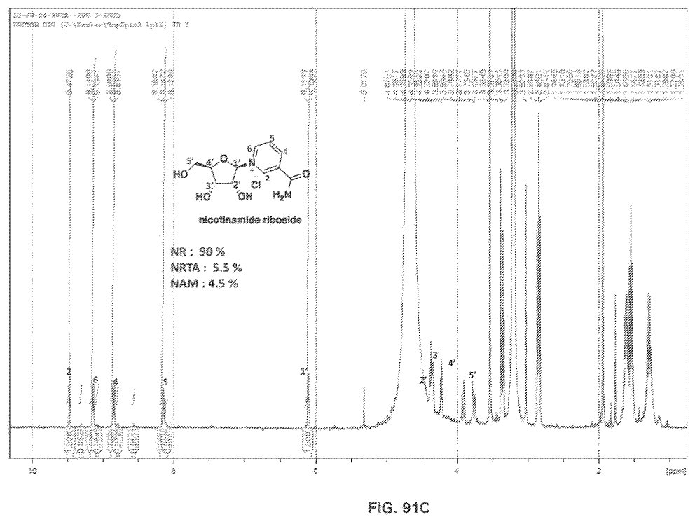

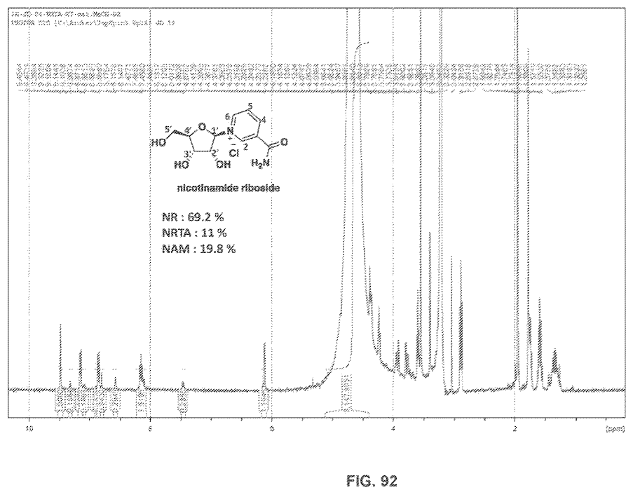

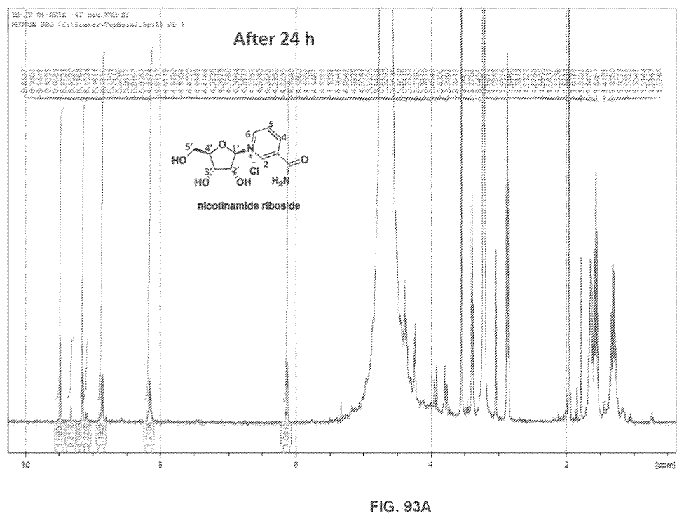

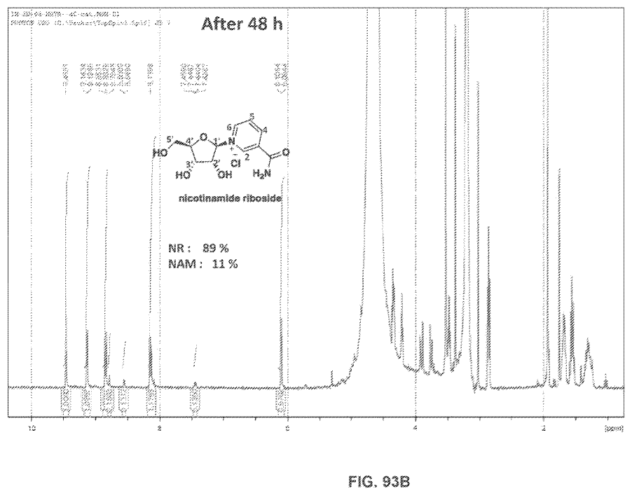

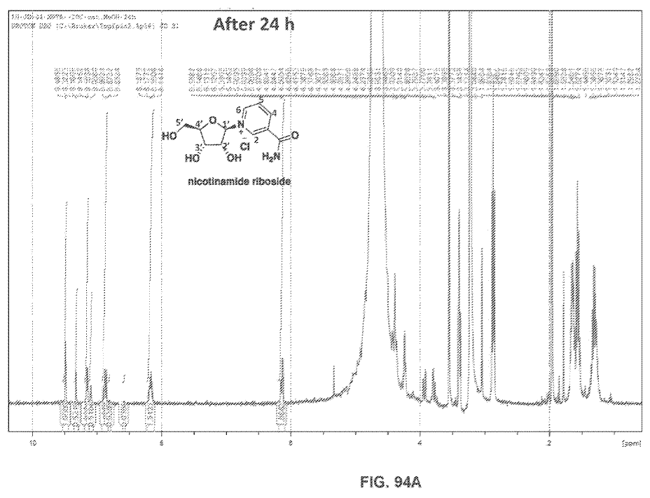

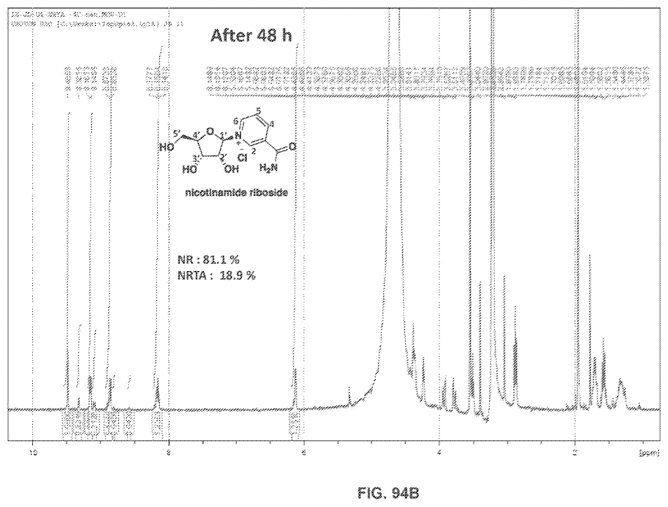

Different ratios of water and MeOH, temperatures, and amount of lysine were tested for the conversion of NRTA to NR (FIGS. 89-94B). NR deacylation was observed in all reaction conditions. Of the tested conditions, a 3:1 water:MeOH ratio at −20° C., 1 equivalent of lysine, and 4° C. had the highest yield and no degradation products. Results are summarized in Table 2.

TABLE 2
NRTA Deacylation in Different Ratios of Water:MeOH

| H₂O:MeOH (ratio) | Time | Temperature | | Lysine (eq.) | NR | NAM | NRTA (SM) |
|---|---|---|---|---|---|---|---|
| 3:1 | 16 h | 20-25° | C. | 1 | 75.1% | 17.3% | 7.5% |
| 3:1 | 24 h | 4° | C. | 1 | 97% | 3% | 0 |
| 3:1 | 48 h | −20° | C. | 3 | 89% | 0 | 11% |
| 3:1 | 48 h | −20° | C. | 3 | 90% | 4.5 | 5.5% |
| 24:1 | 48 h | 20-25° | C. | 1 | 69.2% | 19.8% | 11% |
| 24:1 | 48 h | 4° | C. | 1 | 89% | 11% | 0 |
| 24:1 | 48 h | −20° | C. | 3 | 81.1% | 0 | 18.9% |

### IX. Example IX

Purification of nucleoside-lysine mixtures is performed as follows. Nucleoside-lysine mixtures, including NRCl-lysine mixtures, is purified by precipitation, recrystallization, and/or chromatography techniques. Chromatography techniques include reversed-phase column liquid chromatography and medium and high pressure liquid chromatography. Alternatively, beads or columns that adsorb lysine is applied to separate lysine from nucleosides, and is applied to NRCl-lysine mixtures (see, e.g., Finkler et al., Journal of Biotechnology 324S (2020):100024; https://doi.org/10.1016/j.btecx.2020.100024).

### X. Example X

Poly-lysine, i.e., lysine immobilized on a polymer or polymeric form of lysine as described herein, is tested as a reactant for the deacetylation of nucleosides, using water/methanol as co-reagents, to deprotect nucleosides. The poly-lysine is the immobilized L-lysine from G-Biosciences (catalog number 786-1371) and/or the poly-L-lysine solution from Sigma-Aldrich (P8920). Since poly-lysine is a large polymer, it can be filtered after the reaction is completed to simplify the product purification process. Immobilized lysine renders lysine-free NRCl by filtration of the reaction mixture and removal of excess methanol and water, including the volatile methyl ester. The filtered poly-lysine can be recycled similarly to that of immobilized morpholine, as described in Example XI.

### XI. Example XI

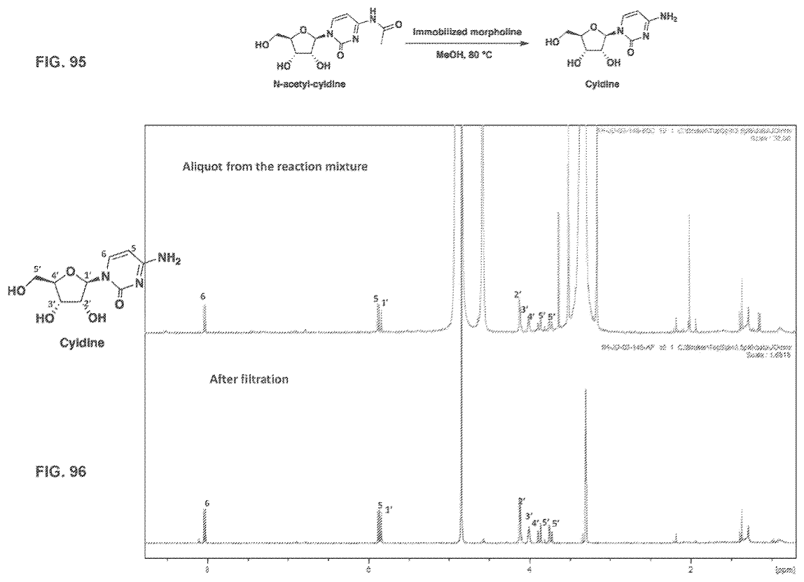

Deacylation of N-acetyl cytidine was tested using immobilized morpholine. The reaction scheme is shown in FIG. 95. A mixture of N-acetyl cytidine (100 mg, 0.35 mmoles) and immobilized morpholine (144 mg, 0.35 mmoles) was stirred in methanol (5 mL) at 80° C. for 24 h. Reaction progress was monitored by NMR. After deacetylation was complete, reaction mixture was filtered off, and the filtrate was evaporated to afford pure cytidine (85 mg, 100% yield). NMR spectra from the reaction mixture and after filtration are shown in FIG. 96.

Immobilized morpholine was dried and used for another batch. Nearly 85 mg of immobilized morpholine (0.2 mmoles) was obtained, which was recycled (used) to deacetylate another fresh batch.

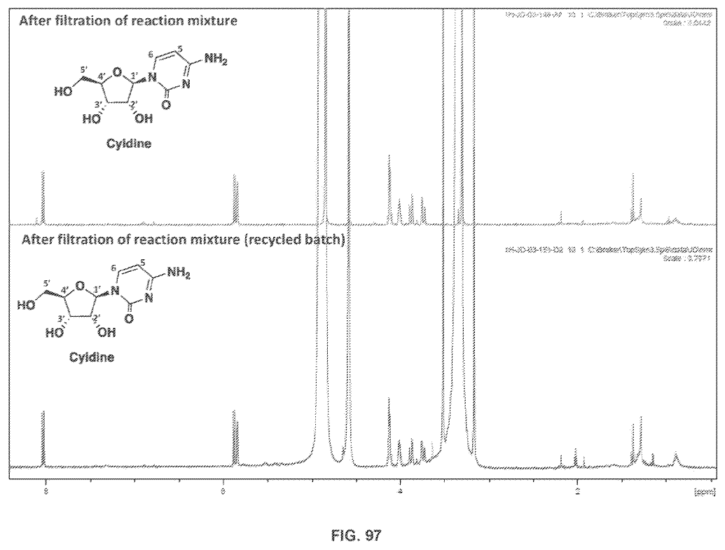

A mixture of N-acetyl cytidine (59 mg, 0.2 mmoles) and the recycled immobilized morpholine (85 mg, 0.2 mmoles) was stirred in methanol (5 mL) at 80° C. for 24 h in 5 mL of methanol. After deacetylation was complete, reaction mixture was filtered off and the filtrate was evaporated to afford pure cytidine. NMR spectra from the reaction mixture and after filtration are shown in FIG. 97.

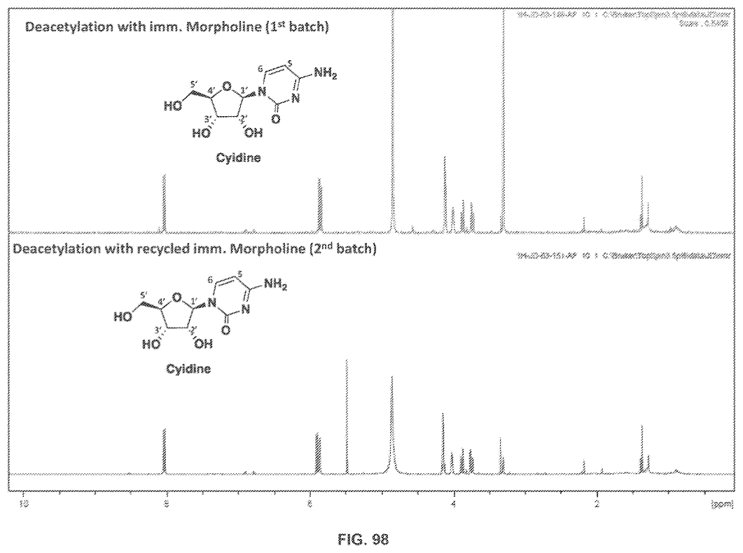

A comparison of the NMR spectra of cytidine produced using the first batch of immobilized morpholine vs. the second, recycled batch of immobilized morpholine is shown in FIG. 98. This Example demonstrates that the immobilized morpholine can be successfully recycled.

### F. Conclusion

It is to be understood that any given elements of the disclosed embodiments of the invention may be embodied in a single structure, a single step, a single substance, or the like. Similarly, a given element of the disclosed embodiment may be embodied in multiple structures, steps, substances, or the like.

The foregoing description and accompanying drawings illustrate and describe certain processes, machines, manufactures, and compositions of matter, some of which embody the invention(s). Such descriptions or illustrations are not intended to limit the scope of what can be claimed, and are provided as aids in understanding the claims, enabling the making and use of what is claimed, and teaching the best mode of use of the invention(s). If this description and accompanying drawings are interpreted to disclose only a certain embodiment or embodiments, it shall not be construed to limit what can be claimed to that embodiment or embodiments. Any examples or embodiments of the invention described herein are not intended to indicate that what is claimed must be coextensive with such examples or embodiments. Where it is stated that the invention(s) or embodiments thereof achieve one or more objectives, it is not intended to limit what can be claimed to versions capable of achieving all such objectives. Any statements in this description criticizing the prior art are not intended to limit what is claimed to exclude any aspects of the prior art.

Additionally, the disclosure shows and describes certain embodiments of the processes, machines, manufactures, compositions of matter, and other teachings disclosed. Still, it is to be understood that the teachings of the present disclosure are capable of use in various other combinations, modifications, and environments and are capable of changes or modifications within the scope of the teachings as expressed herein.

Any section headings herein are provided only for consistency with the suggestions of 37 C.F.R. § 1.77 or to provide organizational queues. These headings shall not limit or characterize the invention(s) set forth herein.

The following is claimed:

1. A method of deacylating an acylated carbohydrate, the method comprising: (a) forming a reaction mixture by mixing the acylated carbohydrate with a basic amino acid and methanol; and (b) allowing the methanol to react with an acyl group of the acylated carbohydrate.

2. A method of producing a deacylated carbohydrate from a corresponding acylated carbohydrate, the method comprising: (a) forming a reaction mixture by mixing the acylated carbohydrate with a basic amino acid and methanol; and (b) allowing the methanol to react with the acylated carbohydrate under conditions sufficient to form the deacylated carbohydrate.

3. A method of producing a composition comprising a basic amino acid and a deacylated carbohydrate, the method comprising: (a) forming a reaction mixture by mixing an acylated carbohydrate with a basic amino acid and methanol; (b) allowing the methanol to react with the acylated carbohydrate to form the deacylated carbohydrate and a methyl ester; and (c) removing unreacted methanol from the reaction mixture.

4. The method of claim 3, further comprising removing the methyl ester from the reaction mixture.

5. The method of any one of claims 1-4, wherein the method produces less than 25 ppm of methyl ester.

6. The method of any one of claims 1-5, wherein the method produces no detectable acetamide.

7. The method of any one of claims 1-6, further comprising removing the basic amino acid from the reaction mixture following step (b).

8. The method of any one of claims 1-7, wherein the acylated carbohydrate is a pentose poly or mono-ester.

9. The method of any one of claims 1-7, wherein the acylated carbohydrate is mono-acetylated, di-acetylated, or tri-acetylated.

10. The method of any one of claims 1-9, wherein the acylated carbohydrate comprises at least 5 carbons and is acetylated at one or more of its 2′, 3′, and 5′ carbons.

11. The method of any one of claims 1-10, wherein the acylated carbohydrate is a substituted ester.

12. The method of claim 11, wherein the acylated carbohydrate is a methyl substituted ester.

13. The method of claim 11, wherein the acylated carbohydrate is a C1-C10 ester.

14. The method of any one of claims 1-10, wherein the acylated carbohydrate is an ester of nicotinamide riboside.

15. The method of claim 14, wherein the acylated carbohydrate is an ester of reduced nicotinamide riboside.

16. The method of any one of claims 1-10, wherein the acylated carbohydrate is acetylated nicotinamide riboside.

17. The method of claim 16, wherein the acylated carbohydrate is triacetyl nicotinamide riboside.

18. The method of any one of claims 1-10, wherein the carbohydrate is a riboside.

19. The method of any one of claims 1-10, wherein the acylated carbohydrate is a pentose heteroarylamide.

20. The method of any one of claims 1-10, wherein the carbohydrate is a ribofuranoside compound.

21. The method of claim 20, wherein the carbohydrate is a D-ribofuranoside compound.

22. The method of any one of claims 1-10, wherein the carbohydrate is a nucleoside.

23. The method of any one of claims 1-10, wherein the carbohydrate is nicotinamide riboside.

24. The method of claim 23, wherein the carbohydrate is reduced nicotinamide riboside.

25. The method of any one of claims 1-10, wherein the acylated carbohydrate is a carbohydrate ester.

26. The method of any one of claims 1-7, wherein the acylated carbohydrate is a carbohydrate amide.

27. The method of any one of claims 1-7, wherein the acylated carbohydrate is a heteroaryl acetamide compound.

28. The method of any one of claims 1-7, wherein the acylated carbohydrate is a benzamide compound.

29. The method of any one of claims 1-10, wherein the acylated carbohydrate is a furanoside, and wherein the furanoside is acetylated at one or more of its 2′, 3′, and 5′ carbons.

30. The method of any one of claims 1-29, wherein a molar concentration of the methanol is at least the molar concentration of acyl groups in the acylated carbohydrate.

31. The method of any one of claims 1-30, wherein the method is performed at a temperature greater than or equal to 20° C.

32. The method of any one of claims 1-31, wherein the method is performed at about 20° C. to about 25° C.

33. The method of any one of claims 1-30, wherein the method is performed at a temperature greater than or equal to 40° C.

34. The method of any one of claims 1-33, wherein the basic amino acid is lysine.

35. The method of any one of claims 1-33, wherein the basic amino acid is arginine.

36. The method of any one of claims 1-35, wherein the basic amino acid is present at a molar concentration greater than or equal to a molar volume of the acylated carbohydrate.

37. The method of claim 36, wherein the basic amino acid is present at a molar concentration greater than or equal to three times the molar volume of the acylated carbohydrate.

38. The method of any one of claims 1-37, wherein the reaction is conducted under anhydrous and anoxic conditions.

39. The method of any one of claims 1-37, wherein the reaction mixture further comprises water.

40. The method of claim 39, wherein the reaction mixture comprises methanol and water at a ratio of about 1:30 to about 1:1.

41. The method of claim 39 or 40, wherein the method is performed at a temperature of about −30° C. to about 60° C.

42. The method of any one of claims 39-41, wherein the method is performed at a temperature of about −20° C. to about 50° C.

43. The method of any one of claims 39-42, wherein the method is performed at a temperature of about 0° C. to about 30° C.

44. The method of any one of claims 39-43, wherein the method is performed at a temperature of about 4° C. to about 25° C.

45. The method of claim 39 or 40, wherein the reaction mixture comprises methanol and water at a ratio of about 1:1, and wherein the method is performed at about −20° C.

46. The method of any one of claims 39-45, wherein the method produces at least 90% yield of the deacylated carbohydrate.

47. The method of any one of claims 39-46, wherein the method produces at least 95% yield of the deacylated carbohydrate.

48. The method of any one of claims 1-47, wherein the basic amino acid catalyzes methylation of the acyl group by methanol.

49. The method of any one of claims 1-48, comprising mixing the acylated carbohydrate with a basic amino acid and methanol by bead-beating.

50. The method of any one of claims 1-48, comprising mixing the acylated carbohydrate with a basic amino acid and methanol by stirring.

51. The method of any one of claims 1-48, comprising mixing the acylated carbohydrate with a basic amino acid and methanol by ball-milling.

52. The method of any one of claims 1-48, comprising mixing the acylated carbohydrate with a basic amino acid and methanol by co-extrusion.

53. A reaction mixture for deacylation of a carbohydrate, the reaction mixture comprising: a basic amino acid, the acylated carbohydrate, and methanol.

54. The reaction mixture of claim 53, further comprising water.

55. The reaction mixture of claim 54, comprising methanol and water at a ratio of about 1:10 to about 1:1.

56. A composition comprising a basic amino acid and a deacylated carbohydrate that is the product of a process comprising: (a) forming a reaction mixture by mixing an acylated carbohydrate with a basic amino acid and methanol; (b) allowing the methanol to react with the acylated carbohydrate to form the deacylated carbohydrate and a methyl ester; and (c) removing unreacted methanol from the reaction mixture.

57. The composition of claim 56, wherein the process further comprises removing the methyl ester from the reaction mixture.

58. The composition of claim 56 or 57, comprising less than 25 ppm of acetamide.

59. The composition of any one of claims 56-58, comprising no detectable acetamide.

60. The composition of any one of claims 56-59, comprising no detectable methanol.

61. The composition of any one of claims 56-60, comprising no detectable methyl ester.

62. The composition of any one of claims 56-61, wherein the reaction mixture further comprises water.

63. The composition of claim 62, wherein the reaction mixture comprises methanol and water at a ratio of about 1:10 to about 1:1.

64. The composition of any one of claims 56-63, wherein the reaction mixture comprises methanol and water at a ratio of about 1:1, and wherein the process is performed at about −20° C.

65. A composition formed by the method of any one of claims 1-52.
